(19) United States
(12) Patent Application Publication
(10) Pub. No.: US 2013/0267850 A1
(43) Pub. Date: Oct. 10, 2013

(54) SYSTEM AND METHOD FOR ULTRASONIC EXAMINATION OF THE BREAST

(71) Applicant: Michael Berman, Jerusalem (IL)

(72) Inventor: Michael Berman, Jerusalem (IL)

(21) Appl. No.: 13/992,091

(22) PCT Filed: Dec. 6, 2011
(86) PCT No.: PCT/IL11/50042

§ 371 Date: Jun. 6, 2013

Related U.S. Application Data
(60) Provisional application No. 61/420,098, filed on Dec. 6, 2010.

(51) Int. Cl. A61B 8/00; A61B 8/08

(52) U.S. Cl. CPC A61B 8/4461; A61B 8/0825; A61B 8/461
USPC 600/443

(57) ABSTRACT

The invention provides a system and method for limited view ultrasound imaging of a 2D section or a 3D volume of a body part. Ultrasound sensors configured are spatially or temporally arrayed in a limited view circular arc or over at least part of a concave surface such as a hemisphere. A processor calculates from detected ultrasound radiation a beam forming (BF) functional and calculates from the free amplitudes a point spread function (PSF). A filter g(k) is calculated from the Fourier transform H_{BF}(k) of the PSF that is used to generate an image of the 2D section or the 3D volume of the body part.

### PRIORITY INFORMATION

This National Stage application claims priority to U.S. Provisional Application No. 61/420,098 filed on Dec. 6, 2010.

### FIELD OF THE INVENTION

This invention relates to medical devices and more specifically to such devices for medical imaging by ultrasound.

### BACKGROUND OF THE INVENTION

The following prior art publications are considered to be relevant for an understanding of the prior art.
1. Report on “Mammography and beyond: developing technologies for the early detection of breast cancer”,
2. U.S.A. Institute of Medicine and the Governing Board of the National Research Council, National Academy Press, 2101 Constitution Avenue, N.W., Box 285, Washington, D.C. 20055.
3. Chandra M. Sehgal et al, Journal of Mammary Gland Biology and Neoplasia, Volume 11, Number 2, April, 2006.
4. W. E. Svensso, “Breast Ultrasound Update”, ULTRASOUND, February 2006, Volume 14, Number 1, 20-33.
5. J. F. Greenleaf et al on quantitative cross-sectional imaging of ultrasound parameters, Ultrasonics Symposium Proc., IEEE Cat. #77CH1264-1SU, pp. 989-995, 1977.
6. Littrup PJ et al Proceedings of the 26th International Acoustical Imaging Symposium, Windsor, Canada; Sep. 9-12, 2001.
7. An early patent issued in 1978 by Robert E. Frazer “Coupling apparatus for ultrasonic medical diagnostic system”, U.S. Pat. No. 4,130,112.
8. US patent Publication No. 20060241423 to Tor C Anderson et al.
9. US Patent publication 20060173307.
10. US patent publication 20070055159.
11. Thomas R. Nelson et al Proceedings of SPIE—Volume 6510, March 2007.
12. U.S. Pat. No. 4,509,368.
13. U.S. Pat. No. 7,025,725.
14. U.S. Pat. No. 7,264,592.
15. Karmanos Cancer Institute, Lawrence Livermore National Laboratory report [UCRL-JRNL-207220] of April 2004.
16. Lawrence Livermore National Laboratory report [UCRL-JRNL-207220] of April 2004.
17. International Patent Publication WO03/103500.
18. US patent publication number 20060241423.
19. U.S. Pat. No. 5,660,185.
20. U.S. Pat. No. 5,664,573.
21. A. Fenster et al, Ultrasound Med Biol. August; 27(8):1025-34, 2001.
22. J. F. Greenleaf et al Ultrasonics Symposium Proc., IEEE Cat. #77CH1264-1SU, pp. 989-995, 1977.
23. US patent publication No. 20060241423.
24. US Patent publication 20060173307.
25. US patent publication 20070055159.
26. U.S. Pat. No. 4,509,368.
27. U.S. Pat. No. 7,025,725.
28. U.S. Pat. No. 7,264,592.
29. U.S. Pat. No. 5,660,185.
30. U.S. Pat. No. 5,664,573.
31. Simonetti, F. & Huang, L. 2008, “From beamforming to diffraction tomography”, J. Appl. Phys. 103, 103 110.
32. US Patent Publication 2006/0009693.
33. Devaney, A. J. 1982, “A filtered backpropagation algorithm for diffraction tomography”, Ultrason. Imaging 4, 336-350.
34. Born, M. & Wolf, E. 1999 Principles of optics. Cambridge, UK: Cambridge University Press.
35. Kak, A. C. & Slaney, M. 1988 Principles of computerized tomographic imaging. New York, N.Y.: IEEE Press
36. Y. L. Luke, Integrals of Bessel Functions, McGraw-Hill, New York, 1962, p. 331 and 332.
37. http://en.wikipedia.org/wiki/Legendre_function.
38. http://en.wikipedia.org/wiki/Hypergeometric_function.
39. http://en.wikipedia.org/wiki/Gamma_function.
40. http://functions.wolfram.com/HypergeometricFunctions/Hypergeometric2F1/03/07/07/01/
41. http://en.wikipedia.org/wiki/Bessel_function
42. http://en.wikipedia.org/wiki/JacobiAnger_expansion
43. http://en.wikipedia.org/wiki/Angle_addition_formula#Angle_sum_and_difference_id entities
44. http://farside.ph.utexas.edu/teaching/jk1/lectures/node102.html (Spherical Harmonics)
45. W. Jarosz, N. Carr & H. W. Jensen, “Importance Sampling Spherical Harmonics”, Journal compilation, 2008, The Eurographics Association and Blackwell Publishing Ltd.
46. R Mehremt, J T Londergant and M H Macfarlanet, “Analytic expressions for integrals of products of spherical Bessel functions”, J. Phys. A: Math. Gen. 24(1991) 1435-1453.
47. Edmonds A R 1957 Angular Momentum in Quantum Mechanics (Princeton: Princeton University Academic Press
48. M. R. Aguilar, C. Elvira, A. Gallardo, B. Vazquez, and J. S. Roman, “Smart polymers and Their Applications as Biomaterials”, Topics in Tissue Engineering, Vol. 3, 2007. Eds. N Ashammakhi, R Reis & E Chiellini© 2007.
49. S. F. Khattak, S. R. Bhatia, S. C. Roberts, “Pluronic F127 as a Cell Encapsulation Material: Utilization of Membrane-Stabilizing Agents”, Tissue Engineering Volume 11, Number 5/6, 2005

Breast cancer is one of the leading causes of death from cancer. Early detection is widely believed to reduce breast cancer mortality by allowing intervention at an earlier stage of cancer progression. Screening [X-ray] mammography has secured a place as the gold standard routine health maintenance procedure for women—this is a mature technology that provides high-quality images in the majority of patients.

However, conventional mammography does not detect all breast cancers, including some that are palpable, and as many as three-quarters of all breast lesions biopsied because of a suspicious finding on a mammogram turn out to be benign.

Mammograms are particularly difficult to interpret for women with dense breast tissue, who are at increased risk of breast cancer. The dense tissue interferes with the identification of abnormalities associated with tumors. Accordingly, other imaging technologies, particularly non-ionizing modalities such as magnetic resonance imaging and ultrasound, are being tested for application to breast cancer. These methods may provide additional diagnostic specificity over X-ray mammography alone. These include ultrasound examinations, and needle or surgical biopsies. Improvements in these techniques are desirable for more accurate and less invasive treatment.

Ultrasound examinations are routinely done following a suspicious finding, for example in order to discriminate a cyst from a solid mass. Furthermore, for women with dense breast tissue, ultrasound is used even for screening. However, the varied, heterogeneous, complex structure of the breast makes good breast ultrasound imaging more difficult than in many other regions of the body. Conventional ultrasound has a limited field of view, is not reproducible and produces results that are a trade-off between penetration and image resolution. It is generally perceived that ultrasound is incapable of reliably detecting micro-calcifications, these being early indications of breast cancer. Conventional two dimensional [2D] ultrasound procedures dynamically distort the breast during the examination, thereby making it difficult to determine the exact location of tumors or other masses. The 2D nature of conventional ultrasound and the distortion of the breast during the examination, make it difficult to guide biopsy or ablation procedures.

Several approaches to introducing 3D breast ultrasound procedures have been attempted in the prior art. These include conventional 3D scanning as well as Ultrasound Computed Tomography.

The conventional 3D scanning approach is driven by the need for real-time data acquisition and display. Therefore, some of the complex physics associated with propagation of sound waves is traded off. One of the tradeoffs corresponds to the usage of straight-ray theory, a basic approximation of the true physics of acoustic wave propagation, which is only valid for purely homogenous media. A second important tradeoff is the assumption of a 2D geometry in which only the directly backscattered reflections are collected. In reality, the emitted pulses interact so strongly with the tissue that a “scattered field” of acoustic energy is produced and acoustic waves are distributed in all directions. This means that in conventional 3D ultrasound, only a small fraction of the scattered waves arrive at the detectors. Consequently, the strength of the detected signal is weak and it is necessary to “beam form” (focus energy in a specific direction) in order to amplify the return signal. 3D and also four-dimensional [4D] standard transducers are also used for breast imaging. The main motivation behind their usage is the need for real-time data acquisition and display. As with 2D conventional ultrasound, the distortion of the breast during the examination with such transducers disturbs the determination of the exact location of tumors or other masses and makes it difficult to guide biopsy or ablation procedures. In addition, as in conventional 2D ultrasound, these transducers detect back scattered sound waves only, thus lacking the advantages introduced by tomography methods discussed below.

A tomography approach may “undo” these trade-offs, leading to a marked increase in the signal-to-noise ratio, while reducing artifacts and yielding higher quality images for greater clinical sensitivity. Furthermore, the signals that propagate through the breast, and which are never reflected back, contain additional information. These transmitted signals can be used to calculate acoustic parameters, not contained in the reflection data, such as sound speed and attenuation, possibly leading to greater clinical specificity. Prior art contributions to this conclusion include the pioneering paper by J. F. Greenleaf et al. [22].

One of a variety of obstacles to developing a device capable of facilitating scanning of the whole breast volume in a short time is the difficulty of maintaining good contact between the ultrasound transducer and the skin and the image quality that results from a controlled but inflexible scanning mechanism.

A full-field breast ultrasound (FFBU) scanning apparatus and related methods were suggested in the prior art. For example, US patent Publication: 20060241423 of Anderson et al. discloses such a device. However, this device required the compression of the breast.

US Patent Publication 20060173307 of Amara Arie et al describes a circular breast ultrasound scanner (CBUS). The CBUS system automatically images the complete breast. A mechanical device for moving a standard 2D ultrasound transducer in a circular or helical motion is used. The breast is placed in a hollow [vacuum] housing.

US Patent Publication 20070055159 to Wang Shih-Ping et al, describes an apparatus and related methods for facilitating volumetric ultrasonic scanning of a breast. A cone-shaped radial scanning template is rotated, thereby moving an ultrasound transducer to scan the breast. A flexible membrane is mounted on a mechanical assembly to form a slot-like opening through which an ultrasound transducer directly contacts the skin surface. The breast in this procedure has to be compressed.

The publication of Thomas R. Nelson et al [11] discloses a volume breast ultrasound scanner that images a pendant breast. Their performance assessment characterized a variety of parameters including: spatial resolution, uniformity, and distortion using high and low contrast test objects in both horizontal and vertical scanning modes. Test object images depicted hyper- and hypo-echoic masses and demonstrated good resolution, soft tissue contrast and reduced speckle compared to conventional ultrasound images.

U.S. Pat. No. 4,509,368 to Whiting et al. [26], discloses a method and apparatus for ultrasound tomography for use in clinical diagnostics. The apparatus comprises paired couples of transmission transducers and reflection transducers, independently operable within a container.

U.S. Pat. No. 7,025,725 to Donald P. Dione et al, [27] discloses an imager having a plurality of cylindrical rings, generating a signal in a cone beam form, U.S. Pat. No. 7,264,592, to Shehada, Ramez E. N. discloses a breast tomography scanner configured to hold fluid within stationary and movable chambers into which a breast is immersed.

A Lawrence Livermore National Laboratory report [UCRL-JRNL-207220] [16] describes ultrasound tomography using ring geometry for breast imaging. A breast phantom was immersed in a fluid bath. The spatial resolution, deduced from images of reflectivity, was 0.4 mm. The demonstrated 10 cm depth-of-field was superior to that of conventional ultrasound and the image contrast was improved through the reduction of speckle noise and overall lowering of the noise floor. Images of acoustic properties such as sound speed suggested that it is possible to measure variations in the sound speed of 5 m/s. An apparent correlation with X-ray attenuation suggested that the sound speed can be used to discriminate between various types of soft tissue.

With devices that employ liquid-filled coupling chambers into which the breast is immersed, the transducer is inevitably sited away from the tissue and hence the focus of the ultrasound beam is poor.

International Patent Publication WO03/103500 [17] discloses a device having a mounting structure capable of holding an ultrasound transducer and a tissue molding element for receiving and surrounding the breast tissue.

Another system that avoids use of a liquid bath is described by in US Patent Publication 20060241423 of Anderson et al. One side of the breast is compressed by a membrane or film sheet and the other side of the breast is compressed by a rigid plate and an inflatable air bladder. The transducer surface is held against a second surface of the film. An irrigation system is used to maintain a continuous supply of coupling agent at an interface between the transducer surface and the film sheet as the transducer is translated

Examples of proposed devices for ultrasound-assisted biopsy procedures are disclosed in: U.S. Pat. Nos. 5,660,185 and 5,664,573. A further development and evaluation of a three-dimensional ultrasound-guided breast biopsy apparatus is described in Fenster et al [21].

Diffraction tomography (DT) using ultrasound produces a stack of 2D tomography images, similar to those obtained by X-ray or magnetic resonance (MR) tomography. Compared with X-ray or MR DT images, ultrasound CT images do not use potentially harmful ionizing radiation.

A planar section of a body part being imaged is described by an object function O(r,ω), which for acoustic waves and a lossless object, is given by

$$O ( r , ω ) = {k}_{0}^{2} [ {( \frac{{c}_{0}}{c ( r , ω )} )}^{2} - 1 ] \qquad ( 1 )$$

where r is the direction of the incident plane wave, c₀ is the speed of sound in the homogeneous background in which the object is immersed, c(r,ω) is the local sound speed inside the object, and k₀ is the background wave number, 2π/λ where λ is the wave length. The goal of DT is to determine the objection function O(r,ω) from a series of diffraction experiments and to generate an image from the object function.

In ultrasound DT of the breast an array of ultrasound transducers positioned around a ring is used. A breast to be examined is inserted into the ring, and ultrasound images are obtained at each of a sequence of positions of the ring as the ring is moved relative to the breast along an axis essentially perpendicular to the body surface. Each layer of the breast is thus probed using an array of ultrasound transducers arranged along a 360° arc, so that each layer of the breast is probed from essentially all directions. A system for imaging a breast that scans the breast with a circular array of ultrasound transducers is disclosed, for example, in US Patent Publication 2006/0009693[32].

DT over an entire circle generates a low-pass-filtered image given by Õ(k)Π(|k|) where Õ(k)=∫_{-∞}^{∞}d²r O(r)e^{-ik·r} is the two-dimensional Fourier transform of the object function O(r) and

$$Π ( \vert k \vert ) = \{ \begin{matrix}1 & \vert k \vert < 2 {k}_{0} \\ 0 & \vert k \vert > 2 {k}_{0} ,\end{matrix}$$

where k₀ is the incident wave number.

DT over an entire circle has the advantage that the breast is probed from all directions. However, due to the anatomy of the breast, the breast can be probed from all directions only for planar sections essentially perpendicular to the axis of the breast. Moreover, because the ring of transducers has a fixed diameter, the distance between the ring and the breast is not uniform as the breast is scanned due to the tapering of the breast towards the nipple.

Beam forming (BF) methods are an integral part of ultrasound imaging with well known engineering and algorithmic technologies. Consider a body part probed from directions along a limited view circular arc having a central angle 0<ξ<2π. In BF, an object function O(r,ω) is generated by focusing an incident beam from each of a plurality of directions along the circular arc, and for each of these directions of incident acoustic wave, the amplitudes of the scattered rays are determined. The output of this scattering measurement is a set of amplitudes ƒ(φᵣ,φₜ), where φₜ is the angle of the incident wave with respect to a fixed radius of the circular arc, and φᵣ is the direction of the scattered wave with respect to the fixed radius. The measured ƒ(φᵣ, φₜ) are phase shifted and integrated over the aperture of the array, so that only the contributions to the scattered field from the focal point are added coherently. As shown by Simonetti and Huang 2008 [31], for a continuous set of transducers, this two-step process is obtained by means of the BF functional

$${𝒥}_{BF} = {\int }_{0}^{ξ} d {φ}_{r} {\int }_{0}^{ξ} d {φ}_{t} \times exp [ - i {k}_{0} \hat{u} ( {φ}_{r} ) \cdot z ] f ( {φ}_{r} , {φ}_{t} ) exp [ i {k}_{0} \hat{u} ( {φ}_{t} ) \cdot z ] \qquad ( 2 )$$

where û is the unit vector associated with the angle φ.

The point spread function associated with the BF functional (2) can be obtained from the free-scattering amplitude defined by:

ƒ_{free}(φᵣ,φₜ)=Nexp{−ik₀[û(φₜ)−û(φᵣ)]·r} (3)

where

$$N = \frac{exp ( \frac{i π}{4} )}{\sqrt{8 π {k}_{0}}} .$$

The beam forming Point Spread function (PSF), also called the Spatial Impulse Response (SIR) is given by

$${h}_{BF} ( z - r ) = N {\int }_{0}^{ξ} d {φ}_{r} {\int }_{0}^{ξ} d {φ}_{t} \times exp \{ - i {k}_{0} \hat{u} ( {φ}_{r} ) \cdot ( z - r ) \} exp ( i {k}_{0} \hat{u} ( {φ}_{t} ) \cdot ( z - r ) \} \qquad ( 4 )$$

An object O(r) function can be obtained from the relation: I_{BF}(k)=Õ(k) H_{BF}(k), where I_{BF}(k), Õ(k) and H_{BF}(k) are the two-dimensional Fourier transform of the BF functional ℑ_{BF}, O(r), and h_{BF}(z−r), respectively. The image I_{BF}(k) generated from an object function in a beamforming proceedure in which the object is probed from an entire circular arc will in general appear distorted in comparison to an image obtained from a Diffraction Tomography experiment.

While the “full world” (entire circular arc in a two dimensional world) relationship between beamforming and tomography has been investigated, the extension to a limited-view world (limited-view circular arc in two dimensions and limited-view sphere, such as a hemi-sphere in the three-dimensional world) is not known in the prior art.

### SUMMARY OF THE INVENTION

In its first aspect, the present invention provides a system for imaging a body part using ultrasound radiation.

In one embodiment of the invention, a planar section of the body part is probed using an array of ultrasound transducers that are spatially or temporally arranged on a limited view circular arc having a central angle 0<ξ<2π. In the case of imaging a breast, the use of an arc of transducers allows images of planar sections of the breast to be obtained that are not necessarily perpendicular to the axis of the breast. The beam can be applied onto the breast in a plurality of orientations that are not necessarily perpendicular to the axis of the breast. Thus, planar sections of the breast may be probed sequentially by moving a single array of transducers over the breast, in a variety of directions.

In this embodiment of the invention, the system includes a processor that is configured to generate an image from a planar section of a body part from the amplitudes ƒ(φᵣ, φₜ). The inventor has derived an explicit form of the filter g(k) in the relationship

I_{BF}(k)=Õ(k)H_{BF}(k)=g(k)Õ(k)Π(|k|) (5)

where I_{BF}(k) is the two-dimensional Fourier transform of the limited-view BF functional ℑ_{BF} in equation (2), H_{BF}(k) is the two-dimensional Fourier transform of h_{BF}(z−r) in equation (4). The derivation of (5) is presented in Annex A. As the term Õ(k)Π(|k|) represents the known result for the full-view diffraction tomography, the filter g(k) constitutes a mapping to obtain the diffraction tomography result. This filter g(k) can be obtained from an explicit form of the limited view H_{BF}(k) (as derived in Annex A):

$${H}_{BF} ( k ) = N {\sum }_{{n}_{2} = - ∞}^{∞} {\sum }_{{n}_{1} = - ∞}^{∞} \frac{2 π {i}^{2 {n}_{1} - 2 {n}_{2}}}{{n}_{1} {n}_{2}} ( {e}^{i ( {n}_{1} - {n}_{2} ) ξ} - {e}^{- i {n}_{2} ξ} - {e}^{i {n}_{1} ξ} + 1 ) {e}^{i ( {n}_{2} - {n}_{1} ) φ} {I}_{{n}_{1} , {n}_{2} , {n}_{2} - {n}_{1}}$$

where Iₙ_{₁}_{,n}_{₂}_{,n}_{₂}₋ₙ_{₁} are integrals of products of 3 Bessel Functions of orders n₁, n₂, n₂−n₁. These integrals are shown in Annex A to include the low pass filter Π(|k|) linearly, i.e H_{BF}(k)=g(k)Π(|k|), thereby defining the explicit form of the filter g(k).

In accordance with this embodiment of the invention, the processor first calculates I_{BF}(k) as explained above. The I_{BF}(k) is then multiplied by the inverse of the filter (k), to yield Õ(k)Π(|k|), which is the generated tomographic image of the planar section of the body part. Consequently, a tomographic image is generated using beamforming technology.

In another embodiment of the invention, a three-dimensional section of a body part is probed using an array of ultrasound transducers that are spatially or temporally arranged on a curved surface such as a hemi-sphere. In this embodiment, the scattering amplitudes are given by a function ƒ(θᵣ, θₜ, φᵣ, φₜ), where (θₜ, φₜ) are the spherical coordinates of the transmitted beam and (θᵣ, φᵣ) are the spherical coordinates of the reflected beam, θᵣ, θₜε[0,π] and φᵣ, φₜε[0,π], (for a shpere φᵣ, φₜε[0, 2π]). Standard BF produces the image of an object at a point z, of the image space by focusing an incident beam at r=z in the object space. As in the two dimensional (planar) embodiment, the resulting scattered field is subsequently phase shifted and integrated over the aperture of the array, so that only the contributions to the scattered field from the focal point are added coherently. This two-step process is obtained by means of the 3D BF functional:

$${𝒥}_{BF} = {\int }_{D} exp [ i {k}_{0} \hat{u} ( {θ}_{r} , {φ}_{r} ) \cdot z ] f ( {θ}_{r} , {θ}_{t} , {φ}_{r} , {φ}_{t} ) exp [ i {k}_{0} \hat{u} ( {θ}_{t} , {φ}_{t} ) \cdot z ]$$

where D is a limited view domain on the sphere. For the special case of a semi-sphere it reads:

ℑ_{BF}=∫₀^{π}dφᵣ∫₀^{π}dθᵣ sin θᵣ∫₀^{π}dφₜ∫₀^{π}dθₜ sin θₜ×exp[ik₀û(θᵣ,φᵣ)·z]ƒ(θᵣ,θₜ,φᵣ,φₜ)exp[ik₀û(θₜ,φₜ)·z] (6)

in the above equations, û is the unit vector associated with the angles θ and φ. As in the two-dimensional embodiment, the second exponential in equation (6) represents focusing in transmission, whereas the first corresponds to the focusing of the received scattered field. The point spread function (PSF) associated with the functional (6) can be obtained by considering the image of a point scatterer at position r. In this case, the three-dimensional free-scattering amplitude is

ƒ_{free}(θᵣ,θₜ,φᵣ,φₜ)=exp{−ik₀[û(θₜ,φₜ)+û(θᵣ,φᵣ)]·r} (7)

and the three-dimensional PSF reads

h_{BF}=∫₀^{π}dφᵣ∫₀^{π}dθᵣ sin θᵣ∫₀^{π}dφₜ∫₀^{π}dθₜ sin θₜ×exp{ik₀û(θᵣ,φᵣ)·[z−r]}exp{ik₀û(θₜ,φₜ)·[z−r]} (8)

The inventor developed an analytic expression for the three-dimensional Fourier transform, H_{BF}, of h_{BF}(z−r) in the form:

$${H}_{BF} = g ( k ) Π ( \vert k \vert ) , where Π ( \vert k \vert ) = \{ \begin{matrix}1 & \vert k \vert < 2 {k}_{0} \\ 0 & \vert k \vert > 2 {k}_{0}\end{matrix} \qquad ( 9 )$$

The derivation of (9) is presented in Annex B. The DT problem consists of reconstructing the function O(r) from a set of scattering experiments. The object function in the spatial frequency domain, K-space, which is obtained by performing the three-dimensional Fourier transform of O(r) may be represented by:

$$\tilde{O} ( k ) = {\int }_{- ∞}^{∞} {d}^{3} r O ( r ) {e}^{- e - i k \cdot r} \qquad ( 10 )$$

The beamforming image:

ℑ_{BF}=∫_{-∞}^{∞}dr₁∫_{-∞}^{∞}dr₂∫_{-∞}^{∞}dr₃O(r)h(|z−r|) (11)

in the spatial frequency domain reads (using equation 8)

I_{BF}(k)=Õ(k)H_{BF}(k)=g(k)Õ(k)Π(|k|) (12)

The derivation of (12) is presented in Annex B.

While DT over an entire sphere leads to the low-pass-filtered image, Õ(k)Π(|k|), the BF algorithm introduces a distortion that is described by the additional filter g(k) in equation (12). As a result, the DT image can be obtained from the BF image by applying the filter

$$\frac{1}{g ( k )}$$

to the BF image.

In another of its aspects, the present invention provides an ultrasound apparatus and method for guiding procedures, such as biopsy or ablation in a body part. Real-time three-dimensional images (“4-D ultrasound imaging”) for procedure guidance are superimposed on top of high-resolution tomographic images by spatial registration. This is enabled by mechanically coupling a two-dimensional array transducer for producing three dimensional real-time imaging, to an array of ultrasound transducers arranged on a circular arc of the two dimensional embodiment of the first aspect of the invention or to an array of ultrasound transducers arranged on a hemi-sphere of the three-dimensional embodiment of the first aspect of the invention for producing tomographic imaging.

Thus, in one of its aspects, the invention provides a system for limited view ultrasound imaging of a 2D section or a 3D volume of a body part comprising:

(a) one or more ultrasound sensors, the ultrasound sensors being configured to be spatially or temporally arrayed in an array selected from:
(i) a limited view circular arc having a central angle ξ, ξ satisfying 0<ξ<2π, the ultrasound sensors generating a plurality of amplitudes ƒ(φᵣ, φₜ), where ƒ(φᵣ, φₜ) is an amplitude of ultrasound radiation in a direction forming an angle φᵣ, with a fixed radius of the limited view circular arc when the body part is probed with incident radiation from a direction forming an angle φₜ with the fixed radius; wherein 0<φᵣ,φₜ<ξ;
(ii) a concave surface, the ultrasound sensors generating a plurality of amplitudes ƒ(θᵣ, θₜ, φᵣ, φₜ), where ƒ(θᵣ, θₜ, φᵣ, φₜ) is an amplitude of ultrasound radiation when the body part is probed from a transmit direction determined by angles θₜ, φₜ and a receive direction determined by angles θᵣ, φᵣ wherein θᵣ, θₜε[0, π] and φᵣ, φₜε[0,π];
(b) a processor configured to:
calculate from the ƒ(φᵣ, φₜ) or the ƒ(θᵣ, θₜ, φᵣ, φₜ) a beam forming (BF) functional;
calculate free amplitudes ƒ_{free}(φᵣ, φₜ) or ƒ_{free}(θᵣ, θₜ, φᵣ, φₜ);
calculate from the free amplitudes ƒ_{free}(φᵣ, φₜ) or ƒ_{free}(θᵣ, θₜ, φᵣ, φₜ) a point spread function (PSF);
calculate a filter g(k) from the Fourier transform H_{BF}(k) of the PSF;
calculate a Fourier transform I_{BF}(k), of the BF functional;
divide I_{BF}(k), by the filter g(k) to yield Õ(k)Π(|k|); and
generate an image of 2D section or the 3D volume of the body part using the Õ(k)Π(|k|).

The system of the invention may further comprise a scanning device including a dome shaped structure wherein the ultrasound sensors are configured to be spatially or temporally arrayed over at least a portion of the dome structure. The dome shaped structure may be configured to be placed over a breast of a female individual. The dome shaped structure may include a layer formed from an acoustically transparent material.

The system of the invention may comprise one or more C-Arm-tomography-sensors and one or more 2D-array-sensors. The sensors may be connected to a step-motor-assembly configured to drive the ultrasound sensors over the scanning device. The step-motor-assembly may include any one or more of a motor, an encoder, a processor, an indexer and a driver. The C-arm-tomography-transducer may be moved, for example, along a circular-track.

The system of the invention may further comprise a display device and the processor may be configured to display the image on the display device. The processor may be further configured to superimpose on a displayed image one or more B-Mode compounded images or tomography images.

The system of the invention may further comprise a garment configured to be worn by an individual over the body part, the garment comprising a layer formed from a thermo-responsive-acoustic-transparent-polymer, being in a first viscous state at a first temperature below 37° C. and in a second viscous state at a second temperature above 37° C., the second viscous state having a viscosity above a viscosity of the first viscous state. The garment, may be, for example, a bra.

The system of the invention may further comprise a chair wherein the scanning device is positioned in the chair with the dome in an inverted orientation.

The thermo-responsive-acoustic-transparent-polymer layer may be harder at an outer surface as compared to an inner surface that is in contact with the body part.

The dome may comprise one or more holes configured to receive a biopsy needle.

The invention also provides a garment for use in the system of the invention, the garment being configured to be worn by an individual over the body part, the garment comprising a layer formed from a thermo-responsive-acoustic-transparent-polymer.

The invention also provides a chair for use in the system of the invention, wherein the scanning device is positioned in the chair with the dome in an adjustable orientation including an inverted orientation.

The system of the invention may further comprise a 2D array of ultrasound sensors mechanically coupled to a C-arm tomographic arc or to the concave surface and the generated image may be a real-time 3D image.

In another of its aspects, the invention provides a method for limited view ultrasound imaging of a 2D section or a 3D volume of a body part comprising:

(a) providing one or more ultrasound sensors, the ultrasound sensors being configured to be spatially or temporally arrayed in an array selected from:
(i) a limited view circular arc having a central angle ξ, ξ satisfying 0<ξ<2π, the ultrasound sensors generating a plurality of amplitudes ƒ(φᵣ, φₜ), where ƒ(φᵣ, φₜ) is an amplitude of ultrasound radiation in a direction forming an angle φᵣ with a fixed radius of the limited view circular arc when the planar section is probed with incident radiation from a direction forming an angle φₜ with the fixed radius; wherein 0<φᵣ,φₜ<ξ.
(ii) a concave surface, the ultrasound sensors generating a plurality of amplitudes ƒ(θᵣ, θₜ, φᵣ, φₜ), where ƒ(θᵣ, θₜ, φᵣ, φₜ) is an amplitude of ultrasound radiation when the body part is probed from a transmit direction determined by angles θₜ, φₜ and a receive direction determined by angles θᵣ φᵣ the angles satisfying θᵣ, θₜε[0, π] and φᵣ, φₜε[0, π],
(b) calculating from the ƒ(φᵣ, φₜ) or the ƒ(θᵣ, θₜ, φᵣ, φₜ) a beam forming (BF) functional;
(c) calculating free amplitudes ƒ_{free}(φᵣ, φₜ) or the ƒ_{free}(θᵣ, θₜ, φᵣ, φₜ)
(d) calculating from the free amplitudes ƒ_{free}(φᵣ, φₜ) or the ƒ_{free}(θᵣ, θₜ, φᵣ, φₜ) a point spread function (PSF);
(e) calculating a filter g(k) from the Fourier transform H_{BF}(k) of the PSF;
(f) calculating a Fourier transform I_{BF}(k), of the BF functional;
(g) dividing I_{BF}(k), by the filter g(k) to yield Õ(k)Π(|k|); and
(h) generating an image of 2D section or the 3D volume of the body part using the Õ(k)Π(|k|).

The body part may be, for example, a breast.

The method of the invention may further comprise spatially or temporally arraying the ultrasound sensors over at least a portion of the dome structure. The dome shaped structure may include a layer formed from an acoustically transparent material.

The method of the invention may further comprise displaying the image on a display device. The method may further comprise superimposing on a displayed image one or more B-Mode compounded images or tomography images.

The method of the invention may further comprise placing a garment on an individual over the body part, the garment comprising a layer formed from a thermo-responsive-acoustic-transparent-polymer, the thermo-responsive-acoustic-transparent-polymer being in a first viscous state at a first temperature below 37° C. and in a second viscous state at a second temperature above 37° C., the second viscous state having a viscosity above a viscosity of the first viscous state.

In the method of the invention, the scanning device may positioned in a chair with the dome in an adjustable orientation including an inverted orientation, and the method further comprising placing the body part in the dome. A thereto-responsive-acoustic-transparent-polymer that is in a first viscous state at a first temperature below 37° C. and in a second viscous state at a second temperature above 37° C., the second viscous state having a viscosity above a viscosity of the first viscous state may be introduced into the dome. The thermo-responsive-acoustic-transparent-polymer layer may be harder at an outer surface as compared to an inner surface that is in contact with the body part.

The method of the invention may further comprise inserting a biopsy needle in a hole in the dome structure and obtaining a biopsy.

The method of the invention may utilize a 2D array of ultrasound sensors that is mechanically coupled to a C-arm tomographic arc or to the concave surface and the method may further comprise generating a real-time 3D image. The real-time 3D image may be used in guiding a surgical or procedure tool through the body part.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 shows a system for limited view imaging of a body part in accordance with one embodiment of the invention;

FIG. 2 shows a scanning-device placed on breast for use in the system of FIG. 1;

FIG. 3 shows internal parts of the scanning device of FIG. 2;

FIG. 4 shows a cover of the scanning device of FIG. 2 from a front view (FIG. 4a), a left side view (FIG. 4b), a tilted view (FIG. 4c) and a right side view (FIG. 4d);

FIG. 5 shows the cup of a bra including a layer of a thermo-responsive acoustic transparent polymeric;

FIG. 6 shows a C-Arm tomography transducer of the scanning device of FIG. 2;

FIG. 7 shows a 2D-Array transducer of the scanning device of FIG. 2;

FIG. 8 shows a C-arm transducer (FIG. 8a) and a 2D-array transducer (FIG. 8b) of the scanning device of FIG. 2;

FIG. 9 shows schematically the step motor of the scanning device of FIG. 2;

FIG. 10 shows the C-arm and 2D-array transducers of the scanning device of FIG. 2 from a top view (FIG. 10a), a diametric view (FIG. 10b), a front view (FIG. 10c) and a right side view (FIG. 10d).

FIG. 11 shows a chair for use in the system of the invention;

FIG. 12 shows the scanning device of FIG. 2; and

FIG. 13 shows the step motor of the scanning device of FIG. 12.

### DETAILED DESCRIPTION OF EMBODIMENTS

For the sake of clarity, and ease of description, the present invention will be described in relation to breast imaging, it being evident that the system and method of the invention can be modified to image any desired body part.

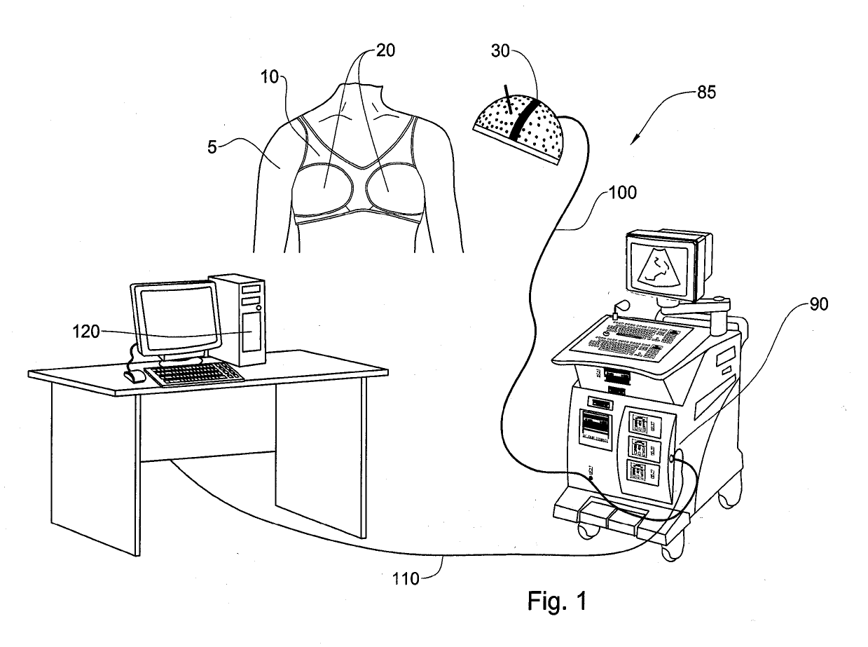

FIG. 1 shows a system 85 for ultrasound imaging of a breast in accordance with one embodiment of the invention. The system 85 comprises a dome shaped scanning device 30, described in detail below, configured to receive in its interior a breast of an individual 5. The scanning-device 30 is anchored to an ultrasound system 90 over a cable-assembly 100. A control-cable 110 connects the ultrasound system 90 to a workstation 120. The work station 120 may include a CRT screen 123 for displaying images. A user input device, such as a keypad 124 allows a user to input various parameters relating to the examination, such as personal details of the individual being examined, or the parameters of the ultrasound radiation (frequency, intensity, etc.).

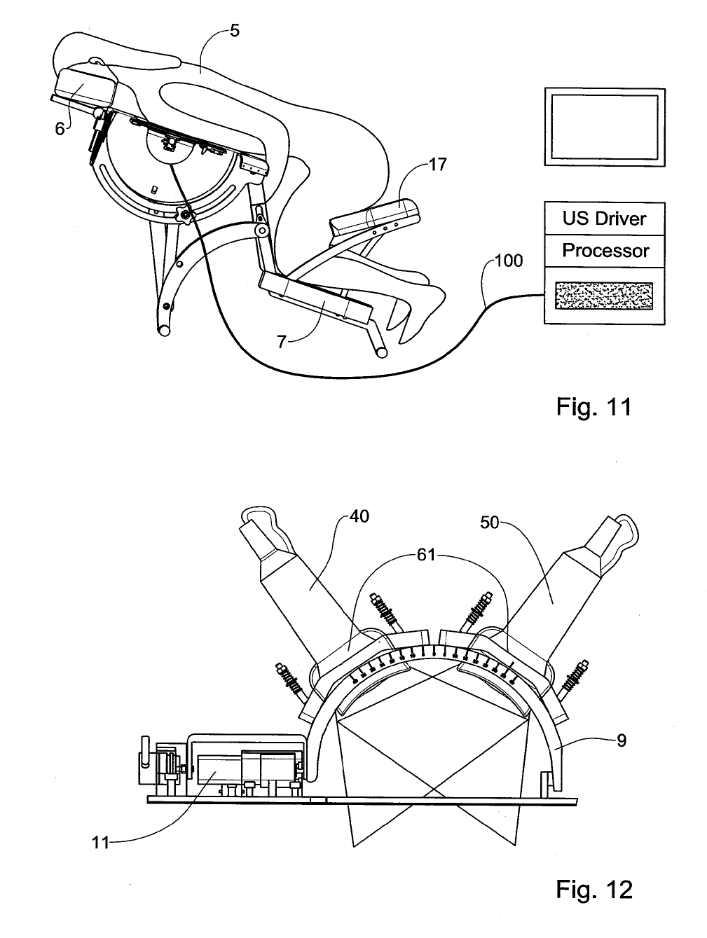

In one embodiment of the invention, shown in FIG. 1, the system includes a bra 10 configured to be worn by an individual 5 and the scanning device 30 is configured to be placed on a breast over a cup 20 of the bra. In another embodiment of the invention, shown in FIG. 11, the scanning device 30 is incorporated into a chair 7 having a seat 17 upon which the individual 5 is seated. The scanning device 30 is positioned in the chair 7 with its opening on top. The individual 5 sits on the seat 17 and inserts a breast to be imaged into the scanning device 30. The chair 7 can be aligned in a variety of positions to accommodate individuals of different sizes.

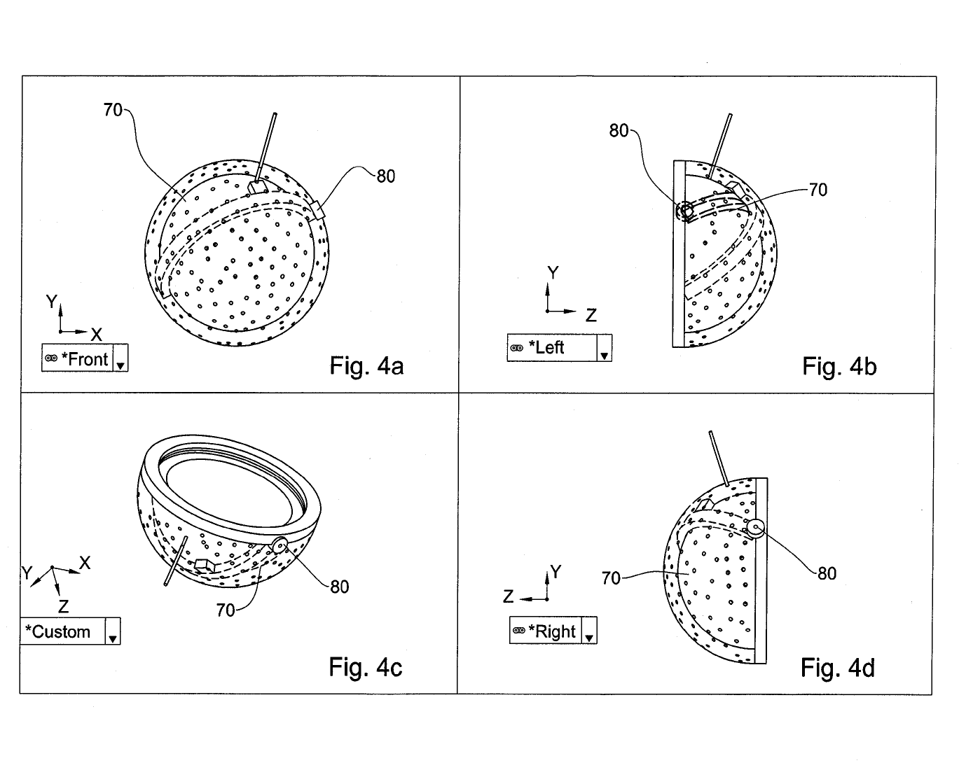

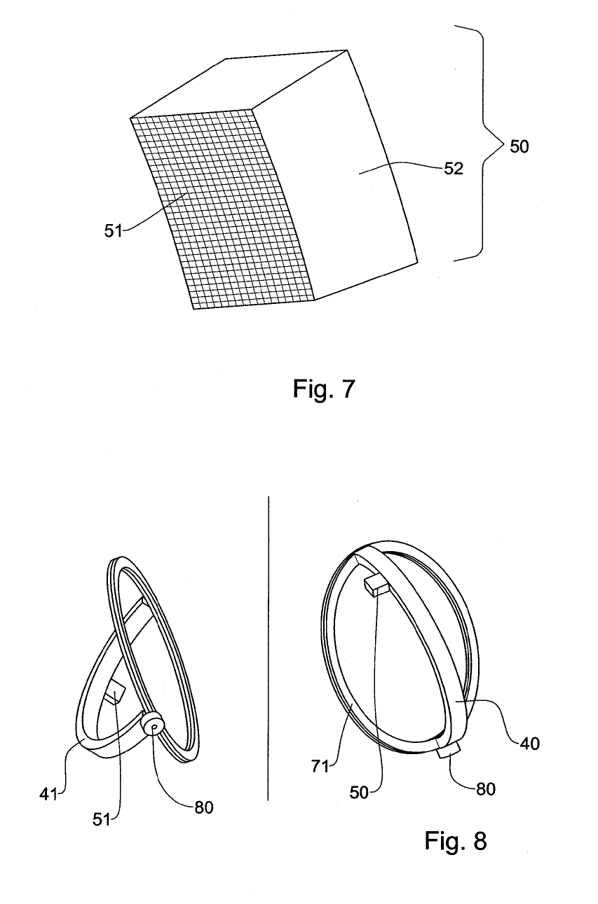

FIGS. 3 to 10, 12 and 13 show the scanning device 30 in greater detail. Referring first to FIG. 3, the scanning device includes a dome structure 21 made from an acoustic-transparent-polymer such as Aqualene™. A C-Arm-tomography-transducer 40 and a 2D-Array-transducer 50 are positioned on top of the dome 20. The transducers 40 and 50 are connected to a step-motor-assembly 80, which is connected to a scanning-device cover 70 having needle-holes 71. The cover 70 is shown in greater detail in FIG. 4, which shows a front view (FIG. 4a), a left side view (FIG. 4b), a tilted view (FIG. 4c), and a right side view (FIG. 4d) of the cover 70 of-scanning-device connected to the step-motor-assembly 80. In use, the dome 20 is positioned between the breast and the transducers 40 and 50 which are in contact with the outer-surface 22. Closer views of the transducers 40 and 50 are shown in FIGS. 6 and 7. In FIG. 6 is shown a concave acoustic-stack 41 of the C-Arm-tomography-transducer 40. Also shown in FIG. 6 is a sliding-track 42 of the C-Arm-tomography-transducer 40. FIG. 7 shows the acoustic-stack 51 of the 2D-Array-transducer 50 and its sliding-surface 52. The transducers 40 and 50 are shown with the step-motor-assembly 80 in FIG. 8 from two directions, a bottom-tilted-view (FIG. 8a) and a top-tilted-view (FIG. 8b). The C-Arm Transducer 40 is connected to the circular-track 71 for enabling rotation by the step-motor-assembly 80. The acoustic-stacks 41 and 51 are also shown in FIG. 8a.

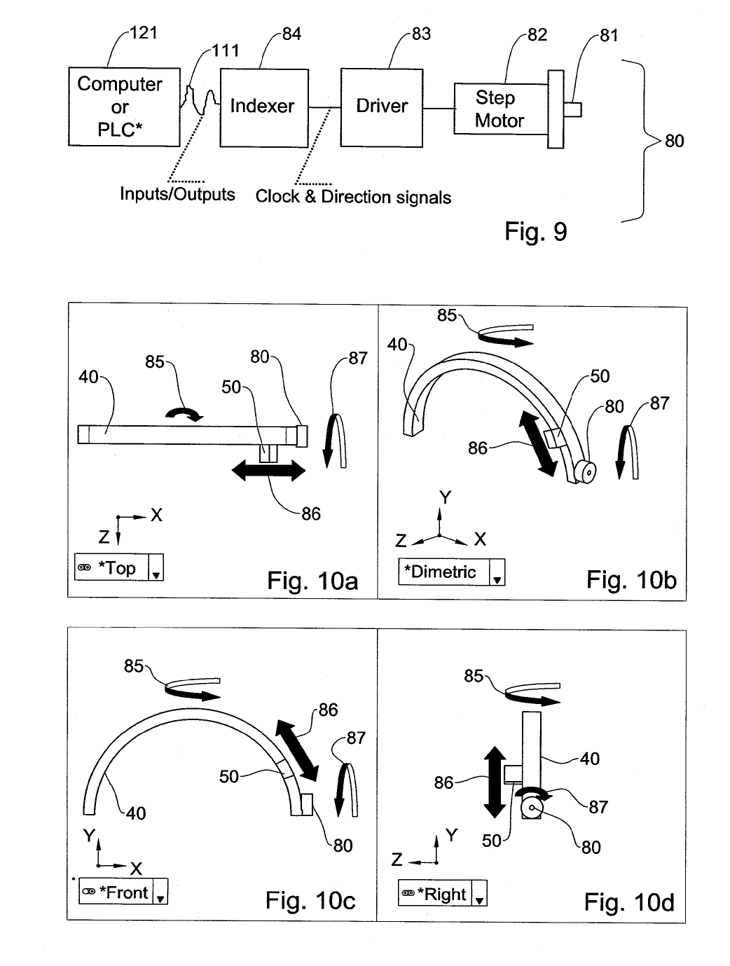

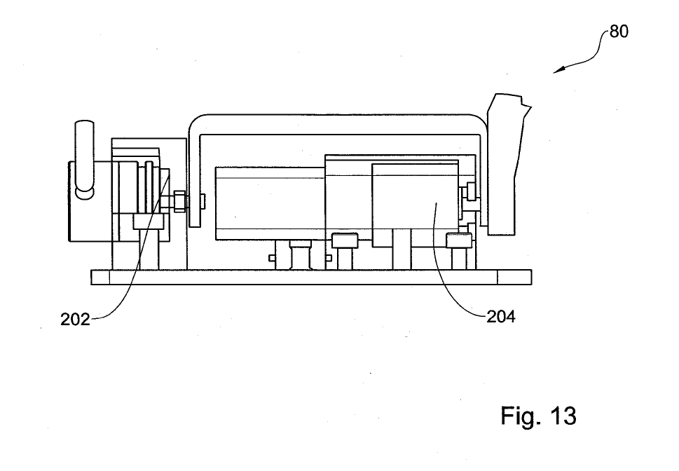

The step-motor-assembly 80 of the scanning-device 30 is controlled from the workstation 120, FIG. 13 shows the step-motor-assembly 80, and FIG. 9 shows schematically the step motor assembly 80. The motor assembly 80 includes a motor 82 having a rotary axis 81. An encoder 202 includes a processor 120, an indexer 84 and a driver 83. The encoder 202 connects directly to the arc axis of the motor 200 in order to reduce or prevent backlash. In the motor assembly 80, the gear axis 204 functions as an arc rotation axis. The driver 83 accepts clock pulses and direction signals and translates these signals into appropriate phase currents in the step-motor 82. The indexer 84 creates the clock pulses and direction signals. The workstation 120 or the processor 121 sends commands to the indexer 84.

The step-motor-assembly 80 drives the two transducers 40 and 50 over the dome 20. In FIG. 10 is shown a set of views that describe the direction of motion driven by the step-motor-assembly 80. A view from above is shown in FIG. 10a, a dimeric view is shown in FIG. 10b a front view is shown in FIG. 10c, and a right side view is shown in FIG. 10d. The rotate-arrow 85 shows the direction of rotation of the C-Arm-tomography-transducer 40 along the circular-track 71 (FIG. 8), the tilt-rotation-arrow 87 shows the tilting rotation of the C-Arm-tomography-transducer 40 and the slide-double-arrow 86 shows the direction the 2D-Array-transducer 50 slides along the C-Arm-tomography-transducer 40. The motions indicated by the arrows 85, 86 and 87 are all driven by the step-motor-assembly 80.

FIG. 12 shows the scanning device and step motor in greater detail. For each planar section of the breast to be imaged, the transducers 40 and 50 are moved along circular arcs. The adaptors 61 are made of an acoustically transparent material such as Aqualene™ to assure that there is no air in between the acoustic stack of the transducers and the dome 20. The plane of the section is not necessarily perpendicular to the axis of the breast. The orientation of the circular arc is monitored by the step motor 200 and is continuously input to the processor 121. The transducers 40 and 50 may act as B-mode ultrasound probes, enabling compound imaging of the images obtained from these transducers. Alternatively, for each pair of a receive transducer and a transmit transducer transmission signals may be measured. The transmission images may be combined with the B-Mode compounded images or with the reflection tomography image produced by an arc of piesoelectric sensors.

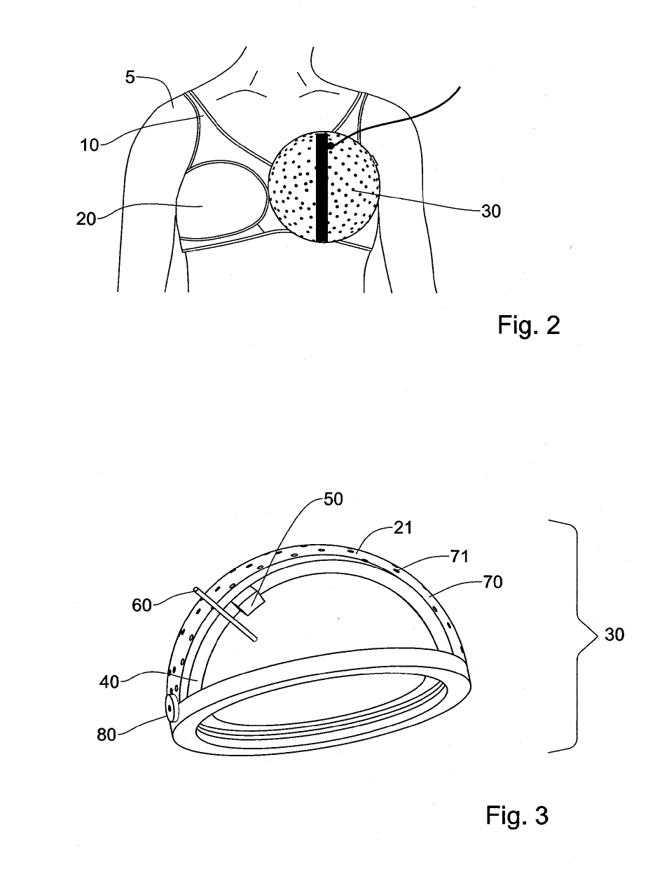

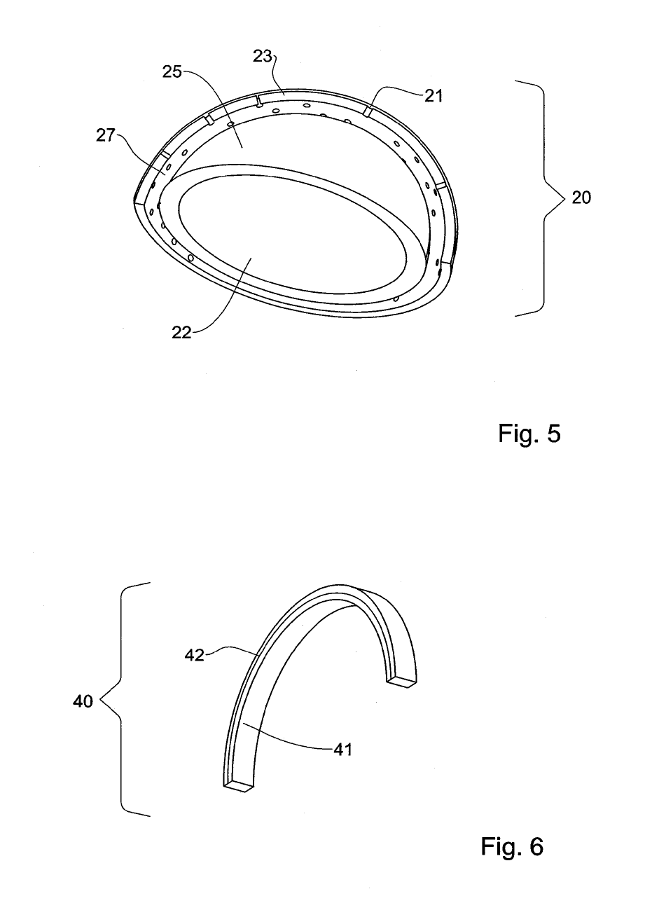

FIG. 2 shows the individual with the scanning device 30 placed on a cup 20 of the bra 10 shown in greater detail in FIG. 5. The cup 20 includes an outer fabric layer 23 and an inner fabric layer 25. Between the inner and outer layers is a thereto-responsive-acoustic-transparent-polymer 27. The state of the thermo-responsive acoustic-transparent polymer 27 is temperature dependent, so that at room temperature it is in a liquid state, while at body temperature (˜37° C.) it is in a solid state. An example such a polymer is the nonionic surfactant polyol, copolymer poloxamer 407 also known as Pluronic F127™. A discussion on the safety of polyol, copolymer poloxamer 407 when in contact with human tissue can be found in Khattak et al [49].

The breast is inserted into the dome 21 with the polymeric material in a viscous form, so that the inner surface of the polymeric material conforms to the shape of the breast surface before solidifying in the shape of the breast surface. The polymeric material may be harder at its outer surface that is in contact with the acoustic stack, as compared to the inner surface that is in contact with the anatomy of the breast. This gradient of hardness along the polymeric material enables producing a perfect outer spherical surface, while keeping the flexibility of adjusting to the complicated surface of the breast. Alternatively, the overlying dome forces the outer surface of the polymeric material to adopt a spherical shape. The polymeric material is an acoustically coupling material which acoustically couples the breast surface with the transducers on the outer surface of the dome. This enables the inner surface of the cup 20 of the bra to conform to the surface of the breast, so that no air is present between the cup and the breast. This allows scanning of the breast with the breast in its natural shape. The thermo-responsive acoustic-transparent polymer 20 may be sterilizable.

In use, breast is inserted into the dome 21 of the scanning device 30. If the bra 10 is being worn, the thermo-responsive-acoustic-transparent-polymer may also be introduced between the bra and the inner surface of the dome 20, so that no air is present between the outer surface of the bra and the inner surface of the dome. Alternatively, if the chair 7 is being used (FIG. 11), then the inverted dome 30 may be filled with the thereto-responsive-acoustic-transparent-polymer before insertion of the breast.

After application of the scanning device 30 to the breast, the transducers 40 and 50 are driven one at a time, and for each driven transducer, each transducer detects ultrasound radiation. The ultrasound wave detected by each transducer is converted by the transducer into an electric signal indicative of the amplitude of the detected wave (ƒ(φᵣ, φₜ) in the case of 2D tomography or ƒ(θᵣ, θₜ, φᵣ, φₜ) in the case of 3D tomography) that is input to the ultrasound system 90 via the cable 100.

The ultrasound system 90 includes a processor configured to generate a 2D or a 3D image from the signals input from the transducers. As explained above, I_{BF}(k) is first calculated. The I_{DT}(k) is then calculated by multiplying by the inverse of the filter g(k) to yield I_{DT}(k)=Õ(k)Π(|k|), which a tomographic image of the breast. This tomographic image may be combined with the compounded images of B-Mode and with the transmission mode images from the transducers 40 and 50. Superimposing these separate types of images is possible due to the fact that these alternative hardware configurations are mechanically coupled to the arc, so that spatial registration is possible.

The 2-D acoustic stack array 51 produces real-time 3-D images (“4-D ultrasound imaging”) for procedure guidance, such as guiding a needle in biopsy or guiding ablation devices. The sliding track 42 and the sliding surface 52 are used for placing the 2-D transducer array in an optimal location with respect to the breast for the guidance procedure. When the 2-D transducer array 50 is operated, the C-arm tomography transducer 40 is kept static. The procedure device, such as the needle 60 in FIG. 3 can be inserted through the needle holes in the cover 70 and through the thermo-responsive acoustic-transparent polymer 20. Mechanical attachment of the 2-D transducer array 50 to the C-arm tomography transducer 40 allows superposition of the real-time 3D images on top of high-resolution tomographic images produced by the C-arm tomography transducer 40.

### ANNEX A

### Difraction Tomography Algorithm for the Limited View Concave Aperture

A new derivation of a two-dimensional DT based on a two-dimensional beamforming (BF) algorithm is discussed, as an alternative approach to standard DT algorithms such as the filtered backpropagation method¹. ¹ Devaney, A. J. 1982, “A filtered backpropagation algorithm for diffraction tomography”, Ultrason. Imaging 4, 336-350.

We assume that the scattering problem is described by a scalar wave field, ψ, solution to

Hψ(r,k₀,\hat{r}₀,ω)=−O(r,ω)ψ(r,k₀\hat{r}₀,ω) (13)

where H is the Helmholtz operator in 2D

$$( \frac{{\partial }^{2}}{\partial {x}^{2}} + \frac{{\partial }^{2}}{\partial {y}^{2}} + {k}_{0}^{2} ) ,$$

k₀ is the background wavenumber (2π/λ), \hat{r}₀ specifies the direction of an incident plane wave that illuminates the object and ω is the angular frequency. The unit vector \hat{r}₀ is defined by the polar angle φₜ.

The object is described by the so-called Object Function that depends on the type of wave field used to probe the object: for electromagnetic wave sensing, it is related to the index of refraction², n(r,ω), through the relation O(r)=k₀²[n²(r,ω)−1], and for acoustic waves, it is linked to the speed of sound and the attenuation coefficient³. In particular, for a lossless object

$$O ( r , ω ) = {k}_{0}^{2} [ {( \frac{{c}_{0}}{c ( r , ω )} )}^{2} - 1 ] \qquad ( 14 )$$

where c₀ is the sound speed of the homogeneous background in which the object is immersed and c(r,ω) is the local sound speed inside the object. The dependence of the object function on ω is because of dispersion and energy dissipation phenomena. The analysis performed in the rest of this section will consider monochromatic wave fields; therefore, the explicit dependence on ω is omitted. ² Born, M. & Wolf, E. 1999 Principles of optics. Cambridge, UK: Cambridge University Press.³ Kak, A. C. & Slaney, M. 1988 Principles of computerized tomographic imaging. New York, N.Y.: IEEE Press.

### Two-Dimensional Beamforming Algorithm Over a Limited View Arc

Let us assume that the scattering amplitude, ƒ(φᵣ, φₜ), can be measured as a continuous function of the illumination and detection directions, φᵣ, φₜε[0,ξ], (note that for a full circle φᵣ, φₜε[0,2π]), these angles corresponding to the angles relative to the x-axis of a standard polar coordinate system. In principle, this could be achieved with the array of transreceivers that partially surrounds the object, placed on a limited view circular arc.

Standard BF produces the image of an object at a point, z, of the image space by focusing an incident beam at r=z in the object space. The resulting scattered field is subsequently phase shifted and integrated over the aperture of the array, so that only the contributions to the scattered field from the focal point are added coherently. This two-step process is obtained by means of the BF functional

ℑ_{BF}=∫₀^{ξ}dφᵣ∫₀^{ξ}dφₜ×exp[−ik₀û(φᵣ)·z]ƒ(φᵣ,φₜ)exp[ik₀û(φₜ)·z] (15)

where û is the unit vector associated with the angle φ. As discussed by⁴ for the full circle two-dimensional case, the second exponential in equation (III) represents focusing in transmission, whereas the first corresponds to the focusing of the received scattered field. The point spread function (PSF) associated with the functional (2) can be obtained by considering the image of a point scatterer at position r. In this case, the free-scattering amplitude is

ƒ_{free}(φᵣ,φₜ)=Nexp{−ik₀[û(φₜ)−û(φᵣ)]·r} (16)

where

$$= \frac{exp ( \frac{i π}{4} )}{\sqrt{8 π {k}_{0}}} ,$$

and the Point Spread function (PSF), also called the Spatial Impulse Response (SIR) reads

h_{BF}(z−r)=N∫₀^{ξ}dφᵣ∫₀^{ξ}dφₜ×exp{−ik₀û(φᵣ)·(z−r)}exp{ik₀û(φₜ)·(z−r)} (17)

û(φᵣ)·(z−r)=|z−r| cos α, where α is the angle between the receive unit vector û(φᵣ) and the vector z−r. Denoting the angle of z−r as φ′, α=φᵣ−φ′. ⁴ Simonetti, F. & Huang, L. 2008, “From beamforming to diffraction tomography”, J. Appl. Phys. 103, 103 110.

The Jacobi-Anger expansion gives:

exp{ik₀|z−r|cos(α)}=J₀(k₀|z−r|)+2Σₙ₌₁^{∞}iⁿJₙ(k₀|z−r|)cos(nα)

where Jₙ is the Bessel function of the order n.

We have⁵:

$${\int }_{0}^{ξ} d {φ}_{r} exp \{ i {k}_{0} \hat{u} ( {φ}_{r} ) \cdot ( z - r ) \} = ξ {J}_{0} ( {k}_{0} \vert z - r \vert ) + 2 {\sum }_{n = 1}^{∞} {i}^{n} {J}_{n} ( {k}_{0} \vert z - r \vert ) {\int }_{0}^{ξ} d {φ}_{r} cos [ n ( {φ}_{r} - {φ}^{′} ) ] == ξ {J}_{0} ( {k}_{0} \vert z - r \vert ) + 2 {\sum }_{n = 1}^{∞} {i}^{n} {J}_{n} ( {k}_{0} \vert z - r \vert ) {\int }_{0}^{ξ} d {φ}_{r} [ cos ( n {φ}^{′} ) + sin ( n {φ}_{r} ) sin ( n {φ}^{′} ) ]$$

$${\int }_{0}^{ξ} d {φ}_{r} cos ( n {φ}_{r} ) = \frac{1}{n} sin ( n {φ}_{r} ) {\vert}_{0}^{ξ} = \frac{1}{n} sin ( n ξ ) ;$$

$${\int }_{0}^{ξ} d {φ}_{r} sin ( n {φ}_{r} ) = - \frac{1}{n} cos ( n {φ}_{r} ) {\vert}_{0}^{ξ} = - \frac{1}{n} [ cos ( n ξ ) - 1 ] = \frac{1}{n} [ 1 - cos ( n ξ ) ]$$

$${\int }_{0}^{ξ} d {φ}_{r} exp \{ i {k}_{0} \hat{u} ( {φ}_{r} ) \cdot [ z - r ] \} == ξ {J}_{0} ( {k}_{0} \vert z - r \vert ) + 2 {\sum }_{n = 1}^{∞} \frac{{i}^{n}}{n} {J}_{n} ( {k}_{0} \vert z - r \vert ) \{ sin ( n ξ ) cos ( n {φ}^{′} ) + [ 1 - cos ( n ξ ) ] sin ( n {φ}^{′} ) \}$$

⁵ sin(A+B)=sin A cos B+cos A sin B; sin(A−B)=sin A cos B−cos A sin B; cos(A+B)=cos A cos B−sin A sin B; cos(A−B)=cos A cos B+sin A sin B http://www.ies.co.jp/math/java/trig/kahote/kahote.html

The complex conjugate result is obtained for the transmit angles. h_{BF}(z−r) is therefore given by

$${h}_{BF} ( z - r ) = {N ( ξ {J}_{0} ( {k}_{0} \vert z - r \vert ) + 2 {\sum }_{n = 1}^{∞} \frac{{i}^{n}}{n} {J}_{n} ( {k}_{0} \vert z - r \vert ) \{ \begin{matrix}sin ( n ξ ) cos ( n {φ}^{′} ) + \\ [ 1 - cos ( n ξ ) ] sin ( n {φ}^{′} )\end{matrix} \} )}^{*} \times ( ξ {J}_{0} ( {k}_{0} \vert z - r \vert ) + 2 {\sum }_{n = 1}^{∞} \frac{{i}^{n}}{n} {J}_{n} ( {k}_{0} \vert z - r \vert ) \{ \begin{matrix}sin ( n ξ ) cos ( n {φ}^{′} ) + \\ [ 1 - cos ( n ξ ) ] sin ( n {φ}^{′} )\end{matrix} \} )$$

Special Cases:

Note that when ξ=2π (i.e. a full-circle), the received and transmitted beams read:

∫₀^{2π}dφᵣexp{ik₀û(φᵣ)·(z−r)}=2πJ₀(k₀|z−r|)

So h_{BF}(|z−r|)=4π²NJ₀²(k₀|z−r|), for ξ=2π

Note also that for ξ=π (i.e. a semi-circle) and φ′=0, or φ′=multiples of π (i.e. a focal point and field point along [or parallel to] the x axis):

∫₀^{π}dφᵣexp{ik₀û(φᵣ)·(z−r)}=πJ₀(k₀|z−r|),φ′=0,or φ′=multiples of π

So h_{BF}(|z−r|)=π²NJ₀²(k₀|z−r|), for ξ=π, and φ′=0, or φ′=multiples of π

We now calculate the two-dimensional Fourier transform H_{BF}(k) of h_{BF}(z−r):

$$\begin{matrix}{H}_{BF} ( k ) = {\int }_{- ∞}^{∞} {d}^{2} {rh}_{BF} ( z - r ) {e}^{i k \cdot [ z - r ]} \\ = {\int }_{- ∞}^{∞} {d}^{2} r {e}^{i k \cdot [ z - r ]} \times \\ N ( ξ {J}_{0} ( {k}_{0} \vert z - r \vert ) + \\ 2 {\sum }_{n = 1}^{∞} \frac{{i}^{n}}{n} {J}_{n} ( {k}_{0} \vert z - r \vert ) \{ sin ( n ξ ) cos ( n {φ}^{′} ) + \\ { [ 1 - cos ( n ξ ) ] sin ( n {φ}^{′} ) \} )}^{*} \times \\ ( ξ {J}_{0} ( {k}_{0} \vert z - r \vert ) + \\ 2 {\sum }_{n = 1}^{∞} \frac{{i}^{n}}{n} {J}_{n} ( {k}_{0} \vert z - r \vert ) \{ sin ( n ξ ) cos ( n {φ}^{′} ) + \\ [ 1 - cos ( n ξ ) ] sin ( n {φ}^{′} ) \} )\end{matrix}$$

Denoting the angle of k as φ, α=φ′−φ; k·[z−r]=|k∥z−r|cos(α), we now use the Jacobi-Anger expansion again with:

$$\begin{matrix}exp \{ - i k \cdot [ z - r ] \} = exp \{ - i \vert k \vert \vert z - r \vert cos ( α ) \} \\ = {[ {J}_{o} ( \vert k \vert \vert z - r \vert ) + 2 {\sum }_{n = 0}^{∞} {i}^{n} {J}_{n} ( \vert k \vert \vert z - r \vert ) cos ( n α ) ]}^{*}\end{matrix}$$

$${H}_{BF} ( k ) = {\int }_{0}^{∞} r d r {\int }_{0}^{2 π} d {{φ}^{′} ( {J}_{0} ( \vert k \vert \vert z - r \vert ) + 2 {\sum }_{{n}_{3} = 1}^{∞} {i}^{{n}_{3}} {J}_{{n}_{3}} ( \vert k \vert \vert z - r \vert ) [ \begin{matrix}cos ( {n}_{3} φ ) cos ( {n}_{3} {φ}^{′} ) + \\ sin ( {n}_{3} φ ) sin ( {n}_{3} {φ}^{′} )\end{matrix} ] )}^{*} \times \times {N ( ξ {J}_{0} ( {k}_{0} \vert z - r \vert ) + 2 {\sum }_{{n}_{2} = 1}^{∞} \frac{{i}^{{n}_{2}}}{{n}_{2}} {J}_{{n}_{2}} ( {k}_{0} \vert z - r \vert ) \{ \begin{matrix}sin ( {n}_{2} ξ ) cos ( {n}_{2} {φ}^{′} ) + \\ [ 1 - cos ( {n}_{2} ξ ) ] sin ( {n}_{2} {φ}^{′} )\end{matrix} \} )}^{*} \times ( ξ {J}_{0} ( {k}_{0} \vert z - r \vert ) + 2 {\sum }_{{n}_{1} = 1}^{∞} \frac{{i}^{{n}_{1}}}{{n}_{1}} {J}_{{n}_{1}} ( {k}_{0} \vert z - r \vert ) \{ \begin{matrix}sin ( {n}_{1} ξ ) cos ( {n}_{1} {φ}^{′} ) + \\ [ 1 - cos ( {n}_{1} ξ ) ] sin ( {n}_{1} {φ}^{′} )\end{matrix} \} )$$

Integration of the angle φ′ is over simple products of sin and cos trigonometric functions and can be worked out easily.

We therefore, now focus on the integral of a product of 3 Bessel functions. Such integrals are available in closed form in the literature⁶. For example:

$${\int }_{0}^{∞} {t}^{1 - {n}_{1}} {J}_{{n}_{1}} ( at ) {J}_{{n}_{2}} ( bt ) {J}_{{n}_{2}} ( ct ) d t = \frac{{( bc )}^{{n}_{1} - 1} {sin}^{{n}_{1} - \frac{1}{2}} ( A )}{{( 2 π )}^{\frac{1}{2}} {a}^{{n}_{1}}} {P}_{{n}_{2} - \frac{1}{2}}^{\frac{1}{2} - {n}_{1}} ( cos A )$$

$$R ( {n}_{1} ) > - \frac{1}{2} , R ( {n}_{2} ) > - \frac{1}{2}$$

⁶ Y. L. Luke, Integrals of Bessel Functions, McGraw-Hill, New York, 1962, p. 331 and 332

If a, b, c are sides of a triangle of area Δ. And A is

$$A = \{ \begin{matrix}0 , & {a}^{2} < {( b - c )}^{2} and {a}^{2} < {( b + c )}^{2} \\ arc cos \frac{{b}^{2} + {c}^{2} - {a}^{2}}{2 bc} , & {( b - c )}^{2} \leq {a}^{2} \leq {( b + c )}^{2} \\ π , & {a}^{2} > {( b - c )}^{2} and {a}^{2} > {( b + c )}^{2}\end{matrix} \\ Δ = \frac{1}{2} bc sin ( A ) , or sin ( A ) = \frac{2 Δ}{bc}$$

P_{υ}^{μ} are legendre functions⁷ of the first kind:

$${P}_{λ}^{μ} ( z ) = {\frac{1}{Γ ( 1 - μ )} [ \frac{1 + z}{1 - z} ]}^{\frac{μ}{2}} {}_{2}^{}{F}{}_{1}^{} ( - λ , λ + 1 , 1 - μ , \frac{1 - z}{2} )$$

$${}_{2}^{}{F}{}_{1}^{} ( a , b , c , z ) = {\sum }_{n = 0}^{∞} \frac{{( a )}_{n} {( b )}_{n}}{{( c )}_{n}} \frac{{z}^{n}}{n !} , \\ provided that c is not 0 , - 1 , - 2 , … , {and \\ ( a )}_{n} = a ( a + 1 ) ( a + 2 ) … ( a + n - 1 ) , {( a )}_{0} = 1$$

⁷ http://en.wikipedia.org/wiki/Legendre_function; http://en.wikipedia.org/wiki/Hypergeometric_function http://en.wikipedia.org/wiki/Gamma_function
provided that c is not 0, −1, −2, . . . , and (a)ₙ=a(a+1)(a+2) . . . (a+n−1), (a)₀=1

For the particular case of the same index of all three Bessel functions:

$$\begin{matrix}{\int }_{0}^{∞} {t}^{1 - n} {J}_{n} ( at ) {J}_{n} ( bt ) {J}_{n} ( ct ) d t = \frac{{2}^{n - 1} {Δ}^{2 n - 1}}{{π ( abc )}^{n} {( \frac{1}{2} )}_{n}} \\ = > {\int }_{0}^{∞} t {J}_{0} ( at ) {J}_{0} ( bt ) {J}_{0} ( ct ) d t \\ = \frac{1}{2 πΔ}\end{matrix}$$

For our case a=b=k₀, c=|k| we get:

$$\begin{matrix}cos A = \frac{{b}^{2} + {c}^{2} - {a}^{2}}{2 bc} \\ = \frac{{k}_{0}^{2} + {\vert k \vert}^{2} - {k}_{0}^{2}}{2 {k}_{0} \vert k \vert} \\ = \frac{\vert k \vert}{2 {k}_{0}}\end{matrix} \\ sin ( A ) = \sqrt{1 - {cos}^{2} A} = \sqrt{1 - \frac{{\vert k \vert}^{2}}{4 {k}_{0}^{2}}} . \\ Therefore \\ Δ = \frac{1}{2} bc sin ( A ) = \frac{1}{2} {k}_{0} \vert k \vert \sqrt{1 - \frac{{\vert k \vert}^{2}}{4 {k}_{0}^{2}}} and \\ {\int }_{0}^{∞} t {J}_{0} ( at ) {J}_{0} ( bt ) {J}_{0} ( ct ) d t = \frac{1}{2 πΔ} = \frac{1}{π {k}_{0} \vert k \vert \sqrt{1 - \frac{{\vert k \vert}^{2}}{4 {k}_{0}^{2}}}} \qquad ( 18 )$$

We therefore arrived at a formula for H_{BF} of the limited view arc. The triangular relation for a, b, c in our case requires that |k|≦2k₀ so we get the low pass filtering:

$${H}_{BF} = g ( k ) Π ( \vert k \vert ) , where Π ( \vert k \vert ) = \{ \begin{matrix}1 & \vert k \vert < 2 {k}_{0} \\ 0 & \vert k \vert > 2 {k}_{0}\end{matrix} \qquad ( 19 )$$

The DT problem consists of reconstructing the function O(r) from a set of scattering experiments. For this purpose, it is convenient to introduce the representation of the object function in the spatial frequency domain, K-space, which is obtained by performing the two-dimensional Fourier transform of O(r)

$$\tilde{O} ( k ) = {\int }_{- ∞}^{∞} {d}^{2} r O ( r ) {e}^{- i k \cdot r} \qquad ( 20 )$$

We now look at the beamforming image:

ℑ_{BF}=∫_{-∞}^{∞}dr₁∫_{-∞}^{∞}dr₂O(r)h(|z−r|) (21)

that in the spatial frequency domain reads

I_{BF}(k)=Õ(k)H_{BF}(k)=g(k)Õ(k)Π(|k|) (22)

While DT over the entire circle leads to the low-pass-filtered image, Õ(k)Π(|k|), the new BF algorithm introduces a distortion that is described by the additional filter g(k). As a result, the DT image can be obtained from the BF image by applying the filter

$$\frac{1}{g ( k )}$$

to the BF image. Again, this is an alternative approach to other DT algorithms⁸. ⁸ Reference of footnote 3

### ANNEX AI

### Integrals

n₁=0, n₂ for the general formula:

$${\int }_{0}^{∞} {t}^{1 - {n}_{1}} {J}_{{n}_{1}} at ) {J}_{{n}_{2}} ( bt ) {J}_{{n}_{2}} ( ct ) d t = \frac{{( bc )}^{{n}_{1} - 1} {sin}^{{n}_{1} - \frac{1}{2}} ( A )}{{( 2 π )}^{\frac{1}{2}} {a}^{{n}_{1}}} {P}_{{n}_{2} - \frac{1}{2}}^{\frac{1}{2} - {n}_{1}} ( cos A )$$

For the special case n₁=n₂=n₃=0 it reads:

$${\int }_{0}^{∞} {t}^{1} {J}_{0} ( at ) {J}_{{n}_{2}} ( bt ) {J}_{{n}_{2}} ( ct ) d t = \frac{{( bc )}^{- 1} {sin}^{- \frac{1}{2}} ( A )}{{( 2 π )}^{\frac{1}{2}}} {P}_{{n}_{2} - \frac{1}{2}}^{\frac{1}{2}} ( cos A )$$

For our case a=b=k₀, c=|k| we get:

$$\begin{matrix}cos A = \frac{{b}^{2} + {c}^{2} - {a}^{2}}{2 bc} \\ = \frac{{k}_{0}^{2} + {\vert k \vert}^{2} - {k}_{0}^{2}}{2 {k}_{0} \vert k \vert} \\ = \frac{\vert k \vert}{2 {k}_{0}} \\ = z\end{matrix}$$

$$sin ( A ) = \sqrt{1 - {cos}^{2} A} = \sqrt{1 - {z}^{2}} = \sqrt{1 - \frac{{\vert k \vert}^{2}}{4 {k}_{0}^{2}}} . \\ Therefore$$

$$Δ = \frac{1}{2} bc sin ( A ) = \frac{1}{2} {k}_{0} \vert k \vert \sqrt{1 - \frac{{\vert k \vert}^{2}}{4 {k}_{0}^{2}}}$$

$$\begin{matrix}{sin}^{- \frac{1}{2}} ( A ) = {( \sqrt{1 - {z}^{2}} )}^{- \frac{1}{2}} \\ = {[ ( 1 + z ) ( 1 - z ) ]}^{- \frac{1}{4}} \\ = > {\int }_{0}^{∞} t {J}_{0} ( {k}_{0} t ) {J}_{{n}_{2}} ( {k}_{0} t ) {J}_{{n}_{2}} ( \vert k \vert t ) d t \\ = \frac{1}{{k}_{0} \vert k \vert {{( 2 π )}^{\frac{1}{2}} [ ( 1 + z ) ( 1 - z ) ]}^{\frac{1}{4}}} {P}_{{n}_{2} - \frac{1}{2}}^{\frac{1}{2}} ( z )\end{matrix}$$

We now look at the definition of the Legendre Functions:

$${P}_{λ}^{μ} ( z ) = {\frac{1}{Γ ( 1 - μ )} [ \frac{1 + z}{1 - z} ]}^{\frac{μ}{2}} {}_{2}^{}{F}{}_{1}^{} ( - λ , λ + 1 , 1 - μ , \frac{1 - z}{2} )$$

$$Using Γ ( \frac{1}{2} ) = \sqrt{π} we get for μ = \frac{1}{2} and λ = {n}_{2} - \frac{1}{2} : \\ {P}_{{n}_{2} - \frac{1}{2}}^{\frac{1}{2}} ( z ) = {\frac{1}{\sqrt{π}} [ \frac{1 + z}{1 - z} ]}^{\frac{1}{4}} {}_{2}^{}{F}{}_{1}^{} ( - {n}_{2} + \frac{1}{2} , {n}_{2} - \frac{1}{2} + 1 , \frac{1}{2} , \frac{1 - z}{2} )$$

$$\begin{matrix}{\int }_{0}^{∞} t {J}_{0} ( {k}_{0} t ) {J}_{{n}_{2}} ( {k}_{0} t ) {J}_{{n}_{2}} ( \vert k \vert t ) d t = \frac{1}{{k}_{0} \vert k \vert {{( 2 π )}^{\frac{1}{2}} [ ( 1 + z ) ( 1 - z ) ]}^{\frac{1}{4}}} \\ {\frac{1}{\sqrt{π}} [ \frac{1 + z}{1 - z} ]}^{\frac{1}{4}} {}_{2}^{}{F}{}_{1}^{} \\ ( \begin{matrix}- {n}_{2} + \frac{1}{2} , \\ {n}_{2} - \frac{1}{2} + 1 , \frac{1}{2} , \frac{1 - z}{2}\end{matrix} ) \\ = \frac{1}{{k}_{0} \vert k \vert \sqrt{2} π \sqrt{1 - z}} {}_{2}^{}{F}{}_{1}^{} \\ ( - {n}_{2} + \frac{1}{2} , {n}_{2} + \frac{1}{2} , \frac{1}{2} , \frac{1 - z}{2} )\end{matrix}$$

$$( {}_{2}^{}{F}{}_{1}^{} ( \frac{1}{2} , \frac{1}{2} ; \frac{1}{2} ; z ) = \frac{1}{\sqrt{1 - z}} )$$

$${}_{2}^{}{F}{}_{1}^{} ( \frac{1}{2} , \frac{1}{2} , \frac{1}{2} , y ) = \frac{1}{\sqrt{1 - y}} ;$$

$$\begin{matrix}{ }_{2} {F}_{1} ( \frac{1}{2} , \frac{1}{2} , \frac{1}{2} , \frac{1 - z}{2} ) = \frac{1}{\sqrt{1 - \frac{1 - z}{2}}} \\ = \frac{\sqrt{2}}{\sqrt{2 - ( 1 - z )}} \\ = \frac{\sqrt{2}}{\sqrt{1 + z}}\end{matrix}$$

$$\begin{matrix}= {>}_{2} {F}_{1} ( \frac{1}{2} , \frac{1}{2} , \frac{1}{2} , \frac{1 - z}{2} ) = \frac{1}{\sqrt{1 - \frac{1 - z}{2}}} \\ = \frac{\sqrt{2}}{\sqrt{2 - ( 1 - z )}} \\ = \frac{\sqrt{2}}{\sqrt{1 + z}}\end{matrix}$$

$$\begin{matrix}{\int }_{0}^{∞} t {J}_{0} ( {k}_{0} t ) {J}_{0} ( {k}_{0} t ) {J}_{0} ( \vert k \vert t ) d t = \frac{1}{{k}_{0} \vert k \vert \sqrt{2} π \sqrt{1 - z}} {}_{2}^{}{F}{}_{1}^{} \\ ( \frac{1}{2} , \frac{1}{2} , \frac{1}{2} , \frac{1 - z}{2} ) \\ = \frac{1}{{k}_{0} \vert k \vert \sqrt{2} π \sqrt{1 - z}} \frac{\sqrt{2}}{\sqrt{1 + z}} \\ = \frac{1}{{k}_{0} \vert k \vert π \sqrt{1 - z}} \frac{1}{\sqrt{1 + z}} \\ = \frac{1}{{k}_{0} \vert k \vert π \sqrt{[ ( 1 - {z}^{2} ) ]}} \\ = \frac{1}{{k}_{0} \vert k \vert π \sqrt{1 - \frac{{\vert k \vert}^{2}}{4 {k}_{0}^{2}}}}\end{matrix}$$

For n₂=0 the Gauss Hypergeometric Function is found in⁹

$${(}_{2} {F}_{1} ( \frac{1}{2} , \frac{1}{2} : \frac{1}{2} : z ) = \frac{1}{\sqrt{1 - z}} ) {}_{2}^{}{F}{}_{1}^{} ( \frac{1}{2} , \frac{1}{2} , \frac{1}{2} , y ) = \frac{1}{\sqrt{1 - z}} ; = > {}_{2}^{}{F}{}_{1}^{} ( \frac{1}{2} , \frac{1}{2} , \frac{1}{2} , \frac{1 - z}{2} ) = \frac{1}{\sqrt{1 - \frac{1 - z}{2}}} = \frac{\sqrt{2}}{\sqrt{2 - ( 1 - z )}} = \frac{\sqrt{2}}{\sqrt{1 + z}} {\int }_{0}^{∞} t {J}_{0} ( {k}_{0} t ) {J}_{0} ( {k}_{0} t ) {J}_{0} ( \vert k \vert t ) d t = \frac{1}{{k}_{0} \vert k \vert \sqrt{2} π \sqrt{1 - z}} {}_{2}^{}{F}{}_{1}^{} ( \frac{1}{2} , \frac{1}{2} , \frac{1}{2} , \frac{1 - z}{2} ) = \frac{1}{{k}_{0} \vert k \vert \sqrt{2} π \sqrt{1 - z}} \frac{\sqrt{2}}{\sqrt{1 + z}} = \frac{1}{{k}_{0} \vert k \vert π \sqrt{1 - z}} \frac{1}{\sqrt{1 + z )}} = \frac{1}{{k}_{0} \vert k \vert π \sqrt{[ ( 1 - {z}^{2} ) ]}} = \frac{1}{{k}_{0} \vert k \vert \sqrt{1 - \frac{{\vert k \vert}^{2}}{4 {k}_{0}^{2}}}}$$

⁹ http://functions.wolfram.com/HypergeometricFunctions/Hypergeometric2F1/03/07/07/01/
we used

$$z = \frac{\vert k \vert}{2 {k}_{0}} .$$

n₂=1

The above Gauss Hypergeometric Function is

$$( {}_{2}^{}{F}{}_{1}^{} ( - \frac{1}{2} , \frac{3}{2} ; \frac{1}{2} ; z ) = \frac{1 - 2 z}{\sqrt{1 - z}} )$$

$${ }_{2} {F}_{1} ( - \frac{1}{2} , \frac{3}{2} , \frac{1}{2} , y ) = \frac{1 - 2 y}{\sqrt{1 - y}} ;$$

$$\text{=>} { }_{2} {F}_{1} ( - \frac{1}{2} , \frac{3}{2} , \frac{1}{2} , \frac{1 - z}{2} ) = \frac{z}{\sqrt{1 - \frac{1 - z}{2}}} = \frac{\sqrt{2} z}{\sqrt{2 - ( 1 - z )}} = \frac{\sqrt{2} z}{\sqrt{1 + z}}$$

$${\int }_{0}^{∞} t {J}_{0} ( {k}_{0} t ) {J}_{1} ( {k}_{0} t ) {J}_{1} ( \vert k \vert t ) d t = \frac{1}{{k}_{0} \vert k \vert \sqrt{2} π \sqrt{1 - z}} {}_{2}^{}{F}{}_{1}^{} ( - \frac{1}{2} , \frac{3}{2} , \frac{1}{2} , \frac{1 - z}{2} ) = \frac{1}{{k}_{0} \vert k \vert \sqrt{2} π \sqrt{1 - z}} \frac{\sqrt{2} z}{\sqrt{1 + z}} == \frac{z}{{k}_{0} \vert k \vert π \sqrt{1 - {z}^{2}}} = \frac{\frac{\vert k \vert}{2 {k}_{0}}}{{k}_{0} \vert k \vert π \sqrt{1 - \frac{{\vert k \vert}^{2}}{4 {k}_{0}^{2}}}} = \frac{1}{2 {k}_{0}^{2} π \sqrt{1 - \frac{{\vert k \vert}^{2}}{4 {k}_{0}^{2}}}}$$

we used

$$z = \frac{\vert k \vert}{2 {k}_{0}} .$$

n₂=2

The above Gauss Hypergeometric Function is

$$( {}_{2}^{}{F}{}_{1}^{} ( - \frac{3}{2} , \frac{5}{2} ; \frac{1}{2} ; z ) = \frac{8 ( z - 3 ) z + 1}{\sqrt{1 - z}} )$$

$${ }_{2} {F}_{1} ( - \frac{3}{2} , \frac{5}{2} , \frac{1}{2} , y ) = \frac{8 ( y - 1 ) y + 1}{\sqrt{1 - y}} ;$$

$$\text{=>} {}_{2}^{}{F}{}_{1}^{} ( - \frac{3}{2} , \frac{5}{2} , \frac{1}{2} , \frac{1 - z}{2} ) = \frac{8 ( \frac{1 - z}{2} - 1 ) \frac{1 - z}{2} + 1}{\sqrt{1 - \frac{1 - z}{2}}} = \frac{- 2 ( z + 1 ) ( 1 - z ) + 1}{\sqrt{1 - \frac{( 1 - z )}{2}}} = \frac{\sqrt{2} [ 1 - 2 ( z + 1 ) ( 1 - z ) ]}{\sqrt{1 + z}} = \frac{\sqrt{2} [ 1 + 2 ( {z}^{2} - 1 ) ]}{\sqrt{1 + z}} = \frac{\sqrt{2} ( 2 {z}^{2} - 1 )}{\sqrt{1 + z}}$$

$${\int }_{0}^{∞} t {J}_{0} ( {k}_{0} t ) {J}_{2} ( {k}_{0} t ) {J}_{2} ( \vert k \vert t ) d t = \frac{1}{{k}_{0} \vert k \vert \sqrt{2} π \sqrt{1 - z}} {}_{2}^{}{F}{}_{1}^{} ( - \frac{3}{2} , \frac{5}{2} , \frac{1}{2} , \frac{1 - z}{2} ) = \frac{1}{{k}_{0} \vert k \vert \sqrt{2} π \sqrt{1 - z}} \frac{\sqrt{2} ( 2 {z}^{2} - 1 )}{\sqrt{1 + z}} = \frac{2 {z}^{2} - 1}{{k}_{0} \vert k \vert π \sqrt{1 - {z}^{2}}} = \frac{2 {z}^{2} - 1}{{k}_{0} \vert k \vert π \sqrt{1 - {z}^{2}}} = \frac{\frac{{\vert k \vert}^{2}}{2 {k}_{0}^{2}} - 1}{{k}_{0} \vert k \vert π \sqrt{1 - \frac{{\vert k \vert}^{2}}{4 {k}_{0}^{2}}}}$$

we used

$$z = \frac{\vert k \vert}{2 {k}_{0}} .$$

n₂=3

The above Gauss Hypergeometric Function is

$$( {}_{2}^{}{F}{}_{1}^{} ( - \frac{5}{2} , \frac{7}{2} ; \frac{1}{2} ; z ) = \frac{1 - 2 {( 3 - 4 z )}^{2} z}{\sqrt{1 - z}} )$$

$${}_{2}^{}{F}{}_{1}^{} ( - \frac{5}{2} , \frac{7}{2} , \frac{1}{2} , y ) = \frac{1 - 2 {( 3 - 4 y )}^{2} y}{\sqrt{1 - y}} \text{=>} {}_{ 2}^{}{F}{}_{1}^{} ( - \frac{5}{2} , \frac{7}{2} , \frac{1}{2} , \frac{1 - z}{2} ) = \frac{1 - 2 {( 3 - 4 \frac{1 - z}{2} )}^{2} \frac{1 - z}{2}}{\sqrt{1 - \frac{1 - z}{2}}} == \frac{\sqrt{2} ( 1 - {( 1 + 2 z )}^{2} ( 1 - z ) )}{\sqrt{1 + z}}$$

We get:

$$\begin{matrix}{\int }_{0}^{∞} t {J}_{0} ( {k}_{0} t ) {J}_{3} ( {k}_{0} t ) {J}_{3} ( \vert k \vert t ) d t = \frac{1}{{k}_{0} \vert k \vert \sqrt{2} π \sqrt{1 - z}} {}_{ 2}^{}{F}{}_{1}^{} ( - \frac{5}{2} , \frac{7}{2} , \frac{1}{2} , \frac{1 - z}{2} ) \\ = \frac{1}{{k}_{0} \vert k \vert \sqrt{2} π \sqrt{1 - z}} \frac{\sqrt{2} ( 1 - {( 1 + 2 z )}^{2} ( 1 - z ) )}{\sqrt{1 + z}} \\ = \frac{1 - {( 1 + 2 z )}^{2} ( 1 - z )}{{k}_{0} \vert k \vert π \sqrt{1 - {z}^{2}}} \\ = \frac{1 - {( 1 + \frac{\vert k \vert}{{k}_{0}} )}^{2} ( 1 - \frac{\vert k \vert}{2 {k}_{0}} )}{{k}_{0} \vert k \vert π \sqrt{1 - \frac{{\vert k \vert}^{2}}{4 {k}_{0}^{2}}}}\end{matrix}$$

we used

$$z = \frac{\vert k \vert}{2 {k}_{0}} .$$

### Annex A II

### Integration to obtain H_{BF}(k)

h_{BF}(z−r)=N∫₀^{ξ}dφᵣ∫₀^{ξ}dφₜ×exp{−ik₀û(φᵣ)·(z−r)}exp{ik₀û(φₜ)·(z−r)}

Bessel Functions¹⁰ and Jacobi Anger Expansion¹¹:

$$\begin{matrix}〈 r - z \vert {k}_{0} 〉 = exp [ {k}_{0} \cdot ( r - z ) ] \\ = exp \{ i {k}_{0} \vert z - r \vert cos ( α ) \} \\ = {\sum }_{n = - ∞}^{∞} {i}^{n} {J}_{n} ( {k}_{0} \vert z - r \vert ) exp ( i n α ) \\ = {\sum }_{n = - ∞}^{∞} {J}_{n} ( {k}_{0} \vert z - r \vert ) exp [ i n ( α + \frac{π}{2} ) ] \\ = {J}_{0} ( {k}_{0} \vert z - r \vert ) + {\sum }_{n = - ∞ , n \neq 0}^{∞} {J}_{n} ( {k}_{0} \vert z - r \vert ) exp [ i n ( α + \frac{π}{2} ) ]\end{matrix}$$

$$α = {φ}_{r , t} - {φ}^{′} .$$

Integration over φᵣ:

$${\int }_{0}^{ξ} d {φ}_{r} exp \{ - i {k}_{0} \hat{u} ( {φ}_{r} ) \cdot ( z - r ) \} = ξ {J}_{0} ( {k}_{0} \vert z - r \vert ) + {\int }_{0}^{ξ} d {φ}_{r} {{\sum }_{n = - ∞ , n \neq 0}}^{∞} {J}_{n} ( {k}_{0} \vert z - r \vert ) exp [ - i n ( α + \frac{π}{2} ) ] = ξ {J}_{0} ( {k}_{0} \vert z - r \vert ) + {\int }_{0}^{ξ} d {φ}_{r} {{\sum }_{n = - ∞ , n \neq 0}}^{∞} {J}_{n} ( {k}_{0} \vert z - r \vert ) exp [ - i n ( {φ}_{r} - {φ}^{′} + \frac{π}{2} ) ] = ξ {J}_{0} ( {k}_{0} \vert z - r \vert ) + {{\sum }_{n = - ∞ , n \neq 0}}^{∞} {J}_{n} ( {k}_{0} \vert z - r \vert ) {\int }_{0}^{ξ} d {φ}_{r} exp [ - i n ( {φ}_{r} - {φ}^{′} + \frac{π}{2} ) ] = ξ {J}_{0} ( {k}_{0} \vert z - r \vert ) + {{\sum }_{n = - ∞ , n \neq 0}}^{∞} exp [ - i n ( - {φ}^{′} + \frac{π}{2} ) ] {J}_{n} ( {k}_{0} \vert z - r \vert ) {\int }_{0}^{ξ} d {φ}_{r} exp [ - i n {φ}_{r} ] = ξ {J}_{0} ( {k}_{0} \vert z - r \vert ) + {{\sum }_{n = - ∞ , n \neq 0}}^{∞} {e}^{i n ( {φ}^{′} - \frac{π}{2} )} {J}_{n} ( {k}_{0} \vert z - r \vert ) \frac{{e}^{- i n ξ} - 1}{- n {e}^{i \frac{π}{2}}} = ξ {J}_{0} ( {k}_{0} \vert z - r \vert ) + {{\sum }_{n = - ∞ , n \neq 0}}^{∞} \frac{{e}^{- i ( 1 + n ) \frac{π}{2} [ {e}^{- i n ξ} - 1 ]}}{- n} {J}_{n} ( {k}_{0} \vert z - r \vert ) {e}^{i n {φ}^{′}}$$

¹⁰ http://en.wikipedia.org/wiki/Bessel_function¹¹ http://en.wikipedia.org/wiki/Jacobi-Anger_expansion

Integration over φₜ:

$${\int }_{0}^{ξ} d {φ}_{t} exp \{ i {k}_{0} \hat{u} ( {φ}_{r} ) \cdot ( z - r ) \} = ξ {J}_{0} ( {k}_{0} \vert z - r \vert ) + {\int }_{0}^{ξ} d {φ}_{t} {{\sum }_{n = - ∞ , n \neq 0}}^{∞} {J}_{n} ( {k}_{0} \vert z - r \vert ) exp [ i n ( α + \frac{π}{2} ) ] = ξ {J}_{0} ( {k}_{0} \vert z - r \vert ) + {\int }_{0}^{ξ} d {φ}_{t} {{\sum }_{n = - ∞ , n \neq 0}}^{∞} {J}_{n} ( {k}_{0} \vert z - r \vert ) exp [ i n ( {φ}_{t} - {φ}^{′} + \frac{π}{2} ) ] = ξ {J}_{0} ( {k}_{0} \vert z - r \vert ) + {{\sum }_{n = - ∞ , n \neq 0}}^{∞} {J}_{n} ( {k}_{0} \vert z - r \vert ) {\int }_{0}^{ξ} d {φ}_{t} exp [ i n ( {φ}_{t} - {φ}^{′} + \frac{π}{2} ) ] = ξ {J}_{0} ( {k}_{0} \vert z - r \vert ) + {{\sum }_{n = - ∞ , n \neq 0}}^{∞} exp [ i n ( - {φ}^{′} + \frac{π}{2} ) ] {J}_{n} ( {k}_{0} \vert z - r \vert ) {\int }_{0}^{ξ} d {φ}_{t} exp [ i n {φ}_{t} ] = ξ {J}_{0} ( {k}_{0} \vert z - r \vert ) + {{\sum }_{n = - ∞ , n \neq 0}}^{∞} {e}^{i n ( - {φ}^{′} + \frac{π}{2} )} {J}_{n} ( {k}_{0} \vert z - r \vert ) \frac{{e}^{i n ξ} - 1}{i n} = ξ {J}_{0} ( {k}_{0} \vert z - r \vert ) + {{\sum }_{n = - ∞ , n \neq 0}}^{∞} {e}^{i n ( - {φ}^{′} + \frac{π}{2} )} {J}_{n} ( {k}_{0} \vert z - r \vert ) \frac{{e}^{i n ξ} - 1}{i n } \frac{i}{i} = ξ {J}_{0} ( {k}_{0} \vert z - r \vert ) + {{\sum }_{n = - ∞ , n \neq 0}}^{∞} {e}^{i \frac{π}{2}} {e}^{i n ( - {φ}^{′} + \frac{π}{2} )} {J}_{n} ( {k}_{0} \vert z - r \vert ) \frac{{e}^{i n ξ} - 1}{- n } = ξ {J}_{0} ( {k}_{0} \vert z - r \vert ) + {\sum }_{n = - ∞ , n \neq 0}^{∞} {J}_{n} ( {k}_{0} \vert z - r \vert ) \frac{{e}^{i ( 1 + n ) \frac{π}{2} [ {e}^{i n ξ} - 1 ]}}{- n} {e}^{- i n {φ}^{′}}$$

Indeed the above integration over φₜ is the complex conjugate of the integration over φᵣ.

$$⇒ \\ {h}_{BF} ( z - r ) = N \{ ξ {J}_{0} ( {k}_{0} \vert z - r \vert ) + {\sum }_{{n}_{2} = - ∞ , {n}_{2} \neq 0}^{∞} \frac{{e}^{- i ( 1 + {n}_{2} ) \frac{π}{2}} [ 1 - {e}^{- i {n}_{2} ξ} ]}{{n}_{2}} {J}_{{n}_{2}} ( {k}_{0} \vert z - r \vert ) {e}^{i {n}_{2} {φ}^{′}} \} \{ ξ {J}_{0} ( {k}_{0} \vert z - r \vert ) + {\sum }_{{n}_{1} = - ∞ , {n}_{1} \neq 0}^{∞} \frac{{e}^{i ( 1 + {n}_{1} ) \frac{π}{2}} [ 1 - {e}^{i {n}_{1} ξ} ]}{{n}_{1}} {J}_{{n}_{1}} ( {k}_{0} \vert z - r \vert ) {e}^{- i {n}_{1} {φ}^{′}} \}$$

Fourier transforming:

$$\begin{matrix}\begin{matrix}{H}_{BF} ( k ) = \int {d}^{2} ( z - r ) {h}_{BF} ( z - r ) {e}^{- i k \cdot [ z - r ]} \\ = \int {d}^{2} ( z - r ) {e}^{- i k \cdot [ z - r ]} \times \times \\ N \{ \begin{matrix}ξ {J}_{0} ( {k}_{0} \vert z - r \vert ) + \\ {\sum }_{{n}_{2} = - ∞ , {n}_{2} \neq 0}^{∞} \frac{{e}^{- i ( 1 + {n}_{2} ) \frac{π}{2}} [ 1 - {e}^{- i {n}_{2} ξ} ]}{{n}_{2}} {J}_{{n}_{2}} ( {k}_{0} \vert z - r \vert ) {e}^{i {n}_{2} {φ}^{′}}\end{matrix} \} \\ \{ \begin{matrix}ξ {J}_{0} ( {k}_{0} \vert z - r \vert ) + \\ {\sum }_{{n}_{1} = - ∞ , {n}_{1} \neq 0}^{∞} \frac{{e}^{i ( 1 + {n}_{1} ) \frac{π}{2}} [ 1 - {e}^{i {n}_{1} ξ} ]}{{n}_{1}} {J}_{{n}_{1}} ( {k}_{0} \vert z - r \vert ) {e}^{- i {n}_{1} {φ}^{′}}\end{matrix} \}\end{matrix} & \\ \begin{matrix}{H}_{BF} ( k ) = \int {d}^{2} ( z - r ) {h}_{BF} ( z - r ) {e}^{- i k \cdot [ z - r ]} \\ = \int {d}^{2} ( z - r ) {e}^{- i k \cdot [ z - r ]} \times \times \\ N \{ \begin{matrix}{ξ }^{2} {J}_{0} ( {k}_{0} \vert z - r \vert ) {J}_{0} ( {k}_{0} \vert z - r \vert ) + ξ {J}_{0} ( {k}_{0} \vert z - r \vert ) \\ {\sum }_{{n}_{2} = - ∞ , {n}_{2} \neq 0}^{∞} \frac{{e}^{- i ( 1 + {n}_{2} ) \frac{π}{2}} [ 1 - {e}^{- i {n}_{2} ξ} ]}{{n}_{2}} \\ {J}_{{n}_{2}} ( {k}_{0} \vert z - r \vert ) {e}^{i {n}_{2} {φ}^{′}} + ξ {J}_{0} ( {k}_{0} \vert z - r \vert ) \\ {\sum }_{{n}_{1} = - ∞ , {n}_{1} \neq 0}^{∞} \frac{{e}^{i ( 1 + {n}_{1} ) \frac{π}{2}} [ 1 - {e}^{i {n}_{1} ξ} ]}{{n}_{1}} {J}_{{n}_{1}} ( {k}_{0} \vert z - r \vert ) {e}^{- i {n}_{1} {φ}^{′}} + \\ [ {\sum }_{{n}_{2} = - ∞ , {n}_{2} \neq 0}^{∞} \frac{{e}^{- i ( 1 + {n}_{2} ) \frac{π}{2}} [ 1 - {e}^{- i {n}_{2} ξ} ]}{{n}_{2}} {J}_{{n}_{2}} ( {k}_{0} \vert z - r \vert ) {e}^{i {n}_{2} {φ}^{′}} ] \times \\ [ {\sum }_{{n}_{1} = - ∞ , {n}_{1} \neq 0}^{∞} \frac{{e}^{i ( 1 + {n}_{1} ) \frac{π}{2}} [ 1 - {e}^{i {n}_{1} ξ} ]}{{n}_{1}} {J}_{{n}_{1}} ( {k}_{0} \vert z - r \vert ) {e}^{- i {n}_{1} {φ}^{′}} ]\end{matrix} \} \\ ≡ \int {d}^{2} ( z - r ) {e}^{- i k \cdot [ z - r ]} \{ {L}_{0} + {L}_{1} + {L}_{2} + {L}_{3} \}\end{matrix} & \end{matrix}$$

The above expression is marked with intermediate terms for convenience.

Inspect in L₁ the summation in two steps:

$${\sum }_{{n}_{2} = - ∞ , {n}_{2} \neq 0}^{∞} \frac{{e}^{- i ( 1 + {n}_{2} ) \frac{π}{2}} [ 1 - {e}^{- i {n}_{2} ξ} ]}{{n}_{2}} {J}_{{n}_{2}} ( {k}_{0} \vert z - r \vert ) {e}^{i {n}_{2} {φ}^{′}} = {\sum }_{{n}_{2} = 1}^{∞} \frac{{e}^{- i ( 1 + {n}_{2} ) \frac{π}{2}} [ 1 - {e}^{- i {n}_{2} ξ} ]}{{n}_{2}} {J}_{{n}_{2}} ( {k}_{0} \vert z - r \vert ) {e}^{i {n}_{2} {φ}^{′}} + {\sum }_{{n}_{2} = - ∞ ,}^{- 1} \frac{{e}^{- i ( 1 + {n}_{2} ) \frac{π}{2}} [ 1 - {e}^{- i {n}_{2} ξ} ]}{{n}_{2}} {J}_{{n}_{2}} ( {k}_{0} \vert z - r \vert ) {e}^{i {n}_{2} {φ}^{′}} == {\sum }_{{n}_{2} = 1}^{∞} \frac{{e}^{- i ( 1 + {n}_{2} ) \frac{π}{2}} [ 1 - {e}^{- i {n}_{2} ξ} ]}{{n}_{2}} {J}_{{n}_{2}} ( {k}_{0} \vert z - r \vert ) {e}^{i {n}_{2} {φ}^{′}} + {\sum }_{{n}_{2} = 1}^{∞} \frac{{e}^{- i ( 1 + {n}_{2} ) \frac{π}{2}} [ {e}^{i {n}_{2} ξ} - 1 ]}{{n}_{2}} {J}_{- {n}_{2}} ( {k}_{0} \vert z - r \vert ) {e}^{- i {n}_{2} {φ}^{′}} = {\sum }_{{n}_{2} = 1}^{∞} \frac{{e}^{- i ( 1 + {n}_{2} ) \frac{π}{2}} [ 1 - {e}^{- i {n}_{2} ξ} ]}{{n}_{2}} {J}_{{n}_{2}} ( {k}_{0} \vert z - r \vert ) {e}^{i {n}_{2} {φ}^{′}} + \frac{{e}^{- i ( 1 - {n}_{2} ) \frac{π}{2}} [ {e}^{i {n}_{2} ξ} - 1 ]}{{n}_{2}} {J}_{- {n}_{2}} ( {k}_{0} \vert z - r \vert ) {e}^{- i {n}_{2} {φ}^{′}}$$

Using: J₋ₙ_{₂}(k₀|z−r|)=(−1)ⁿ^{₂}Jₙ_{₂}(k₀|z−r|) we get:

$$= {\sum }_{{n}_{2} = 1}^{∞} \frac{{e}^{- i ( 1 + {n}_{2} ) \frac{π}{2}} [ 1 - {e}^{- i {n}_{2} ξ} ]}{{n}_{2}} {J}_{{n}_{2}} ( {k}_{0} \vert z - r \vert ) {e}^{i {n}_{2} {φ}^{′}} + \frac{{e}^{- i ( 1 - {n}_{2} ) \frac{π}{2}} [ {e}^{i {n}_{2} ξ} - 1 ]}{{n}_{2}} {( - 1 )}^{{n}_{2}} {J}_{{n}_{2}} ( {k}_{0} \vert z - r \vert ) {e}^{- i {n}_{2} {φ}^{′}}$$

$${L}_{1} = N ξ {J}_{0} ( {k}_{0} \vert z - r \vert ) {\sum }_{{n}_{2} = 1}^{∞} \frac{1}{{n}_{2}} {e}^{- i \frac{π}{2}} {J}_{{n}_{2}} ( {k}_{0} \vert z - r \vert ) \{ {e}^{- i {n}_{2} \frac{π}{2}} [ 1 - {e}^{- i {n}_{2} ξ} ] {e}^{i {n}_{2} {φ}^{′}} + {e}^{i {n}_{2} \frac{π}{2}} [ {e}^{i {n}_{2} ξ} - 1 ] {( - 1 )}^{{n}_{2}} {e}^{- i {n}_{2} {φ}^{′}} \}$$

Similarly inspect L₂ for a summation over n₁ in two steps:

$${\sum }_{{n}_{1} = - ∞ , {n}_{1} \neq 0}^{∞} \frac{{e}^{i ( 1 + {n}_{1} ) \frac{π}{2}} [ 1 - {e}^{i {n}_{1} ξ} ]}{{n}_{1}} {J}_{{n}_{1}} ( {k}_{0} \vert z - r \vert ) {e}^{- i {n}_{1} {φ}^{′}} == {\sum }_{{n}_{1} = 1}^{∞} \frac{{e}^{i ( 1 + {n}_{1} ) \frac{π}{2}} [ 1 - {e}^{i {n}_{1} ξ} ]}{{n}_{1}} {J}_{{n}_{1}} ( {k}_{0} \vert z - r \vert ) {e}^{- i {n}_{1} {φ}^{′}} + {\sum }_{{n}_{1} = - ∞}^{- 1} \frac{{e}^{i ( 1 + {n}_{1} ) \frac{π}{2}} [ 1 - {e}^{i {n}_{1} ξ} ]}{{n}_{1}} {J}_{{n}_{1}} ( {k}_{0} \vert z - r \vert ) {e}^{- i {n}_{1} {φ}^{′}} = {\sum }_{{n}_{1} = 1}^{∞} \frac{{e}^{i ( 1 + {n}_{1} ) \frac{π}{2}} [ 1 - {e}^{i {n}_{1} ξ} ]}{{n}_{1}} {J}_{{n}_{1}} ( {k}_{0} \vert z - r \vert ) {e}^{- i {n}_{1} {φ}^{′}} + {\sum }_{{n}_{1} = 1}^{∞} \frac{{e}^{i ( 1 + {n}_{1} ) \frac{π}{2}} [ {e}^{- i {n}_{1} ξ} - 1 ]}{{n}_{1}} {J}_{- {n}_{1}} ( {k}_{0} \vert z - r \vert ) {e}^{i {n}_{1} {φ}^{′}} = *$$

Using J₋ₙ_{₁}(k₀|z−r|)=(−1)ⁿ^{₁}Jₙ_{₁}(k₀|z−r|)

$$*= {\sum }_{{n}_{1} = 1}^{∞} \frac{{e}^{i ( 1 + {n}_{1} ) \frac{π}{2}} [ 1 - {e}^{i {n}_{1} ξ} ]}{{n}_{1}} {J}_{{n}_{1}} ( {k}_{0} \vert z - r \vert ) {e}^{- i {n}_{1} {φ}^{′}} + {{\sum }_{{n}_{1} = 1}}^{∞} \frac{{e}^{i ( 1 - {n}_{1} ) \frac{π}{2}} [ {e}^{- i {n}_{1} ξ} - 1 ] {( - 1 )}^{{n}_{1}}}{{n}_{1}} {J}_{{n}_{1}} ( {k}_{0} \vert z - r \vert ) {e}^{i {n}_{1} {φ}^{′}}$$

$${L}_{2} = N ξ {J}_{0} ( {k}_{0} \vert z - r \vert ) {\sum }_{{n}_{1} = 1}^{∞} \frac{{e}^{i \frac{π}{2}}}{{n}_{1}} {J}_{{n}_{1}} ( {k}_{0} \vert z - r \vert ) \{ {e}^{i {n}_{1} \frac{π}{2}} [ 1 - {e}^{i {n}_{1} ξ} ] {e}^{- i {n}_{1} {φ}^{′}} + {e}^{- i {n}_{1} \frac{π}{2}} [ {e}^{- i {n}_{1} ξ} - 1 ] {( - 1 )}^{{n}_{1}} {e}^{i {n}_{1} {φ}^{′}} \}$$

Which is the complex conjugate of L₁ as it should be.

$${L}_{1} = N ξ {J}_{0} ( {k}_{0} \vert z - r \vert ) {\sum }_{{n}_{2} = 1}^{∞} \frac{{e}^{- i \frac{π}{2}}}{{n}_{2}} {J}_{{n}_{2}} ( {k}_{0} \vert z - r \vert ) \{ {e}^{- i {n}_{2} \frac{π}{2}} [ 1 - {e}^{- i {n}_{2} ξ} ] {e}^{i {n}_{2} {φ}^{′}} + {e}^{i {n}_{2} \frac{π}{2}} [ {e}^{i {n}_{2} ξ} - 1 ] {( - 1 )}^{{n}_{2}} {e}^{- i {n}_{2} {φ}^{′}} \}$$

Summing up the terms for n₁ and n₂, i.e. (L₁+L₂) and denoting the index by n:

$$N ξ {J}_{0} ( {k}_{0} \vert z - r \vert ) \times \{ {\sum }_{n = 1}^{∞} \frac{{e}^{- i \frac{π}{2}}}{n} {J}_{n} ( {k}_{0} \vert z - r \vert ) \{ {e}^{- i n \frac{π}{2}} [ 1 - {e}^{- i n ξ} ] {e}^{i n {φ}^{′}} + {e}^{i n \frac{π}{2}} [ {e}^{i n ξ} - 1 ] {( - 1 )}^{n} {e}^{- i n {φ}^{′}} \} + {\sum }_{n = 1}^{∞} \frac{{e}^{i \frac{π}{2}}}{n} {J}_{n} ( {k}_{0} \vert z - r \vert ) \{ {e}^{i n \frac{π}{2}} [ 1 - {e}^{i n ξ} ] {e}^{- i n {φ}^{′}} + {e}^{- i n \frac{π}{2}} [ {e}^{- i n ξ} - 1 ] {( - 1 )}^{n} {e}^{i n {φ}^{′}} \} \}$$

Rearranging terms multiplying e^{-inφ′} and e^{inφ′} separately we get:

$$= N ξ {J}_{0} ( {k}_{0} \vert z - r \vert ) {\sum }_{n = 1}^{∞} \{ \frac{{e}^{i \frac{π}{2}}}{n} {J}_{n} ( {k}_{0} \vert z - r \vert ) {e}^{i n \frac{π}{2}} [ 1 - {e}^{i n ξ} ] - \frac{{e}^{- i \frac{π}{2}}}{n} {e}^{i n \frac{π}{2}} {J}_{n} ( {k}_{0} \vert z - r \vert ) [ 1 - {e}^{i n ξ} ] {( - 1 )}^{n} \} {e}^{- i n {φ}^{′}} + N ξ {J}_{0} ( {k}_{0} \vert z - r \vert ) {\sum }_{n = 1}^{∞} \{ \frac{{e}^{- i \frac{π}{2}}}{n} {J}_{n} ( {k}_{0} \vert z - r \vert ) {e}^{- i n \frac{π}{2}} [ 1 - {e}^{- i n ξ} ] - \frac{{e}^{i \frac{π}{2}}}{n} {e}^{- i n \frac{π}{2}} {J}_{n} ( {k}_{0} \vert z - r \vert ) [ 1 - {e}^{- i n ξ} ] {( - 1 )}^{n} \} {e}^{i n {φ}^{′}}$$

Now inspect the upper term:

$$\{ \frac{{e}^{i \frac{π}{2}}}{n} {J}_{n} ( {k}_{0} \vert z - r \vert ) {e}^{i n \frac{π}{2}} [ 1 - {e}^{i n ξ} ] - \frac{{e}^{- i \frac{π}{2}}}{n} {e}^{i n \frac{π}{2}} {J}_{n} ( {k}_{0} \vert z - r \vert ) [ 1 - {e}^{i n ξ} ] {( - 1 )}^{n} \} {e}^{- i n {φ}^{′}} = \{ \frac{1}{n} {J}_{n} ( {k}_{0} \vert z - r \vert ) {e}^{i n \frac{π}{2}} [ 1 - {e}^{i n ξ} ] [ {e}^{i \frac{π}{2}} - {{e}^{- i \frac{π}{2}} ( - 1 )}^{n} ] \} {e}^{- i n {φ}^{′}} = \{ \frac{{e}^{i \frac{π}{2}}}{n} {J}_{n} ( {k}_{0} \vert z - r \vert ) {e}^{i n \frac{π}{2}} [ 1 - {e}^{i n ξ} ] [ 1 + {( - 1 )}^{n} ] \} {e}^{- i n {φ}^{′}}$$

And similarly for the lower term:

$$\{ \frac{{e}^{- i \frac{π}{2}}}{n} {J}_{n} ( {k}_{0} \vert z - r \vert ) {e}^{- i n \frac{π}{2}} [ 1 - {e}^{- i n ξ} ] - \frac{{e}^{i \frac{π}{2}}}{n} {e}^{- i n \frac{π}{2}} {J}_{n} ( {k}_{0} \vert z - r \vert ) [ 1 - {e}^{- i n ξ} ] {( - 1 )}^{n} \} {e}^{i n {φ}^{′}} = \{ \frac{{e}^{- i \frac{π}{2}}}{n} {J}_{n} ( {k}_{0} \vert z - r \vert ) {e}^{- i n \frac{π}{2}} [ 1 - {e}^{- i n ξ} ] [ 1 + {( - 1 )}^{n} ] \} {e}^{i n {φ}^{′}}$$

This verifies that by adding L₁ and L₂, only even terms contribute to the summation, i.e.

$${L}_{1} + {L}_{2} = N ξ {J}_{0} ( {k}_{0} \vert z - r \vert ) {\sum }_{n = 1}^{∞} \{ \frac{{e}^{i \frac{π}{2}}}{n} {J}_{n} ( {k}_{0} \vert z - r \vert ) {e}^{i n \frac{π}{2}} [ 1 - {e}^{i n ξ} ] [ 1 + {( - 1 )}^{n} ] \} {e}^{- i n {φ}^{′}} + \{ \frac{{e}^{- i \frac{π}{2}}}{n} {J}_{n} ( {k}_{0} \vert z - r \vert ) {e}^{- i n \frac{π}{2}} [ 1 - {e}^{i n ξ} ] [ 1 + {( - 1 )}^{n} ] \} {e}^{i n {φ}^{′}} == 2 N ξ {J}_{0} ( {k}_{0} \vert z - r \vert ) {\sum }_{n = 1 , even}^{∞} \{ \frac{{e}^{i \frac{π}{2}}}{n} {J}_{n} ( {k}_{0} \vert z - r \vert ) {e}^{i n \frac{π}{2}} [ 1 - {e}^{i n ξ} ] \} {e}^{- i n {φ}^{′}} + \{ \frac{{e}^{- i \frac{π}{2}}}{n} {J}_{n} ( {k}_{0} \vert z - r \vert ) {e}^{- i n \frac{π}{2}} [ 1 - {e}^{i n ξ} ] \} {e}^{i n {φ}^{′}}$$

We now Fourier transform

$$\int {d}^{2} ( z - r ) {e}^{- i k \cdot [ z - r ]} ( {L}_{1} + {L}_{2} ) == \int {d}^{2} ( z - r ) {e}^{- i k \cdot [ z - r ]} \times 2 N ξ {J}_{0} ( {k}_{0} \vert z - r \vert ) {\sum }_{n = 1 , even}^{∞} \{ \frac{{e}^{i \frac{π}{2}}}{n} {J}_{n} ( {k}_{0} \vert z - r \vert ) {e}^{i n \frac{π}{2}} [ 1 - {e}^{i n ξ} ] \} {e}^{- i n {φ}^{′}} + \{ \frac{{e}^{- i \frac{π}{2}}}{n} {J}_{n} ( {k}_{0} \vert z - r \vert ) {e}^{- i n \frac{π}{2}} [ 1 - {e}^{i n ξ} ] \} {e}^{i n {φ}^{′}}$$

$$exp \{ - i k \cdot [ z - r ] \} = exp \{ - i \vert k \vert \vert z - r \vert cos ( α ) \} = {[ {\sum }_{{n}_{3} = - ∞}^{∞} {i}^{{n}_{3}} {J}_{{n}_{3}} ( \vert k \vert \vert z - r \vert ) exp ( i {n}_{3} α ) ]}^{*} == {\sum }_{{n}_{3} = - ∞}^{∞} {i}^{- {n}_{3}} {J}_{n} ( \vert k \vert \vert z - r \vert ) exp ( - i {n}_{3} α ) = {\sum }_{{n}_{3} = - ∞}^{∞} {e}^{i {n}_{3}} {J}_{{n}_{3}} ( \vert k \vert \vert z - r \vert ) exp ( - i {n}_{3} ( {φ}^{′} + \frac{π}{2} ) ) = {J}_{0} ( \vert k \vert \vert z - r \vert ) + {\sum }_{{n}_{3} = 1}^{∞} {e}^{i {n}_{3} φ} {J}_{{n}_{3}} ( \vert k \vert \vert z - r \vert ) exp ( - i {n}_{3} ( {φ}^{′} + \frac{π}{2} ) ) + {\sum }_{{n}_{3} = - ∞}^{- 1} {e}^{i {n}_{3} φ} {J}_{{n}_{3}} ( \vert k \vert \vert z - r \vert ) exp ( - i {n}_{3} ( {φ}^{′} + \frac{π}{2} ) ) = {J}_{0} ( \vert k \vert \vert z - r \vert ) + {\sum }_{{n}_{3} = 1}^{∞} {e}^{i {n}_{3} φ} {J}_{{n}_{3}} ( \vert k \vert \vert z - r \vert ) exp ( - i {n}_{3} ( {φ}^{′} + \frac{π}{2} ) ) + {\sum }_{{n}_{3} = 1}^{∞} {e}^{- i {n}_{3} φ} {J}_{- {n}_{3}} ( \vert k \vert \vert z - r \vert ) exp ( i {n}_{3} ( {φ}^{′} + \frac{π}{2} ) ) = * \\ {J}_{- {n}_{3}} ( \vert k \vert \vert z - r \vert ) = {( - 1 )}^{{n}_{3}} {J}_{{n}_{3}} ( \vert k \vert \vert z - r \vert ) *= {J}_{0} ( \vert k \vert \vert z - r \vert ) + {\sum }_{{n}_{3} = 1}^{∞} {e}^{i {n}_{3} φ} {J}_{{n}_{3}} ( \vert k \vert \vert z - r \vert ) exp ( - i {n}_{3} ( {φ}^{′} + \frac{π}{2} ) ) + {\sum }_{{n}_{3} = 1}^{∞} {e}^{- i {n}_{3} φ} {J}_{{n}_{3}} ( \vert k \vert \vert z - r \vert ) exp ( i {n}_{3} ( {φ}^{′} + \frac{π}{2} ) ) {( - 1 )}^{{n}_{3}} =$$

Denoting F for compact notation:

$$F = {J}_{0} ( \vert k \vert \vert z - r \vert ) + {\sum }_{{n}_{3} = 1}^{∞} \{ {e}^{i {n}_{3} φ} {J}_{{n}_{3}} ( \vert k \vert \vert z - r \vert ) {e}^{- i {n}_{3} \frac{π}{2}} {e}^{- i {n}_{3} {φ}^{′}} + {e}^{- i {n}_{3} φ} {J}_{{n}_{3}} ( \vert k \vert \vert z - r \vert ) {{e}^{i {n}_{3} \frac{π}{2}} ( - 1 )}^{{n}_{3}} {e}^{i {n }_{3} {φ}^{′}} \} \\ ⇒ \\ \int {d}^{2} ( z - r ) {e}^{- i k \cdot [ z - r ]} ( {L}_{1} + {L}_{2} ) = {\int }_{0}^{∞} \vert z - r \vert d \vert z - r \vert {\int }_{0}^{2 π} d {φ}^{′} ( {L}_{1} + {L}_{2} ) F = {\int }_{0}^{∞} \vert z - r \vert d \vert z - r \vert {\int }_{0}^{2 π} d {φ}^{′} \times 2 N ξ {J}_{0} ( {k}_{0} \vert z - r \vert ) {\sum }_{n = 1 , even}^{∞} \{ \frac{{e}^{i \frac{π}{2}}}{n} {J}_{n} ( {k}_{0} \vert z - r \vert ) {e}^{i n \frac{π}{2}} [ 1 - {e}^{i n ξ} ] \} {e}^{- i n {φ}^{′}} + \{ \frac{{e}^{- i \frac{π}{2}}}{n} {J}_{n} ( {k}_{0} \vert z - r \vert ) {e}^{- i n \frac{π}{2}} [ 1 - {e}^{- i n ξ} ] \} {e}^{i n {φ}^{′}} \times \{ {J}_{0} ( \vert k \vert \vert z - r \vert ) + {\sum }_{{n}_{3} = 1}^{∞} [ {e}^{i {n}_{3} φ} {J}_{{n}_{3}} ( \vert k \vert \vert z - r \vert ) {e}^{- i {n}_{3} \frac{π}{2}} {e}^{- i {n}_{3} {φ}^{′}} + {e}^{- i {n}_{3} φ} {J}_{{n}_{3}} ( \vert k \vert \vert z - r \vert ) {{e}^{i {n}_{3} \frac{π}{2}} ( - 1 )}^{{n}_{3}} {e}^{i {n }_{3} {φ}^{′}} ] \}$$

Note that:

∫₀^{2π}dφ′e^{inφ′}=∫₀^{2π}dφ′e^{-inφ′}=0

∫₀^{2π}dφ′e^{inφ′}eⁱⁿ^{₃}^{φ′}=∫₀^{2π}dφ′e^{-inφ′}e⁻ⁱⁿ^{₃}^{φ′}=0

∫₀^{2π}dφ′e^{inφ′}e⁻ⁱⁿ^{₃}^{φ′}=∫₀^{2π}dφ′e^{-inφ′}eⁱⁿ^{₃}^{φ′}=2πδ_{n,n}_{₃}

Therefore the only surviving terms are:

$${\int }_{0}^{∞} \vert z - r \vert d \vert z - r \vert {\int }_{0}^{2 π} d {φ}^{′} ( {L}_{1} + {L}_{2} ) F = {\int }_{0}^{∞} \vert z - r \vert d \vert z - r \vert \times 2 N ξ {J}_{0} ( {k}_{0} \vert z - r \vert ) \times \{ {\sum }_{n = 1 , even}^{∞} \{ \frac{{e}^{- i \frac{π}{2}}}{n} {J}_{n} ( {k}_{0} \vert z - r \vert ) {e}^{- i n \frac{π}{2}} [ 1 - {e}^{- i n ξ} ] \} \{ {\sum }_{{n}_{3} = 1}^{∞} [ {e}^{i {n}_{3} φ} {J}_{{n}_{3}} ( \vert k \vert \vert z - r \vert ) {e}^{- i {n}_{3} \frac{π}{2}} ] \} 2 π {δ}_{n , {n}_{3}} + {\sum }_{n = 1 , even}^{∞} \{ \frac{{e}^{i \frac{π}{2}}}{n} {J}_{n} ( {k}_{0} \vert z - r \vert ) {e}^{i n \frac{π}{2}} [ 1 - {e}^{i n ξ} ] \} \{ {\sum }_{{n}_{3} = 1}^{∞} [ {e}^{- i {n}_{3} φ} {J}_{{n}_{3}} ( \vert k \vert \vert z - r \vert ) {{e}^{i {n}_{3} \frac{π}{2}} ( - 1 )}^{{n}_{3}} ] \} 2 π {δ}_{n , {n}_{3}} \} = 4 π N ξ {\int }_{0}^{∞} \vert z - r \vert d \vert z - r \vert {J}_{0} ( {k}_{0} \vert z - r \vert ) {\sum }_{n = 1 , even}^{∞} \{ \frac{{e}^{- i \frac{π}{2}}}{n} {J}_{n} ( {k}_{0} \vert z - r \vert ) {e}^{- i n \frac{π}{2}} [ 1 - {e}^{- i n ξ} ] [ {e}^{i n φ} {J}_{n} ( \vert k \vert \vert z - r \vert ) {e}^{- i n \frac{π}{2}} ] + \frac{{e}^{i \frac{π}{2}}}{n} {J}_{n} ( {k}_{0} \vert z - r \vert ) {e}^{i n \frac{π}{2}} [ 1 - {e}^{i n ξ} ] [ {e}^{- i n φ} {J}_{n} ( \vert k \vert \vert z - r \vert ) {e}^{i n \frac{π}{2}} ] \}$$

We note that the above two terms are complex conjugates of each other, so according to footnote¹²

$$\int {d}^{2} ( z - r ) ( {L}_{1} + {L}_{2} ) F = 4 π N ξ {\int }_{0}^{∞} \vert z - r \vert d \vert z - r \vert {J}_{0} ( {k}_{0} \vert z - r \vert ) \times {\sum }_{n = 1 , even}^{∞} 2 Re \{ \frac{{e}^{- i \frac{π}{2}}}{n} {J}_{n} ( {k}_{0} \vert z - r \vert ) {e}^{- i n \frac{π}{2}} [ 1 - {e}^{- i n ξ} ] [ {e}^{i n φ} {J}_{n} ( \vert k \vert \vert z - r \vert ) {e}^{- i n \frac{π}{2}} ] \} = 4 π N ξ {\sum }_{n = 1 , even}^{∞} 2 Re \{ \frac{{e}^{- i \frac{π}{2}}}{n} {e}^{- i n \frac{π}{2}} [ 1 - {e}^{- i n ξ} ] {e}^{i n φ} {e}^{- i n \frac{π}{2}} \} {I}_{0 , n , n} = *$$

¹² C=A+iB; C+C*=2A=2Re(C)

Inspect:

$$\begin{matrix}\frac{{e}^{- i \frac{π}{2}}}{n} {e}^{- i n \frac{π}{2}} [ 1 - {e}^{- i n ξ} ] {e}^{i n φ} {e}^{- i n \frac{π}{2}} = \frac{{e}^{- i \frac{π}{2}}}{n} {e}^{- i n π} [ 1 - {e}^{- i n ξ} ] {e}^{i n φ} \\ = \frac{{e}^{- i \frac{π}{2}}}{n} {e}^{- i n π} {e}^{i n φ} - \frac{{e}^{- i \frac{π}{2}}}{n} {e}^{- i n ( π + ξ )} {e}^{i n φ} \\ = \frac{{e}^{- i \frac{π}{2}}}{n} [ {e}^{i ( n φ - n π )} - {e}^{i ( n φ - n π - n ξ )} ] \\ = \frac{{e}^{- i \frac{π}{2}}}{n} \\ [ \begin{matrix}cos ( n φ - n π ) + i sin ( n φ - n π ) - \\ cos ( n φ - n π - n ξ ) - i sin ( n φ - n π - n ξ )\end{matrix} ]\end{matrix}$$

Taking the Real part (note that

$${e}^{- i \frac{π}{2}} = cos ( - \frac{π}{2} ) + i sin ( - \frac{π}{2} ) = - i ) \text{:} Re \{ \frac{{e}^{- i \frac{π}{2}}}{n} [ cos ( n φ - n π ) + i sin ( n φ - n π ) - cos ( n φ - n π - n ξ ) - i sin ( n φ - n π - n ξ ) ] \} = \frac{1}{n} Re \{ - i cos ( n φ - n π ) + sin ( n φ - n π ) + icos ( n φ - n π - n ξ ) - sin ( n φ - n π - n ξ ) \} = \frac{1}{n} \{ sin ( n φ - n π ) - sin ( n φ - n π - n ξ ) \}$$

We finally arrive at:

$$\int {d}^{2} ( z - r ) {e}^{- i k \cdot [ z - r ]} \{ {L}_{1} + {L}_{2} \} = 8 π N ξ {\sum }_{n = 1 , even}^{∞} \frac{1}{n} [ sin ( n φ - n π ) - sin ( n φ - n π - n ξ ) ] {I}_{0 , n , n}$$

Using the identity in footnote¹³

$$\int {d}^{2} ( z - r ) {e}^{- i k \cdot [ z - r ]} \{ {L}_{1} + {L}_{2} \} = 8 π N ξ {{\sum }_{n = 1 , even}}^{∞} \frac{1}{n} [ sin ( n φ ) - sin ( n φ - n ξ ) ] {I}_{0 , n , n}$$

For ξ=π we get:

$$sin ( n ( π - φ ) ) = - sin ( n φ - n π )$$

$$sin ( n φ - n π ) = sin ( n φ ) ; n = even \\ ⇒$$

http://en.wikipedia.org/wiki/Angle_addition_formula#Angle_sum_and_difference_identities

sin(a−b)=sin a cos b−cos a sin b

[symbol] sin(a−nπ)=sin a cos nπ−cos a sin nπ=sin a; for n=even

$$\begin{matrix}\int {d}^{2} ( z - r ) {e}^{- i k \cdot [ z - r ]} \{ {L}_{1} + {L}_{2} \} {\vert}_{ξ = π} = 8 π N ξ {\sum }_{{n = 1}_{even}}^{∞} \frac{1}{n} [ sin ( n φ ) - sin ( n φ ) ] {I}_{0 , n , n} \\ = 0\end{matrix}$$

We now look at: ∫d²(z−r)e^{-ik·[z-r]}{L₀}

$$\int {d}^{2} ( z - r ) {e}^{- i k \cdot [ z - r ]} \{ {L}_{0} \} = {\int }_{0}^{∞} \vert z - r \vert d \vert z - r \vert {\int }_{0}^{2 π} d {φ}^{′} {L}_{0} F$$

Where as before

$$F = {J}_{0} ( \vert k \vert \vert z - r \vert ) + {\sum }_{{n}_{3} = 1}^{∞} \{ {e}^{i {n}_{3} φ} {J}_{{n}_{3}} ( \vert k \vert \vert z - r \vert ) {e}^{- i {n}_{3} \frac{π}{2}} {e}^{- i {n}_{3} {φ}^{′}} + {e}^{- i {n}_{3} φ} {J}_{{n}_{3}} ( \vert k \vert \vert z - r \vert ) {{e}^{i {n}_{3} \frac{π}{2}} ( - 1 )}^{{n}_{3}} {e}^{i {n}_{3} {φ}^{′}} \}$$

and L₀=N{ξ²J₀(k₀|z−r|)J₀(k₀|z−r|). We get

$${\int }_{0}^{∞} \vert z - r \vert d \vert z - r \vert {\int }_{0}^{2 π} d {φ}^{′} {L}_{0} F = {\int }_{0}^{∞} \vert z - r \vert d \vert z - r \vert {\int }_{0}^{2 π} d {φ}^{′} N \{ {ξ}^{2} {J}_{0} ( {k}_{0} \vert z - r \vert ) {J}_{0} ( {k}_{0} \vert z - r \vert ) {J}_{0} ( \vert k \vert \vert z - r \vert ) + {\int }_{0}^{∞} d \vert z - r \vert {\int }_{0}^{2 π} d {φ}^{′} N \{ {ξ}^{2} {J}_{0} ( {k}_{0} \vert z - r \vert ) {J}_{0} ( {k}_{0} \vert z - r \vert ) \times {\sum }_{{n}_{3} = 1}^{∞} \{ {e}^{i {n}_{3} φ} {J}_{{n}_{3}} ( \vert k \vert \vert z - r \vert ) {e}^{- i {n}_{3} \frac{π}{2}} {e}^{- i {n}_{3} {φ}^{′}} + {e}^{- i {n}_{3} φ} {J}_{{n}_{3}} ( \vert k \vert \vert z - r \vert ) {{e}^{i {n}_{3} \frac{π}{2}} ( - 1 )}^{{n}_{3}} {e}^{i {n}_{3} {φ}^{′}} \} == 2 π N {ξ}^{2} {I}_{0 , 0 , 0} + {\int }_{0}^{∞} \vert z - r \vert d \vert z - r \vert {\int }_{0}^{2 π} d {φ}^{′} N \{ {ξ}^{2} {J}_{0} ( {k}_{0} \vert z - r \vert ) {J}_{0} ( {k}_{0} \vert z - r \vert ) \times {\sum }_{{n}_{3} = 1}^{∞} \{ {e}^{i {n}_{3} φ} {J}_{{n}_{3}} ( \vert k \vert \vert z - r \vert ) {e}^{- i {n}_{3} \frac{π}{2}} {e}^{- i {n}_{3} {φ}^{′}} + {e}^{- i {n}_{3} φ} {J}_{{n}_{3}} ( \vert k \vert \vert z - r \vert ) {{e}^{i {n}_{3} \frac{π}{2}} ( - 1 )}^{{n}_{3}} {e}^{i {n}_{3} {φ}^{′}} \} {\int }_{0}^{∞} \vert z - r \vert d \vert z - r \vert {\int }_{0}^{2 π} d {φ}^{′} {L}_{0} F = 2 π N {ξ}^{2} {I}_{0 , 0 , 0} ; as {\int }_{0}^{2 π} d {φ}^{′} {e}^{i {n}_{3} {φ}^{′} = 0}$$

Finally we look at:

$$\int {d}^{2} ( z - r ) {e}^{- i k \cdot [ z - r ]} \{ {L}_{3} \}$$

$$\int {d}^{2} ( z - r ) {e}^{- i k \cdot [ z - r ]} \{ {L}_{3} \} = {\int }_{0}^{∞} \vert z - r \vert d \vert z - r \vert {\int }_{0}^{2 π} d {φ}^{′} {L}_{3} F$$

$$F = {J}_{0} ( \vert k \vert \vert z - r \vert ) + {\sum }_{{n}_{3} = 1}^{∞} \{ {e}^{i {n}_{3} φ} {J}_{{n}_{3}} ( \vert k \vert \vert z - r \vert ) {e}^{- i {n}_{3} \frac{π}{2}} {e}^{- i {n}_{3} {φ}^{′}} + {e}^{- i {n}_{3} φ} {J}_{{n}_{3}} ( \vert k \vert \vert z - r \vert ) {{e}^{i {n}_{3} \frac{π}{2}} ( - 1 )}^{{n}_{3}} {e}^{i {n}_{3} {φ}^{′}} \}$$

$${L}_{3} = N [ {\sum }_{{n}_{2} = - ∞ , {n}_{2} \neq 0}^{∞} \frac{{e}^{- i ( 1 + {n}_{2} ) \frac{π}{2}} [ 1 - {e}^{- i {n}_{2} ξ} ]}{{n}_{2}} {J}_{{n}_{2}} ( {k}_{0} \vert z - r \vert ) {e}^{- i {n}_{2} {φ}^{′}} ] \times [ {\sum }_{{n}_{1} = - ∞ , {n}_{1} \neq 0}^{∞} \frac{{e}^{i ( 1 + {n}_{1} ) \frac{π}{2}} [ 1 - {e}^{i {n}_{1} ξ} ]}{{n}_{1}} {J}_{{n}_{1}} ( {k}_{0} \vert z - r \vert ) {e}^{- i {n}_{1} {φ}^{′}} ]$$

As before, we now partition the summation into two parts:

$${L}_{3} = N \times [ {\sum }_{{n}_{2} = 1}^{∞} \frac{{e}^{- i ( 1 + {n}_{2} ) \frac{π}{2}} [ 1 - {e}^{- i {n}_{2} ξ} ]}{{n}_{2}} {J}_{{n}_{2}} ( {k}_{0} \vert z - r \vert ) {e}^{i {n}_{2} {φ}^{′}} + {\sum }_{{n}_{2} = - ∞}^{- 1} \frac{{e}^{- i ( 1 + {n}_{2} ) \frac{π}{2}} [ 1 - {e}^{- i {n}_{2} ξ} ]}{{n}_{2}} {J}_{{n}_{2}} ( {k}_{0} \vert z - r \vert ) {e}^{i {n}_{2} {φ}^{′}} ] \times [ {\sum }_{{n}_{1} = 1}^{∞} \frac{{e}^{i ( 1 + {n}_{1} ) \frac{π}{2}} [ 1 - {e}^{i {n}_{1} ξ} ]}{{n}_{1}} {J}_{{n}_{1}} ( {k}_{0} \vert z - r \vert ) {e}^{- i {n}_{1} {φ}^{′}} + {\sum }_{{n}_{1} = - ∞}^{- 1} \frac{{e}^{i ( 1 + {n}_{1} ) \frac{π}{2}} [ 1 - {e}^{i {n}_{1} ξ} ]}{{n}_{1}} {J}_{{n}_{1}} ( {k}_{0} \vert z - r \vert ) {e}^{- i {n}_{1} {φ}^{′}} ]$$

Again using:

J₋ₙ(|k∥z−r|)=(−1)ⁿJₙ(|k∥z−r|)

We get:

$${L}_{3} = N \times [ {\sum }_{{n}_{2} = 1}^{∞} \frac{{e}^{- i ( 1 + {n}_{2} ) \frac{π}{2}} [ 1 - {e}^{- i {n}_{2} ξ} ]}{{n}_{2}} {J}_{{n}_{2}} ( {k}_{0} \vert z - r \vert ) {e}^{i {n}_{2} {φ}^{′}} + {\sum }_{{n}_{2} = 1}^{∞} \frac{{e}^{- i ( 1 - {n}_{2} ) \frac{π}{2}} [ 1 - {e}^{i {n}_{2} ξ} ]}{- {n}_{2}} {J}_{{n}_{2}} ( {k}_{0} \vert z - r \vert ) {( - 1 )}^{{n}_{2}} {e}^{- i {n}_{2} {φ}^{′}} ] \times [ {\sum }_{{n}_{1} = 1}^{∞} \frac{{e}^{i ( 1 + {n}_{1} ) \frac{π}{2}} [ 1 - {e}^{i {n}_{1} ξ} ]}{{n}_{1}} {J}_{{n}_{1}} ( {k}_{0} \vert z - r \vert ) {e}^{- i {n}_{1} {φ}^{′}} + {\sum }_{{n}_{1} = 1}^{∞} \frac{{e}^{i ( 1 - {n}_{1} ) \frac{π}{2}} [ 1 - {e}^{- i {n}_{1} ξ} ]}{- {n}_{1}} {J}_{{n}_{1}} ( {k}_{0} \vert z - r \vert ) {( - 1 )}^{{n}_{1}} {e}^{i {n}_{1} {φ}^{′}} ]$$

We first look at:

$${\int }_{0}^{∞} \vert z - r \vert d \vert z - r \vert {\int }_{0}^{2 π} d {φ}^{′} {L}_{3} {J}_{0} ( \vert k \vert \vert z - r \vert )$$

Recalling that:

∫₀^{2π}dφ′e^{inφ′}=∫₀^{2π}dφ′e^{-inφ′}=0

∫₀^{2π}dφ′eⁱⁿ^{₁}^{φ′}eⁱⁿ^{₂}^{φ′}=∫₀^{2π}dφ′e⁻ⁱⁿ^{₁}^{φ′}e⁻ⁱⁿ^{₂}^{φ′}=0

∫₀^{2π}dφ′eⁱⁿ^{₁}^{φ′}e⁻ⁱⁿ^{₂}^{φ′}=∫₀^{2π}dφ′e⁻ⁱⁿ^{₁}^{φ′}eⁱⁿ^{₂}^{φ′}=2πδₙ_{₁}_{,n}_{₂}

The only terms remaining are:

$${\int }_{0}^{∞} \vert z - r \vert d \vert z - r \vert {\int }_{0}^{2 π} d {φ}^{′} {L}_{3} {J}_{0} ( \vert k \vert \vert z - r \vert ) = {\int }_{0}^{∞} \vert z - r \vert d \vert z - r \vert {\int }_{0}^{2 π} d {φ}^{′} {J}_{0} ( \vert k \vert \vert z - r \vert ) N \times {\sum }_{{n}_{1} , {n}_{2} = 1}^{∞} \frac{{e}^{- i ( 1 + {n}_{2} ) \frac{π}{2}} [ 1 - {e}^{- i {n}_{2} ξ} ]}{{n}_{2}} {J}_{{n}_{2}} ( {k}_{0} \vert z - r \vert ) {e}^{i {n}_{2} {φ}^{′}} \times \frac{{e}^{i ( 1 + {n}_{1} ) \frac{π}{2}} [ 1 - {e}^{i {n}_{1} ξ} ]}{{n}_{1}} {J}_{{n}_{1}} ( {k}_{0} \vert z - r \vert ) {e}^{- i {n}_{1} {φ}^{′}} + {\sum }_{{n}_{1} , {n}_{2} = 1}^{∞} \frac{{e}^{- i ( 1 - {n}_{2} ) \frac{π}{2}} [ 1 - {e}^{i {n}_{2} ξ} ]}{- {n}_{2}} {J}_{{n}_{2}} ( {k}_{0} \vert z - r \vert ) {( - 1 )}^{{n}_{2}} {e}^{- i {n}_{2} {φ}^{′}} \times \frac{{e}^{i ( 1 - {n}_{1} ) \frac{π}{2}} [ 1 - {e}^{- i {n}_{1} ξ} ]}{- {n}_{1}} {J}_{{n}_{1}} ( {k}_{0} \vert z - r \vert ) {( - 1 )}^{{n}_{1}} {e}^{i {n}_{1} {φ}^{′}} = 2 π N {\int }_{0}^{∞} \vert z - r \vert d \vert z - r \vert {J}_{0} ( \vert k \vert \vert z - r \vert ) \times {\sum }_{n = 1}^{∞} \frac{{e}^{- i ( 1 + n ) \frac{π}{2}} [ 1 - {e}^{- i n ξ} ]}{n} {J}_{n} ( {k}_{0} \vert z - r \vert ) \times \frac{{e}^{i ( 1 + n ) \frac{π}{2}} [ 1 - {e}^{i n ξ} ]}{n} {J}_{n} ( {k}_{0} \vert z - r \vert ) + {\sum }_{n = 1}^{∞} \frac{{e}^{- i ( 1 - n ) \frac{π}{2}} [ 1 - {e}^{i n ξ} ]}{- n} {J}_{n} ( {k}_{0} \vert z - r \vert ) {( - 1 )}^{n} \times \frac{{e}^{i ( 1 - n ) \frac{π}{2}} [ 1 - {e}^{- i n ξ} ]}{- n} {J}_{n} ( {k}_{0} \vert z - r \vert ) {( - 1 )}^{n}$$

$${I}_{{n}_{1} , {n}_{2} , {n}_{3}} = {I}_{{n}_{1} , {n}_{2} {n}_{3}} ( {k}_{0} , \vert k \vert ) = {\int }_{0}^{∞} \vert z - r \vert d \vert z - r \vert {J}_{{n}_{1}} ( {k}_{0} \vert z - r \vert ) {J}_{{n}_{2}} ( {k}_{0} \vert z - r \vert ) {J}_{{n}_{3}} ( \vert k \vert \vert z - r \vert ) = 2 π N {\sum }_{n = 1}^{∞} [ \frac{{e}^{- i ( 1 + n ) \frac{π}{2}} [ 1 - {e}^{- i n ξ} ]}{n} \frac{{e}^{i ( 1 + n ) \frac{π}{2}} [ 1 - {e}^{i n ξ} ]}{n} + \frac{{e}^{- i ( 1 - n ) \frac{π}{2}} [ 1 - {e}^{i n ξ} ]}{- n} \frac{{e}^{i ( 1 - n ) \frac{π}{2}} [ 1 - {e}^{- i n ξ} ]}{- n} {I}_{n , n , 0} ] = 2 π N {\sum }_{n = 1}^{∞} [ \frac{[ 1 - {e}^{- i n ξ} ]}{n} \frac{[ 1 - {e}^{i n ξ} ]}{n} + \frac{[ 1 - {e}^{i n ξ} ]}{- n} \frac{[ 1 - {e}^{- i n ξ} ]}{- n} {I}_{n , n , 0} ] = 2 π N {\sum }_{n = 1}^{∞} \frac{2}{{n}^{2}} [ 1 - {e}^{- i n ξ} ] [ 1 - {e}^{i n ξ} ] {I}_{0 , n , n} = [ 1 - {e}^{- i n ξ} ] [ 1 - {e}^{i n ξ} ] = 2 - {e}^{- i n ξ} - {e}^{i n ξ} = 2 - cos ( - n ξ ) - i sin ( - n ξ ) - cos ( n ξ ) - isin ( n ξ ) = - 2 [ 1 - cos ( n ξ ) ]$$

$${\int }_{0}^{∞} \vert z - r \vert d \vert z - r \vert {\int }_{0}^{2 π} d {φ}^{′} {L}_{3} {J}_{0} ( \vert k \vert \vert z - r \vert ) = 8 π N {\sum }_{n = 1}^{∞} \frac{1}{{n}^{2}} [ 1 - cos ( n ξ ) ] {I}_{n , n , 0}$$

For the case ξ=π we get

$$8 π N {\sum }_{n = 1}^{∞} \frac{1}{{n}^{2}} [ 1 - {( - 1 )}^{n} ] {I}_{0 , n , n} = 16 π N {\sum }_{n = 1 , odd}^{∞} \frac{1}{{n}^{2}} {I}_{n , n , 0}$$

We secondly look at the contribution of the second term of F:

$${\int }_{0}^{∞} \vert z - r \vert d \vert z - r \vert {\int }_{0}^{2 π} d {φ}^{′} {L}_{3} {\sum }_{{n}_{3} = 1}^{∞} \{ {e}^{i {n}_{3} φ} {J}_{{n}_{3}} ( \vert k \vert \vert z - r \vert ) {e}^{- i {n}_{3} \frac{π}{2}} {e}^{- i {n}_{3} {φ}^{′}} + {e}^{- i {n}_{3} φ} {J}_{{n}_{3}} ( \vert k \vert \vert z - r \vert ) {{e}^{i {n}_{3} \frac{π}{2}} ( - 1 )}^{{n}_{3}} {e}^{i {n}_{3} {φ}^{′}} \}$$

$$E = {\int }_{0}^{∞} \vert z - r \vert d \vert z - r \vert {\int }_{0}^{2 π} d {φ}^{′} N \times [ {\sum }_{{n}_{3} = - ∞ , {n}_{3} \neq 0}^{∞} {e}^{i {n}_{3} φ} {J}_{{n}_{3}} ( \vert k \vert \vert z - r \vert ) {e}^{- i {n}_{3} \frac{π}{2}} {e}^{- i {n}_{3} {φ}^{′}} ] [ {\sum }_{{n}_{2} = - ∞ , {n}_{2} \neq 0}^{∞} \frac{{e}^{- i ( 1 + {n}_{2} ) \frac{π}{2}} [ 1 - {e}^{- i {n}_{2} ξ} ]}{{n}_{2}} {J}_{{n}_{2}} ( {k}_{0} \vert z - r \vert ) {e}^{i {n}_{2} {φ}^{′}} ] [ {\sum }_{{n}_{1} = - ∞ , {n}_{1} \neq 0}^{∞} \frac{{e}^{i ( 1 + {n}_{1} ) \frac{π}{2}} [ 1 - {e}^{i {n}_{1} ξ} ]}{{n}_{1}} {J}_{{n}_{1}} ( {k}_{0} \vert z - r \vert ) {e}^{- i {n}_{1} {φ}^{′}} ] \\ E = {\int }_{0}^{∞} \vert z - r \vert d \vert z - r \vert {\int }_{0}^{2 π} d {φ}^{′} N \times {e}^{i ( {n}_{1} - {n}_{2} - {n}_{3} ) \frac{π}{2}} \frac{[ 1 - {e}^{i {n}_{1} ξ} ]}{{n}_{1}} \frac{[ 1 - {e}^{- i {n}_{2} ξ} ]}{{n}_{2}} {e}^{i {n}_{3} φ} {\sum }_{{n}_{1} , {n}_{2} , {n}_{3} = - ∞ , {n}_{1} , {n}_{2} , {n}_{3} \neq 0}^{∞} {J}_{{n}_{1}} ( {k}_{0} \vert z - r \vert ) {J}_{{n}_{2}} ( {k}_{0} \vert z - r \vert ) {J}_{{n}_{3}} ( \vert k \vert \vert z - r \vert ) {e}^{- i {n}_{1} {φ}^{′}} {e}^{i {n}_{2} {φ}^{′}} {e}^{- i {n}_{3} {φ}^{′}}$$

We define and note:

$$E = {\int }_{0}^{∞} \vert z - r \vert d \vert z - r \vert {\int }_{0}^{2 π} d {φ}^{′} N \times {e}^{i ( {n}_{1} - {n}_{2} - {n}_{3} ) \frac{π}{2}} \frac{[ 1 - {e}^{i {n}_{1} ξ} ]}{{n}_{1}} \frac{[ 1 - {e}^{- i {n}_{2} ξ} ]}{{n}_{2}} {e}^{i {n}_{3} φ} {{\sum }_{{n}_{1} , {n}_{2} , {n}_{3} = - ∞ , {n}_{1} , {n}_{2} , {n}_{3} \neq 0}}^{∞} {J}_{{n}_{1}} ( {k}_{0} \vert z - r \vert ) {J}_{{n}_{2}} ( {k}_{0} \vert z - r \vert ) {J}_{{n}_{3}} ( \vert k \vert \vert z - r \vert ) {e}^{- i {n}_{1} {φ}^{′}} {e}^{i {n}_{2} {φ}^{′}} {e}^{- i {n}_{3} {φ}^{′}}$$

We now partition the summation into sections:

$$E = 2 π N \times \{ \begin{matrix}{\sum }_{{n}_{1} = 1 , {n}_{2} = 1 , {n}_{3} = 1}^{∞ , ∞ , ∞} {M}_{n 1 , {n}_{2} , {n}_{3}} {I}_{{n}_{1} , {n}_{2} , {n}_{3}} {δ}_{- {n}_{1} + {n}_{2} , {n}_{3}} + \\ {\sum }_{{n}_{1} = - ∞ , {n}_{2} = 1 , {n}_{3} = 1}^{- 1 , ∞ , ∞} {M}_{n 1 , {n}_{2} , {n}_{3}} {I}_{{n}_{1} , {n}_{2} , {n}_{3}} {δ}_{- {n}_{1} + {n}_{2} , {n}_{3}} + \\ {\sum }_{{n}_{1} = 1 , {n}_{2} = - ∞ , {n}_{3} = 1}^{∞ , - 1 , ∞} {M}_{n 1 , {n}_{2} , {n}_{3}} {I}_{{n}_{1} , {n}_{2} , {n}_{3}} {δ}_{- {n}_{1} + {n}_{2} , {n}_{3}} + \\ {\sum }_{{n}_{1} = 1 , {n}_{2} = 1 , {n}_{3} = - ∞}^{∞ , ∞ , - 1} {M}_{n 1 , {n}_{2} , {n}_{3}} {I}_{{n}_{1} , {n}_{2} , {n}_{3}} {δ}_{- {n}_{1} + {n}_{2} , {n}_{3}} + \\ {\sum }_{{n}_{1} = - ∞ , {n}_{2} = - ∞ , {n}_{3} = 1}^{- 1 , - 1 , ∞} {M}_{n 1 , {n}_{2} , {n}_{3}} {I}_{{n}_{1} , {n}_{2} , {n}_{3}} {δ}_{- {n}_{1} + {n}_{2} , {n}_{3}} + \\ {\sum }_{{n}_{1} = 1 , {n}_{2} = - ∞ , {n}_{3} = - ∞}^{∞ , - 1 , - 1} {M}_{n 1 , {n}_{2} , {n}_{3}} {I}_{{n}_{1} , {n}_{2} , {n}_{3}} {δ}_{- {n}_{1} + {n}_{2} , {n}_{3}} + \\ {\sum }_{{n}_{1} = - ∞ , {n}_{2} = 1 , {n}_{3} = - ∞}^{- 1 , ∞ , - 1} {M}_{n 1 , {n}_{2} , {n}_{3}} {I}_{{n}_{1} , {n}_{2} , {n}_{3}} {δ}_{- {n}_{1} + {n}_{2} , {n}_{3}} + \\ {\sum }_{{n}_{1} = - ∞ , {n}_{2} = - ∞ , {n}_{3} = - ∞}^{- 1 , - 1 , - 1} {M}_{n 1 , {n}_{2} , {n}_{3}} {I}_{{n}_{1} , {n}_{2} , {n}_{3}} {δ}_{- {n}_{1} + {n}_{2} , {n}_{3}}\end{matrix}$$

Changing the summations into ‘1 to ∞’ and changing the corresponding n index to −n:

$$E = 2 π N \times \{ \begin{matrix}{\sum }_{{n}_{1} , {n}_{2} , {n}_{3} = 1}^{∞} {M}_{n 1 , {n}_{2} , {n}_{3}} {I}_{{n}_{1} , {n}_{2} , {n}_{3}} {δ}_{- {n}_{1} + {n}_{2} , {n}_{3}} + \\ {\sum }_{{n}_{1} , {n}_{2} , {n}_{3} = 1}^{∞} {M}_{- n 1 , {n}_{2} , {n}_{3}} {I}_{- {n}_{1} , {n}_{2} , {n}_{3}} {δ}_{{n}_{1} + {n}_{2} , {n}_{3}} + \\ {\sum }_{{n}_{1} , {n}_{2} , {n}_{3} = 1}^{∞} {M}_{n 1 , {n}_{2} , {n}_{3}} {I}_{{n}_{1} , {n}_{2} , - {n}_{3}} {δ}_{- {n}_{1} + {n}_{2} , - {n}_{3}} + \\ {\sum }_{{n}_{1} , {n}_{2} , {n}_{3} = 1}^{∞} {M}_{- n 1 , - {n}_{2} , {n}_{3}} {I}_{- {n}_{1} , - {n}_{2} , {n}_{3}} {δ}_{{n}_{1} - {n}_{2} , {n}_{3}} + \\ {\sum }_{{n}_{1} , {n}_{2} , {n}_{3} = 1}^{∞} {M}_{n 1 , - {n}_{2} , - {n}_{3}} {I}_{{n}_{1} , - {n}_{2} , - {n}_{3}} {δ}_{- {n}_{1} - {n}_{2} , - {n}_{3}} + \\ {\sum }_{{n}_{1} , {n}_{2} , {n}_{3} = 1}^{∞} {M}_{- n 1 , - {n}_{2} , - {n}_{3}} {I}_{- {n}_{1} , - {n}_{2} , - {n}_{3}} {δ}_{{n}_{1} - {n}_{2} , - {n}_{3}}\end{matrix}$$

Rearranging terms:

$$E = 2 π N \times \{ \begin{matrix}{\sum }_{{n}_{1} , {n}_{2} , {n}_{3} = 1}^{∞} {M}_{n 1 , {n}_{2} , {n}_{3}} {I}_{{n}_{1} , {n}_{2} , {n}_{3}} {δ}_{- {n}_{1} + {n}_{2} , {n}_{3}} + \\ {\sum }_{{n}_{1} , {n}_{2} , {n}_{3} = 1}^{∞} {M}_{- n 1 , - {n}_{2} , - {n}_{3}} {I}_{- {n}_{1} , - {n}_{2} , - {n}_{3}} {δ}_{{n}_{1} - {n}_{2} , - {n}_{3}} + \\ {\sum }_{{n}_{1} , {n}_{2} , {n}_{3} = 1}^{∞} {M}_{- n 1 , - {n}_{2} , {n}_{3}} {I}_{- {n}_{1} , - {n}_{2} , {n}_{3}} {δ}_{{n}_{1} - {n}_{2} , {n}_{3}} + \\ {\sum }_{{n}_{1} , {n}_{2} , {n}_{3} = 1}^{∞} {M}_{n 1 , {n}_{2} , - {n}_{3}} {I}_{{n}_{1} , {n}_{2} , - {n}_{3}} {δ}_{- {n}_{1} + {n}_{2} , - {n}_{3}} + \\ {\sum }_{{n}_{1} , {n}_{2} , {n}_{3} = 1}^{∞} {M}_{- n 1 , {n}_{2} , {n}_{3}} {I}_{- {n}_{1} , {n}_{2} , {n}_{3}} {δ}_{{n}_{1} + {n}_{2} , {n}_{3}} + \\ {\sum }_{{n}_{1} , {n}_{2} , {n}_{3} = 1}^{∞} {M}_{n 1 , - {n}_{2} , - {n}_{3}} {I}_{{n}_{1} , - {n}_{2} , - {n}_{3}} {δ}_{- {n}_{1} - {n}_{2} , - {n}_{3}}\end{matrix}$$

Noting the properties of δ_{n,n′}:

$$E = 2 π N \times \{ \begin{matrix}{\sum }_{{n}_{1} , {n}_{2} , {n}_{3} = 1}^{∞} [ {M}_{n 1 , {n}_{2} , {n}_{3}} {I}_{{n}_{1} , {n}_{2} , {n}_{3}} + {M}_{- n 1 , - {n}_{2} , - {n}_{3}} {I}_{- {n}_{1} , - {n}_{2} , - {n}_{3}} ] {δ}_{- {n}_{1} + {n}_{2} , {n}_{3}} + \\ {\sum }_{{n}_{1} , {n}_{2} , {n}_{3} = 1}^{∞} [ {M}_{- n 1 , - {n}_{2} , {n}_{3}} {I}_{- {n}_{1} , - {n}_{2} , {n}_{3}} + {M}_{n 1 , {n}_{2} , - {n}_{3}} {I}_{{n}_{1} , {n}_{2} , - {n}_{3}} ] {δ}_{{n}_{1} - {n}_{2} , {n}_{3}} + \\ {\sum }_{{n}_{1} , {n}_{2} , {n}_{3} = 1}^{∞} [ {M}_{- n 1 , {n}_{2} , {n}_{3}} {I}_{- {n}_{1} , {n}_{2} , {n}_{3}} + {M}_{n 1 , - {n}_{2} , - {n}_{3}} {I}_{{n}_{1} , - {n}_{2} , - {n}_{3}} ] {δ}_{{n}_{1} + {n}_{2} , {n}_{3}}\end{matrix}$$

Using the δ:

$$E = 2 π N \times \{ \begin{matrix}{\sum }_{{n}_{1} , {n}_{2} , {n}_{3} = 1 - {n}_{1} + {n}_{2} \geq 1}^{∞} [ \begin{matrix}{M}_{{n}_{1} , {n}_{2} , - {n}_{1} + {n}_{2}} {I}_{{n}_{1} , {n}_{2} , - {n}_{1} + {n}_{2}} + \\ {M}_{- {n}_{1} , - {n}_{2} , {n}_{1} - {n}_{2}} {I}_{- {n}_{1} , - {n}_{2} , {n}_{1} - {n}_{2}}\end{matrix} ] + \\ {\sum }_{{n}_{1} , {n}_{2} = 1 {n}_{1} - {n}_{2} \geq 1}^{∞} [ \begin{matrix}{M}_{- {n}_{1} , - {n}_{2} , {n}_{1} - {n}_{2}} {I}_{- {n}_{1} , - {n}_{2} , {n}_{1} - {n}_{2}} + \\ {M}_{{n}_{1} , {n}_{2} , - {n}_{1} + {n}_{2}} {I}_{{n}_{1} , {n}_{2} , - {n}_{1} + {n}_{2}}\end{matrix} ] + \\ {\sum }_{{n}_{1} , {n}_{2} = 1}^{∞} [ \begin{matrix}{M}_{- {n}_{1} , {n}_{2} , {n}_{1} + {n}_{2}} {I}_{- {n}_{1} , {n}_{2} , {n}_{1} + {n}_{2}} + \\ {M}_{{n}_{1} , - {n}_{2} , - {n}_{1} - {n}_{2}} {I}_{{n}_{1} , - {n}_{2} , - {n}_{1} - {n}_{2}}\end{matrix} ]\end{matrix}$$

Collecting terms for −n₁+n₂≧1 and n₁−n₂≧1 we get:

$$E = 2 π N \times \{ \begin{matrix}{\sum }_{{n}_{1} , {n}_{2} = 1 {n}_{2} - {n}_{1} , \neq 0}^{∞} [ \begin{matrix}{M}_{{n}_{1} , {n}_{2} , - {n}_{1} + {n}_{2}} {I}_{{n}_{1} , {n}_{2} , - {n}_{1} + {n}_{2}} + \\ {M}_{- {n}_{1} , - {n}_{2} , {n}_{1} - {n}_{2}} {I}_{- {n}_{1} , - {n}_{2} , {n}_{1} - {n}_{2}}\end{matrix} ] + \\ {\sum }_{{n}_{1} , {n}_{2} = 1}^{∞} [ \begin{matrix}{M}_{- {n}_{1} , {n}_{2} , {n}_{1} + {n}_{2}} {I}_{- {n}_{1} , {n}_{2} , {n}_{1} + {n}_{2}} + \\ {M}_{{n}_{1} , - {n}_{2} , - {n}_{1} - {n}_{2}} {I}_{{n}_{1} , - {n}_{2} , - {n}_{1} - {n}_{2}}\end{matrix} ]\end{matrix}$$

From the explicit expression of Mₙ_{₁}_{,n}_{₂}_{,n}_{₃} we see that:

$$E = 2 π N \times \{ \begin{matrix}{\sum }_{{n}_{1} , {n}_{2} = 1 {n}_{2} - {n}_{1} , \neq 0}^{∞} [ \begin{matrix}{M}_{{n}_{1} , {n}_{2} , - {n}_{1} + {n}_{2}} {I}_{{n}_{1} , {n}_{2} , - {n}_{1} + {n}_{2}} + \\ {M}_{{n}_{1} , {n}_{2} , - {n}_{1} + {n}_{2}}^{*} {I}_{- {n}_{1} , - {n}_{2} , {n}_{1} - {n}_{2}}\end{matrix} ] + \\ {\sum }_{{n}_{1} , {n}_{2} = 1}^{∞} [ \begin{matrix}{M}_{- {n}_{1} , {n}_{2} , {n}_{1} + {n}_{2}} {I}_{- {n}_{1} , {n}_{2} , {n}_{1} + {n}_{2}} + \\ {M}_{- {n}_{1} , {n}_{2} , {n}_{1} + {n}_{2}}^{*} {I}_{{n}_{1} , - {n}_{2} , - {n}_{1} - {n}_{2}}\end{matrix} ]\end{matrix}$$

From the properties Iₙ_{₁}_{,n}_{₂}_{,n}_{₃}:

$${I}_{- {n}_{1} , - {n}_{2} , {n}_{1} - {n}_{2}} = {( - 1 )}^{{n}_{1}} {( - 1 )}^{{n}_{2}} {( - 1 )}^{- {n}_{1} + {n}_{2}} {I}_{{n}_{1} , {n}_{2} , - {n}_{1} + {n}_{2}} = {I}_{{n}_{1} , {n}_{2} - {n}_{1} + {n}_{2}}$$

$${I}_{{n}_{1} , - {n}_{2} , - {n}_{1} - {n}_{2}} = {( - 1 )}^{{n}_{2}} {( - 1 )}^{- {n}_{1} + {n}_{2}} {I}_{{n}_{1} , {n}_{2} , {n}_{1} + {n}_{2}} = {( - 1 )}^{{n}_{1}} {I}_{{n}_{1} , {n}_{2} , {n}_{1} + {n}_{2}} ;$$

$${I}_{- {n}_{1} , {n}_{2} , {n}_{1} + {n}_{2}} = {( - 1 )}^{{n}_{1}} {I}_{{n}_{1} , {n}_{2} , {n}_{1} + {n}_{2}}$$

$$E = 2 π N \times \{ \begin{matrix}{\sum }_{{n}_{1} , {n}_{2} = 1 {n}_{2} - {n}_{1} , \neq 0}^{∞} [ 2 Re {M}_{{n}_{1} , {n}_{2} , - {n}_{1} + {n}_{2}} ] {I}_{{n}_{1} , {n}_{2} , - {n}_{1} + {n}_{2}} + \\ {\sum }_{{n}_{1} , {n}_{2} = 1}^{∞} {( - 1 )}^{{n}_{1}} [ 2 Re {M}_{- {n}_{1} , {n}_{2} , {n}_{1} + {n}_{2}} ] {I}_{{n}_{1} , {n}_{2} , {n}_{1} + {n}_{2}}\end{matrix} \}$$

We now recall the expression for

$${M}_{{n}_{1} , {n}_{2} , {n}_{3}} = {e}^{i ( {n}_{1} - {n}_{2} - {n}_{3} ) \frac{π}{2}} [ 1 - {e}^{i {n}_{1} ξ} - {e}^{- i {n}_{2} ξ} + {e}^{i ( {n}_{1} - {n}_{2} ) ξ} ] {e}^{i {n}_{3} φ}$$

$${M}_{{n}_{1} , {n}_{2} , - {n}_{1} + {n}_{2}} = \frac{{e}^{i ( {n}_{1} - {n}_{2} + {n}_{1} - {n}_{2} ) \frac{π}{2}}}{{n}_{1} {n}_{2}} [ 1 - {e}^{i {n}_{1} ξ} - {e}^{- i {n}_{2} ξ} + {e}^{i ( {n}_{1} - {n}_{2} ) ξ} ] {e}^{i ( - {n}_{1} + {n}_{2} ) φ} \\ {e}^{i n π} = cos ( n π ) + i sin ( n π ) = cos ( n π ) = {( - 1 )}^{n} \\ [ 1 - {e}^{i {n}_{1} ξ} - {e}^{- i {n}_{2} ξ} + {e}^{i ( {n}_{1} - {n}_{2} ) ξ} ] == 1 - cos ( {n}_{1} ξ ) - i sin ( {n}_{1} ξ ) - cos ( {n}_{2} ξ ) + i sin ( {n}_{2} ξ ) + cos ( ( {n}_{1} - {n}_{2} ) ξ ) + i sin ( ( {n}_{1} - {n}_{2} ) ξ ) = 1 - cos ( {n}_{1} ξ ) - cos ( {n}_{2} ξ ) + cos ( ( {n}_{1} - {n}_{2} ) ξ ) - i [ sin ( {n}_{1} ξ ) - sin ( {n}_{2} ξ ) - sin ( ( {n}_{1} - {n}_{2} ) ξ ) ] \\ {e}^{i ( - {n}_{1} + {n}_{2} ) φ} = cos ( ( - {n}_{1} + {n}_{2} ) φ ) + i sin ( ( - {n}_{1} + {n}_{2} ) φ ) \\ {ReM}_{{n}_{1} , {n}_{2} , - {n}_{1} + {n}_{2}} = \frac{{( - 1 )}^{{n}_{1} - {n}_{2}}}{{n}_{1} {n}_{2}} \{ [ 1 - cos ( {n}_{1} ξ ) - cos ( {n}_{2} ξ ) + cos ( ( {n}_{1} - {n}_{2} ) ξ ) ] cos ( ( - {n}_{1} + {n}_{2} ) φ ) + [ sin ( {n}_{1} ξ ) - sin ( {n}_{2} ξ ) - sin ( ( {n}_{1} - {n}_{2} ) ξ ) ] sin ( ( - {n}_{1} + {n}_{2} ) φ ) \}$$

We note that if

$${ReM}_{{n}_{1} , {n}_{2} , - {n}_{1} + {n}_{2}} = {ReM}_{n , n , 0} = \frac{1}{{n}^{2}} \{ [ 1 - cos ( n ξ ) - cos ( n ξ ) + cos ( ( 0 ) ξ ) ] cos ( ( 0 ) φ ) + \text{[} sin ( n ξ ) - sin ( n ξ ) - sin 0 ξ sin 0 φ = 1 n 221 - cos n ξ \\ 2 π N \times {\{ {\sum }_{{n}_{1} , {n}_{2} = 1}^{∞} [ 2 Re {M}_{{n}_{1} , {n}_{2} , - {n}_{1} + {n}_{2}} ] {I}_{{n}_{1} , {n}_{2} , - {n}_{1} + {n}_{2}} \}}_{{n}_{2} - {n}_{1} , = 0} = 8 π n {\sum }_{n = 1}^{∞} \frac{1}{{n}^{2}} [ 1 - cos ( n ξ ) ] {I}_{n , n , 0}$$

Which is exactly the result from the second isotropic term. Therefore, if we allow n₁=n₂ we can ommit the second isotropic term.

Similarly again

$${M}_{{n}_{1} , {n}_{2} , {n}_{3}} = \frac{{e}^{i ( {n}_{1} - {n}_{2} - {n}_{3} ) \frac{π}{2}}}{{n}_{1} {n}_{2}} [ 1 - {e}^{i {n}_{1} ξ} - {e}^{- i {n}_{2} ξ} + {e}^{i ( {n}_{1} - {n}_{2} ) ξ} ] {e}^{i {n}_{3} φ} \\ ⇒ \\ {M}_{- {n}_{1} , {n}_{2} , {n}_{3}} = - \frac{{e}^{i ( - {n}_{1} - {n}_{2} - {n}_{3} ) \frac{π}{2}}}{{n}_{1} {n}_{2}} [ 1 - {e}^{- i {n}_{1} ξ} - {e}^{- i {n}_{2} ξ} + {e}^{i ( - {n}_{1} - {n}_{2} ) ξ} ] {e}^{i {n}_{3} φ}$$

$${M}_{- {n}_{1} , {n}_{2} , {n}_{1} + {n}_{2}} = - \frac{{e}^{i ( - {n}_{1} - {n}_{2} ) π}}{{n}_{1} {n}_{2}} [ 1 - {e}^{- i {n}_{1} ξ} - {e}^{- i {n}_{2} ξ} + {e}^{i ( - {n}_{1} - {n}_{2} ) ξ} ] {e}^{i ( {n}_{1} + {n}_{2} ) φ} \\ [ {M}_{- {n}_{1} , {n}_{2} , {n}_{1} + {n}_{2}} ] = - \frac{{( - 1 )}^{{n}_{1} + {n}_{2}}}{{n}_{1} {n}_{2}} [ 1 - {e}^{- i {n}_{1} ξ} - {e}^{- i {n}_{2} ξ} + {e}^{i ( - {n}_{1} - {n}_{2} ) ξ} ] {e}^{i ( {n}_{1} + {n}_{2} ) φ} \\ [ 1 - {e}^{- i {n}_{1} ξ} - {e}^{- i {n}_{2} ξ} + {e}^{i ( - {n}_{1} - {n}_{2} ) ξ} ] = 1 - cos ( - {n}_{1} ξ ) - i sin ( - {n}_{1} ξ ) - cos ( - {n}_{2} ξ ) - i sin ( - {n}_{2} ξ ) + cos ( - ( {n}_{1} + {n}_{2} ) ξ ) + sin ( - ( {n}_{1} + {n}_{2} ) ξ ) = 1 - cos ( {n}_{1} ξ ) + i sin ( {n}_{1} ξ ) - cos ( {n}_{2} ξ ) + i sin ( {n}_{2} ξ ) + cos ( ( {n}_{1} + {n}_{2} ) ξ ) - i sin ( ( {n}_{1} + {n}_{2} ) ξ ) = [ 1 - cos ( {n}_{1} ξ ) - cos ( {n}_{2} ξ ) + cos ( ( {n}_{1} + {n}_{2} ) ξ ) ] + i [ sin ( {n}_{1} ξ ) + sin ( {n}_{2} ξ ) - sin ( ( {n}_{1} + {n}_{2} ) ξ ) ]$$

$${e}^{i ( {n}_{1} + {n}_{2} ) φ} = cos ( ( {n}_{1} + {n}_{2} ) φ ) + i sin ( ( {n}_{1} + {n}_{2} ) φ ) \\ [ 1 - {e}^{- i {n}_{1} ξ} - {e}^{- i {n}_{2} ξ} + {e}^{i ( - {n}_{1} - {n}_{2} ) ξ} ] {e}^{i ( {n}_{1} + {n}_{2} ) φ} = [ 1 - cos ( {n}_{1} ξ ) - cos ( {n}_{2} ξ ) + cos ( ( {n}_{1} + {n}_{2} ) ξ ) ] cos ( ( {n}_{1} + {n}_{2} ) φ ) - [ sin ( {n}_{1} ξ ) + sin ( {n}_{2} ξ ) - sin ( ( {n}_{1} + {n}_{2} ) ξ ) ] sin ( ( {n}_{1} + {n}_{2} ) φ ) \\ i [ 1 - cos ( {n}_{1} ξ ) - cos ( {n}_{2} ξ ) + cos ( ( {n}_{1} + {n}_{2} ) ξ ) ] sin ( ( {n}_{1} + {n}_{2} ) φ ) + i [ sin ( {n}_{1} ξ ) + sin ( {n}_{2} ξ ) - sin ( ( {n}_{1} + {n}_{2} ) ξ ) ] cos ( ( {n}_{1} + {n}_{2} ) φ ) \\ Re [ {M}_{- {n}_{1} , {n}_{2} , {n}_{1} + {n}_{2}} ] = - \frac{{( - 1 )}^{{n}_{1} + {n}_{2}}}{{n}_{1} {n}_{2}} \{ [ 1 - cos ( {n}_{1} ξ ) - cos ( {n}_{2} ξ ) + cos ( ( {n}_{1} + {n}_{2} ) ξ ) ] cos ( ( {n}_{1} + {n}_{2} ) φ ) + [ - sin ( {n}_{1} ξ ) - sin ( {n}_{2} ξ ) + sin ( ( {n}_{1} + {n}_{2} ) ξ ) ] sin ( ( {n}_{1} + {n}_{2} ) φ ) \} \\ E = 4 π N {\sum }_{{n}_{1} , {n}_{2} = 1}^{∞} \frac{{( - 1 )}^{{n}_{1} - {n}_{2}}}{{n}_{1} {n}_{2}} \{ [ 1 - cos ( {n}_{1} ξ ) - cos ( {n}_{2} ξ ) + cos ( ( {n}_{1} - {n}_{2} ) ξ ) ] cos ( ( - {n}_{1} + {n}_{2} ) φ ) + [ sin ( {n}_{1} ξ ) - sin ( {n}_{2} ξ ) - sin ( ( {n}_{1} - {n}_{2} ) ξ ) ] sin ( ( - {n}_{1} + {n}_{2} ) φ ) \} {I}_{{n}_{1} , {n}_{2} , - {n}_{1} + {n}_{2}} - 4 π N {\sum }_{{n}_{1} , {n}_{2} = 1}^{∞} \frac{{( - 1 )}^{{n}_{2}}}{{n}_{1} {n}_{2}} \{ [ 1 - cos ( {n}_{1} ξ ) - cos ( {n}_{2} ξ ) + cos ( ( {n}_{1} + {n}_{2} ) ξ ) ] cos ( ( {n}_{1} + {n}_{2} ) φ ) + [ - sin ( {n}_{1} ξ ) - sin ( {n}_{2} ξ ) + sin ( ( {n}_{1} + {n}_{2} ) ξ ) ] sin ( ( {n}_{1} + {n}_{2} ) φ ) \} {I}_{{n}_{1} , {n}_{2} , {n}_{1} + {n}_{2}}$$

For the case ξ=π we get

$$[ 1 - cos ( {n}_{1} π ) - cos ( {n}_{2} π ) + cos ( ( {n}_{1} - {n}_{2} ) π ) ] = [ 1 - {( - 1 )}^{{n}_{1}} - {( - 1 )}^{{n}_{2}} + {( - 1 )}^{{n}_{1} - {n}_{2}} ] = \{ \begin{matrix}4 , & ( {n}_{1} , {n}_{2} both odd ) \\ 0 , & \begin{matrix}( {n}_{1} , {n}_{2} both even ) or \\ ( {n}_{1} odd and {n}_{2} even ) or \\ ( {n}_{1} even and {n}_{2} odd )\end{matrix}\end{matrix} \\ [ sin ( {n}_{1} π ) - i sin ( {n}_{2} π ) - i sin ( ( {n}_{1} - {n}_{2} ) π ) ] = 0 ; \\ [ - sin ( {n}_{1} π ) - sin ( {n}_{2} π ) + sin ( ( {n}_{1} + {n}_{2} ) π ) ] = 0$$

We note that for odd n₁,n₂ we get (−1)ⁿ^{₂}=−1 and (−1)ⁿ^{₁}⁻ⁿ^{₂}=1, therefore:

$$E = 16 π N [ {\sum }_{{n}_{1} , {n}_{2} = 1 , odd}^{∞} \frac{1}{{n}_{1} {n}_{2}} [ cos ( ( - {n}_{1} + {n}_{2} ) φ ) {I}_{{n}_{1} , {n}_{2} , - {n}_{1} + {n}_{2}} + cos ( ( {n}_{1} + {n}_{2} ) φ ) {I}_{{n}_{1} , {n}_{2} , {n}_{1} + {n}_{2}} ] ] \\ {H}_{BF} ( k ) = \int {d}^{2} ( z - r ) {h}_{BF} ( z - r ) {e}^{- i k \cdot [ z - r ]} = 2 π N {ξ}^{2} {I}_{0 , 0 , 0} ++ 8 π N ξ {\sum }_{n = 1 , even}^{∞} \frac{1}{n} [ sin ( n φ ) - sin ( n φ - n ξ ) ] {I}_{0 , n , n} + 4 π N {\sum }_{{n}_{1} , {n}_{2} = 1}^{∞} \frac{{( - 1 )}^{{n}_{1} - {n}_{2}}}{{n}_{1} {n}_{2}} \{ [ 1 - cos ( {n}_{1} ξ ) - cos ( {n}_{2} ξ ) + cos ( ( {n}_{1} - {n}_{2} ) ξ ) ] cos ( ( - {n}_{1} + {n}_{2} ) φ ) + [ sin ( {n}_{1} ξ ) - sin ( {n}_{2} ξ ) - sin ( ( {n}_{1} - {n}_{2} ) ξ ) ] sin ( ( - {n}_{1} + {n}_{2} ) φ ) \} {I}_{{n}_{1} , {n}_{2} , - {n}_{1} + {n}_{2}} - 4 π N {\sum }_{{n}_{1} , {n}_{2} = 1}^{∞} \frac{{( - 1 )}^{{n}_{2}}}{{n}_{1} {n}_{2}} \{ [ 1 - cos ( {n}_{1} ξ ) - cos ( {n}_{2} ξ ) + cos ( ( {n}_{1} + {n}_{2} ) ξ ) ] cos ( ( {n}_{1} + {n}_{2} ) φ ) + [ - sin ( {n}_{1} ξ ) - sin ( {n}_{2} ξ ) + sin ( ( {n}_{1} + {n}_{2} ) ξ ) ] sin ( ( {n}_{1} + {n}_{2} ) φ ) \} {I}_{{n}_{1} , {n}_{2} , {n}_{1} + {n}_{2}}$$

For the case

$${H}_{BF} ( k ) {\vert}_{ξ = π} = 2 {π}^{3} {NI}_{0 , 0 , 0} + 16 π N [ {\sum }_{{{n}_{1} , {n}_{2} = 1}_{odd}}^{∞} \frac{1}{{n}_{1} {n}_{2}} [ cos ( ( {n}_{2} - {n}_{1} ) φ ) {I}_{{n}_{1} , {n}_{2} , - {n}_{2} - {n}_{1}} + cos ( ( {n}_{1} + {n}_{2} ) φ ) {I}_{{n}_{1} , {n}_{2} , {n}_{1} + {n}_{2}} ] ]$$

### ANNEX A III

### Integration to obtain H_{BF}(k) for ξ=π

We repeat the calculation for ξ=π by starting with the more general form as a check:

$${H}_{BF} ( k ) = N {\sum }_{{n}_{2} = - ∞}^{∞} {\sum }_{{n}_{1} = - ∞}^{∞} \frac{2 {πi}^{2 {n}_{1} - 2 {n}_{2}}}{{n}_{1} {n}_{2}} ( {e}^{i ( {n}_{1} - {n}_{2} ) π} - {e}^{- i {n}_{2} π} - {e}^{i {n}_{1} π} + 1 ) {e}^{i ( {n}_{2} - {n}_{1} ) φ} {I}_{{n}_{1} , {n}_{2} , {n}_{2} - {n}_{1}}$$

Note the identity:

$${e}^{i n π} = cos ( n π ) + i sin ( n π ) = \{ \begin{matrix}1 , & n = even \\ - 1 , & n = odd\end{matrix}$$

and use it for inspecting the n₁=0 and n₂=0 cases:

$$\frac{2 {πi}^{{n}_{1} - 2 {n}_{2}}}{{n}_{1} {n}_{2}} ( {e}^{i ( {n}_{1} - {n}_{2} ) π} - {e}^{- i {n}_{2} π} - {e}^{i {n}_{1} π} + 1 ) == \{ \begin{matrix}2 {π}^{3} & {n}_{1} = 0 , {n}_{2} = 0 \\ 2 π {i}^{- 2 {n}_{2}} \frac{{iπe}^{i {n}_{2} π} - iπ}{{n}_{2}} ; & \{ \begin{matrix}0 ; & {n}_{1} = 0 , {n}_{2} \neq 0 ( even ) \\ \frac{4 {iπ}^{2}}{{n}_{2}} ; & {n}_{1} = 0 , {n}_{2} \neq 0 ( odd )\end{matrix} \\ 2 π {i}^{2 {n}_{2}} \frac{- {iπe}^{i {n}_{1} π} + iπ}{{n}_{1}} ; & \{ \begin{matrix}0 ; & {n}_{2} = 0 , {n}_{1} \neq 0 ( even ) \\ - \frac{4 {iπ}^{2}}{{n}_{1}} ; & {n}_{2} = 0 , {n}_{1} \neq 0 ( odd )\end{matrix} \\ \frac{2 {πi}^{2 {n}_{1} - 2 {n}_{2}}}{{n}_{1} {n}_{2}} ( \begin{matrix}{e}^{i ( {n}_{1} - {n}_{2} ) π} - \\ {e}^{- i {n}_{2} π} - \\ {e}^{i {n}_{1} π} + 1\end{matrix} ) & \{ \begin{matrix}0 ; & {n}_{1} \neq 0 ( even ) , {n}_{2} \neq 0 ( even ) \\ 0 ; & {n}_{1} \neq 0 ( odd ) , {n}_{2} \neq 0 ( even ) \\ 0 ; & {n}_{1} \neq 0 ( even ) , {n}_{2} \neq 0 ( odd ) \\ \frac{8 π}{{n}_{1} {n}_{2}} & {n}_{1} \neq 0 ( odd ) , {n}_{2} \neq 0 ( odd )\end{matrix}\end{matrix}$$

Therefore:

$${H}_{BF} ( k ) = N \{ 2 {π}^{3} {I}_{0 , 0 , 0} + {\sum }_{{n}_{2} = - ∞ ( odd , {n}_{2} \neq 0 )}^{∞} \frac{4 {iπ}^{2}}{{n}_{2}} {e}^{i {n}_{2} φ} {I}_{0 , {n}_{2} , {n}_{2}} - {\sum }_{{n}_{2} = - ∞ ( odd , {n}_{1} \neq 0 )}^{∞} \frac{4 {iπ}^{2}}{{n}_{1}} {e}^{- i {n}_{1} φ} {I}_{{n}_{1} , 0 , - {n}_{1}} + {\sum }_{{n}_{2} = - ∞ ( odd , {n}_{2} \neq 0 )}^{∞} {\sum }_{{n}_{1} = - ∞ ( odd , {n}_{1} \neq 0 )}^{∞} \frac{8 π}{{n}_{1} {n}_{2}} {e}^{i ( {n}_{2} - {n}_{1} ) φ} {I}_{{n}_{1} , {n}_{2} , {n}_{2} - {n}_{1}} \}$$

$${I}_{{n}_{1} , 0 , - {n}_{1}} = {( - 1 )}^{{n}_{1}} {I}_{{n}_{1} , 0 , {n}_{1}} ; {I}_{{n}_{1} , 0 , {n}_{1}} = {I}_{0 , {n}_{1} , , {n}_{1}}$$

The “second term” T₂ becomes:

$${T}_{2} = {\sum }_{{n}_{2} = - ∞ ( odd , {n}_{2} \neq 0 )}^{∞} \frac{4 {iπ}^{2}}{{n}_{2}} {e}^{i {n}_{2} φ} {I}_{0 , {n}_{2} , {n}_{2}} - {\sum }_{{n}_{1} = - ∞ ( odd , {n}_{1} \neq 0 )}^{∞} \frac{4 {iπ}^{2}}{{n}_{1}} {e}^{- i {n}_{1} φ} {I}_{{n}_{1} , 0 , - {n}_{1}} = {\sum }_{{n}_{2} = - ∞ ( odd , {n}_{2} \neq 0 )}^{∞} \frac{4 {iπ}^{2}}{{n}_{2}} [ {e}^{i {n}_{2} φ} {I}_{0 , {n}_{2} , {n}_{2}} - {e}^{- i {n}_{2} φ} {I}_{{n}_{2} , 0 , - {n}_{2}} ] = {\sum }_{{n}_{2} = 1 ( odd )}^{∞} \frac{4 {iπ}^{2}}{{n}_{2}} [ {e}^{i {n}_{2} φ} {I}_{0 , {n}_{2} , {n}_{2}} - {e}^{- i {n}_{2} φ} {I}_{{n}_{2} , 0 , - {n}_{2}} - {e}^{- {i }_{2} φ} {I}_{0 , - {n}_{2} , - {n}_{2}} + {e}^{i {n}_{2} φ} {I}_{- {n}_{2} , 0 , {n}_{2}} ] = {\sum }_{{n}_{2} = 1 ( odd )}^{∞} \frac{4 {iπ}^{2}}{{n}_{2}} [ {e}^{i {n}_{2} φ} {I}_{0 , {n}_{2} , {n}_{2}} - {e}^{- i {n}_{2} φ} {I}_{{n}_{2} , 0 , - {n}_{2}} - {e}^{- i {n }_{2} φ} {I}_{0 , - {n}_{2} , - {n}_{2}} + {e}^{i {n}_{2} φ} {I}_{- {n}_{2} , 0 , {n}_{2}} ] = {\sum }_{{n}_{2} = 1 ( odd )}^{∞} \frac{4 {iπ}^{2}}{{n}_{2}} {I}_{0 , {n}_{2} , {n}_{2}} [ {e}^{i {n}_{2} φ} + {e}^{- i {n}_{2} φ} - {e}^{- i {n}_{2} φ} - {e}^{i {n}_{2} φ} ] = 0 \\ ⇒ \\ {H}_{BF} ( k ) = N \{ 2 {π}^{3} {I}_{0 , 0 , 0} + {\sum }_{{n}_{2} = - ∞ ( odd , {n}_{2} \neq 0 )}^{∞} {\sum }_{{n}_{1} = - ∞ ( odd , {n}_{1} \neq 0 )}^{∞} \frac{8 π}{{n}_{1} {n}_{2}} {e}^{i ( {n}_{2} - {n}_{1} ) φ} {I}_{{n}_{1} , {n}_{2} , {n}_{2} - {n}_{1}} \}$$

For the double sum term we note that for the case:

$${n}_{2} = - {n}_{2} and {n}_{1} = - {n}_{1} , \\ {n}_{1} ( odd ) , {n}_{2} ( odd ) {\text{:} [ {J}_{- n} ( x ) = {( - 1 )}^{n} {J}_{n} ( x ) ]}^{14}$$

$${I}_{- {n}_{1} , - {n}_{2} , - {n}_{2} + {n}_{1}} = {( - 1 )}^{{n}_{1} + {n}_{2} + {n}_{2} - {n}_{1}} {I}_{{n}_{1} , {n}_{2} , {n}_{2} - {n}_{1}} = {( - 1 )}^{2 {n}_{2}} {I}_{{n}_{1} , {n}_{2} , {n}_{2} - {n}_{1}} = {I}_{{n}_{1} , {n}_{2} , {n}_{2} - {n}_{1}} = > \frac{8 π}{{n}_{1} {n}_{2}} {e}^{i ( {n}_{2} - {n}_{1} ) φ} {I}_{{n}_{1} , {n}_{2} , {n}_{2} - {n}_{1}} + \frac{8 π}{( - {n}_{1} ) ( - {n}_{2} )} {e}^{i ( {n}_{2} - {n}_{1} ) φ} {I}_{- {n}_{1} , - {n}_{2} , - {n}_{2} + {n}_{1}} = \frac{8 π}{{n}_{1} {n}_{2}} {I}_{{n}_{1} , {n}_{2} , {n}_{2} - {n}_{1}} ( {e}^{i ( {n}_{2} - {n}_{1} ) φ} + {e}^{- i ( {n}_{2} - {n}_{1} ) φ} ) = \frac{16 π}{{n}_{1} {n}_{2}} {I}_{{n}_{1} , {n}_{2} , {n}_{2} - {n}_{1}} cos ( ( {n}_{2} - {n}_{1} ) φ )$$

¹⁴ http://en.wikipedia.org/wiki/Bessel_function
and we note that for the case:

$$( {n}_{2} = {n}_{2} and {n}_{1} = - {n}_{1} ) + ( {n}_{2} = - {n}_{2} and {n}_{1} = {n}_{1} ) , \\ {n}_{1} ( odd ) , {n}_{2} ( odd ) \text{:}$$

$${I}_{- {n}_{1} , {n}_{2} , ( {n}_{2} + {n}_{1} )} = {( - 1 )}^{- {n}_{2} + {n}_{1} - {n}_{2} - {n}_{1}} {I}_{{n}_{1} , - {n}_{2} , - ( {n}_{2} + {n}_{1} )} = {( - 1 )}^{- 2 {n}_{2}} {I}_{{n}_{1} , - {n}_{2} , - ( {n}_{2} + {n}_{1} )} = {I}_{{n}_{1} , - {n}_{2} , - ( {n}_{2} + {n}_{1} )} \\ ⇒ \\ \frac{8 π}{( - {n}_{1} ) ( {n}_{2} )} {e}^{i ( {n}_{2} + {n}_{1} ) φ} {I}_{- {n}_{1} , {n}_{2} , {n}_{2} + {n}_{1}} + \frac{8 π}{( {n}_{1} ) ( - {n}_{2} )} {e}^{- i ( {n}_{2} + {n}_{1} ) φ} {I}_{{n}_{1} , - {n}_{2} , - ( {n}_{2} + {n}_{1} )} = - \frac{8 π}{{n}_{1} {n}_{2}} ( {e}^{i ( {n}_{2} + {n}_{1} ) φ} + {e}^{- i ( {n}_{2} + {n}_{1} ) φ} ) {I}_{- {n}_{1} , {n}_{2} , ( {n}_{2} + {n}_{1} )} = - \frac{16 π}{{n}_{1} {n}_{2}} cos ( {n}_{2} + {n}_{1} ) {I}_{- {n}_{1} , {n}_{2} , ( {n}_{2} + {n}_{1} )} = \frac{16 π}{{n}_{1} {n}_{2}} cos ( {n}_{2} + {n}_{1} ) {I}_{{n}_{1} , {n}_{2} , {n}_{2} + {n}_{1}} \\ ⇒$$

We can therefore replace the [−∞ to ∞] summations by [1 to ∞] and arrive at:

$${H}_{BF} ( k ) = N \{ 2 {π}^{3} {I}_{0 , 0 , 0} ++ {\sum }_{{n}_{2} = 1 ( odd ) , {n}_{1} = 1 ( odd )}^{∞} \frac{16 π}{{n}_{1} {n}_{2}} [ cos ( ( {n}_{2} - {n}_{1} ) φ ) {I}_{{n}_{1} , {n}_{2} , {n}_{2} - {n}_{1}} + cos ( ( {n}_{2} + {n}_{1} ) φ ) {I}_{{n}_{1} , {n}_{2} , {n}_{2} + {n}_{1}} ] \} \qquad ( 23 )$$

### ANNEX B

### Diffraction Tomography Algorithm for the Limited View Hemi-Spherical Aperture

A new derivation of a three-dimensional DT based on a three-dimensional beamforming (BF) algorithm is discussed, as an alternative approach to standard DT algorithms such as the filtered backpropagation method¹⁵. ¹⁵ Devaney, A. J. 1982, “A filtered backpropagation algorithm for diffraction tomography”, Ultrason. Imaging 4, 336-350.

We assume that the scattering problem is described by a scalar wave field, ψ, solution to

Hψ(r,k₀\hat{r}₀,ω)=−O(r,ω)ψ(r,k₀\hat{r}₀,ω) (1)

where H is the Helmholtz operator (∇²+k₀²), k₀ is the background wavenumber (2π/λ), \hat{r}₀ specifies the direction of an incident plane wave that illuminates the object and ω is the angular frequency. The unit vector \hat{r}₀ is defined by the angles θₜ and φₜ of a spherical coordinate systemError! Reference source not found.

The object is described by the so-called Object Function that depends on the type of wave field used to probe the object: for electromagnetic wave sensing, it is related to the index of refraction¹⁶, n(r,ω), through the relation O(r)=k₂⁰[n²(r,ω)−1], and for acoustic waves, it is linked to the speed of sound and the attenuation coefficient¹⁷. In particular, for a lossless object

$$o ( r , ω ) = {k}_{0}^{2} [ {( \frac{{c}_{0}}{c ( r , ω )} )}^{2} - 1 ] \qquad ( 2 )$$

where c₀ is the sound speed of the homogeneous background in which the object is immersed and c(r,ω) is the local sound speed inside the object. The dependence of the object function on ω is because of dispersion and energy dissipation phenomena. The analysis performed in the rest of this section will consider monochromatic wave fields; therefore, the explicit dependence on ω is omitted. ¹⁶ Born, M. & Wolf, E. 1999 Principles of optics. Cambridge, UK: Cambridge University Press.¹⁷ Kak, A. C. & Slaney, M. 1988 Principles of computerized tomographic imaging. New York, N.Y.: IEEE Press.

### Three-Dimensional Beamforming Algorithm Over a Semi-Sphere

Let us assume that the scattering amplitude, ƒ(θᵣ, θₜ, φᵣ, φₜ), can be measured as a continuous function of the illumination and detection directions, i.e. θᵣ, θₜε[0,π] and φᵣ, φₜε[0,π] for a semi-sphere, (note that for a full sphere φᵣ, φₜε[0,2π]), these angles being the receive and transmit directions in a spherical coordinate system respectively. In principle, this could be achieved with the semi-spherical array of transceivers that surrounds the object.

Standard BF produces the image of an object at a point, z, of the image space by focusing an incident beam at r and z in the object space. The resulting scattered field is subsequently phase shifted and integrated over the aperture of the array, so that only the contributions to the scattered field from the focal point are added coherently. This two-step process is obtained by means of the BF functional

ℑ_{BF}=∫₀^{π}dφᵣ∫₀^{π}dθᵣ sin θᵣ∫₀^{π}dφₜ∫₀^{π}dθₜ sin θₜ×exp[ik₀û(θᵣ,φᵣ)·z]ƒ(θᵣ,θₜ,φᵣ,φₜ)exp[ik₀û(θₜ,φₜ)·z] (3)

where û is the unit vector associated with the angles θ and φ. As discussed by¹⁸ for the two-dimensional case, the second exponential in equation (III) represents focusing in transmission, whereas the first corresponds to the focusing of the received scattered field. The point spread function (PSF) associated with the functional (III) can be obtained by considering the image of a point scatterer at position r. In this case, the free-scattering amplitude is

ƒ_{free}(θᵣ,θₜ,φᵣ,φₜ)=exp{−ik₀[û(θₜ,φₜ)+û(θᵣ,φᵣ)]·r} (4)

and the PSF reads

h_{BF}=∫₀^{π}dφᵣ∫₀^{π}dθᵣ sin θᵣ∫₀^{π}dφₜ∫₀^{π}dθₜ sin θₜ×exp{ik₀û(θᵣ,φᵣ)·[z−r]}exp{ik₀û(θₜ,φₜ)·[z−r]} (5)

¹⁸ Simonetti, F. & Huang, L. 2008, “From beamforming to diffraction tomography”, J. Appl. Phys. 103, 103 110.

Spherical waves expansion:

$$exp \{ i {k}_{0} \hat{u} ( {θ}_{r} , {φ}_{r} ) \cdot [ z - r ] \} = {\sum }_{l = 0}^{∞} {i}^{l} ( 2 l + 1 ) {j}_{l} ( {k}_{0} \vert z - r \vert ) {P}_{l} [ cos ( angle ( \hat{u} ( {θ}_{r} , {φ}_{r} ) \cdot ( z - r ) ) ) ]$$

where jₗ is the spherical Bessel function of the order l and Pₗ are Legendre Polynomials.

$${\int }_{0}^{π} d {φ}_{r} {\int }_{0}^{π} d {θ}_{r} sin {θ}_{r} exp \{ i {k}_{0} \hat{u} ( {θ}_{r} , {φ}_{r} ) \cdot [ z - r ] \} == {\int }_{0}^{π} d {φ}_{r} {\int }_{0}^{π} d {θ}_{r} sin {θ}_{r} \{ {j}_{0} ( {k}_{0} \vert z - r \vert ) + {\sum }_{l = 1}^{∞} {i}^{l} ( 2 l + 1 ) {j}_{l} ( {k}_{0} \vert z - r \vert ) {P}_{l} [ cos ( angle ( \hat{u} ( {θ}_{r} , {φ}_{r} ) \cdot ( z - r ) ) ) ] \} =$$

Define the angles representing z−r as θ′, φ′. The summation formula for the Spherical Harmonics is now intorduced¹⁹:

$${P}_{l} ( cos γ ) = \frac{4 π}{2 l + 1} {\sum }_{m = - 1}^{l} {Y}_{lm}^{*} ( {θ}_{r} , {φ}_{r} ) {Y}_{lm} ( {θ}^{′} , {φ}^{′} )$$

where
cos γ=cos θ cos θ′+sin θ sin θ′ cos(φ−φ′) ¹⁹ http://farside.ph.utexas.edu/teaching/jk1/lectures/node102.html

Therefore, the integration over θᵣ and φᵣ becomes:

$${\int }_{0}^{π} d {φ}_{r} {\int }_{0}^{π} d {θ}_{r} sin {θ}_{r} {Y}_{lm}^{*} ( {θ}_{r} , {φ}_{r} )$$

$${Y}_{lm}^{*} ( {θ}_{r} , {φ}_{r} ) ≡ \{ \begin{matrix}{K}_{l}^{m} {P}_{l}^{\vert m \vert} ( cos {θ}_{r} ) cos ( \vert m \vert {φ}_{r} ) & if m \geq 0 \\ {K}_{l}^{m} {P}_{l}^{\vert m \vert} ( cos {θ}_{r} ) sin ( \vert m \vert {φ}_{r} ) & if m < 0\end{matrix}$$

where

$${K}_{l}^{m} = \sqrt{\frac{( 2 l + 1 ) ( l - m ) !}{4 π ( l + m ) !}}$$

As is well known, from the above definition it follows that the spherical Harmonics are separable in θᵣ, φᵣ.

We now define the following integrals²⁰

$${\int }_{θ -}^{θ +} {P}_{l}^{\vert m \vert} ( cos θ ) sin θ d θ = {\int }_{z -}^{z +} {P}_{l}^{\vert m \vert} ( z ) d z = {\hat{P}}_{l}^{\vert m \vert} ( {θ}_{-} , {θ}_{+} )$$

where z=cos θ, and z_{±}=cos θ_{±}. ²⁰ W. Jarosz, N. Carr & H. W. Jensen, “Importance Sampling Spherical Harmonics”, Journal compilation, 2008, The Eurographics Association and Blackwell Publishing Ltd.

We also define

$${Φ}^{m} ( φ ) = \{ \begin{matrix}cos ( m φ ) & if m \geq 0 \\ sin ( \vert m \vert φ ) & if m < 0\end{matrix} \\ {\hat{Φ}}^{m} ( φ ) = \int {Φ}^{m} ( φ ) d φ = \frac{1}{m} \{ \begin{matrix}sin ( m φ ) & if m \geq 0 \\ cos ( m φ ) & if m < 0\end{matrix} \\ {\hat{Φ}}^{m} ( {φ}_{-} , {φ}_{+} ) = {\int }_{φ -}^{φ +} {Φ}^{m} ( φ ) d φ$$

And with \hat{φ}^{m} and \hat{P}ₗ^{|m|} we define:

Ŷₗₘ(θ₋,θ₊,φ₋,φ₊)=Kₗ^{m}\hat{P}ₗ^{|m|}(θ₋,θ₊)\hat{φ}^{m}(φ₋,φ₊)

We now go back to the beamforming terms and look at:

$${g}_{r} ( z - r ) = {\int }_{0}^{π} d {φ}_{r} {\int }_{0}^{π} d {θ}_{r} sin {θ}_{r} exp \{ i {k}_{0} \hat{u} ( {θ}_{r} , {φ}_{r} ) \cdot [ z - r ] \} == {\int }_{0}^{π} d {φ}_{r} {\int }_{0}^{π} d {θ}_{r} sin {θ}_{r} \{ {j}_{0} ( {k}_{0} \vert z - r \vert ) + {\sum }_{l = 1}^{∞} {i}^{l} ( 2 l + 1 ) {j}_{l} ( {k}_{0} \vert z - r \vert ) \frac{4 π}{2 l + 1} {\sum }_{m = - 1}^{l} {Y}_{lm}^{*} ( {θ}_{r} , {φ}_{r} ) {Y}_{lm} ( {θ}^{′} , {φ}^{′} ) \} == 2 π {j}_{0} ( {k}_{0} \vert z - r \vert ) + {\sum }_{l = 1}^{∞} {i}^{l} ( 2 l + 1 ) {j}_{l} ( {k}_{0} \vert z - r \vert ) \frac{4 π}{2 l + 1} {\sum }_{m = - 1}^{l} {\hat{Y}}_{lm}^{*} ( 0 , π , 0 , n ) {Y}_{lm} ( {θ}^{′} , {φ}^{′} ) = {\sum }_{l = 0}^{∞} {\sum }_{m = - l}^{l} {A}_{l}^{m} {j}_{l} ( {k}_{0} \vert z - r \vert ) {Y}_{lm} ( {θ}^{′} , {φ}^{′} )$$

where

$${A}_{l}^{m} = {i}^{l} ( 2 l + 1 ) \frac{4 π}{2 l + 1} {\hat{Y}}_{lm}^{*} ( 0 , π , 0 , n ) = 4 π {i}^{l} {\hat{Y}}_{lm}^{*} ( 0 , π , 0 , n )$$

The same result is obtained for the transmit angles quantity gₜ(z−r), so we now calculate the three-dimensional Fourier transform H_{BF}(k) of h_{BF}(z−r) given by

$${h}_{BF} ( z - r ) = {g}_{r} ( z - r ) {g}_{t} ( z - r ) = ( {\sum }_{l = 0}^{∞} {\sum }_{m = - l}^{l} {A}_{l}^{m} {j}_{l} ( {k}_{0} \vert z - r \vert ) {Y}_{lm} ( {θ}^{′} , {φ}^{′} ) ) \times ( {\sum }_{{l}^{′} = 0}^{∞} {\sum }_{{m}^{′} = - {l}^{′}}^{{l}^{′}} {A}_{{l}^{′}}^{{m}^{′}} {j}_{{l}^{′}} ( {k}_{0} \vert z - r \vert ) {Y}_{{l}^{′} {m}^{′}} ( {θ}^{′} , {φ}^{′} ) ) \\ {H}_{BF} ( k ) = {\int }_{- ∞}^{∞} {d}^{3} {rg}_{r} ( z - r ) {g}_{t} ( z - r ) {e}^{- i k \cdot [ z - r ]} == {\int }_{- ∞}^{∞} {d}^{3} r {e}^{- i k \cdot [ z - r ]} ( {\sum }_{l = 0}^{∞} {\sum }_{m = - l}^{l} {A}_{l}^{m} {j}_{l} ( {k}_{0} \vert z - r \vert ) {Y}_{lm} ( {θ}^{′} , {φ}^{′} ) ) \times ( {\sum }_{{l}^{′} = 0}^{∞} {\sum }_{{m}^{′} = - {l}^{′}}^{{l}^{′}} {A}_{{l}^{′}}^{{m}^{′}} {j}_{{l}^{′}} ( {k}_{0} \vert z - r \vert ) {Y}_{{l}^{′} {m}^{′}} ( {θ}^{′} , {φ}^{′} ) ) \qquad ( 6 )$$

We use the expansion in spherical waves once again:

exp{−ik·[z−r]}=Σₗ₌₀^{∞}i^{l}(2l+1)jₗ(k₀|z−r|)Pₗ[cos(angle(k·(z−r)))]

Denoting by θ,φ the angles of k and as before θ′,φ′ are angles of −r, (addition theorem):

$$exp \{ - i k \cdot [ z - r ] \} = {\sum }_{{l}^{″} = 0}^{∞} {i}^{{l}^{″}} ( 2 {l}^{″} + 1 ) {j}_{{l}^{″}} ( \vert k \vert \vert z - r \vert ) \frac{4 π}{2 {l}^{″} + 1} {\sum }_{{m}^{″} = - {l}^{″}}^{{l}^{″}} {Y}_{{l}^{″} {m}^{″}}^{*} ( θ , φ ) {Y}_{{l}^{″} {m}^{″}} ( {θ}^{′} , {φ}^{′} ) \\ {H}_{BF} = {\int }_{0}^{∞} {r}^{2} d r {\int }_{0}^{π} sin {θ}^{′} d {θ}^{′} {\int }_{0}^{2 π} d {φ}^{′} ( {\sum }_{{l}^{″} = 0}^{∞} {i}^{{l}^{″}} ( 2 {l}^{″} + 1 ) {j}_{{l}^{″}} ( \vert k \vert \vert z - r \vert ) \frac{4 π}{2 {l}^{″} + 1} {\sum }_{{m}^{″} = - {l}^{″}}^{{l}^{″}} {Y}_{{l}^{″} {m}^{″}}^{*} ( θ , φ ) {Y}_{{l}^{″} {m}^{″}} ( {θ}^{′} , {φ}^{′} ) ) \times ( {\sum }_{l = 0}^{∞} {\sum }_{m = - l}^{l} {A}_{l}^{m} {j}_{l} ( {k}_{0} \vert z - r \vert ) {Y}_{lm} ( {θ}^{′} , {φ}^{′} ) ) \times ( {\sum }_{{l}^{′} = 0}^{∞} {\sum }_{{m}^{′} = - {l}^{′}}^{{l}^{′}} {A}_{{l}^{′}}^{{m}^{′}} {j}_{{l}^{′}} ( {k}_{0} \vert z - r \vert ) {Y}_{{l}^{′} {m}^{′}} ( {θ}^{′} , {φ}^{′} ) ) \\ {H}_{BF} ( k ) = {\sum }_{l , m , {l}^{′} , {m}^{′} , {l}^{″} , {m}^{″}} {B}_{{l}^{″}} {C}_{l , m , {l}^{′} , {m}^{′} , {l}^{″} , {m}^{″}} {Y}_{{l}^{″} {m}^{″}}^{*} ( θ , φ ) \times {\int }_{0}^{∞} {r}^{2} d r {j}_{{l}^{″}} ( \vert k \vert \vert z - r \vert ) {j}_{{l}^{′}} ( {k}_{0} \vert z - r \vert ) {j}_{l} ( {k}_{0} \vert z - r \vert ) \qquad ( 7 )$$

where C_{l,m,l′,m′,l″,m″}=∫₀^{π} sin θ′ dθ′∫₀^{2π}dφ′Yₗₘ(θ′,φ′)Y_{l′m′}(θ′,φ′)Y_{l″m″}(θ′,φ′) and B_{l″}=4πi^{l″}.

We now focus on the integral of a product of 3 spherical Bessel functions²¹:

$$I ( {λ}_{1} , {λ}_{2} , {λ}_{3} ; {k}_{1} , {k}_{2} , {k}_{3} ) ≡ {\int }_{0}^{∞} {r}^{2} d r {j}_{{λ}_{1}} ( {k}_{1} r ) {j}_{{λ}_{2}} ( ( {k}_{2} r ) {j}_{{λ}_{3}} ( ( {k}_{3} r ) \\ I ( {λ}_{1} , {λ}_{2} , {λ}_{3} ; {k}_{1} , {k}_{2} , {k}_{3} ) = \frac{πβ ( Δ )}{4 {k}_{1} {k}_{2} {k}_{3}} {{i}^{{λ}_{1} + {λ}_{2} - {λ}_{3}} ( 2 {λ}_{3} + 1 )}^{\frac{1}{2}} {( \frac{{k}_{1}}{{k}_{3}} )}^{{λ}_{3}} \cdot {( \begin{matrix}{λ}_{1} & {λ}_{2} & {λ}_{3} \\ 0 & 0 & 0\end{matrix} )}^{- 1} {\sum }_{L = 0}^{{λ}_{3}} {( \begin{matrix}2 {λ}_{3} \\ 2 L\end{matrix} )}^{\frac{1}{2}} {( \frac{{k}_{2}}{{k}_{1}} )}^{L} {\sum }_{l} ( 2 l + 1 ) ( \begin{matrix}{λ}_{1} & {λ}_{3} - L & l \\ 0 & 0 & 0\end{matrix} ) \cdot ( \begin{matrix}{λ}_{2} & L & l \\ 0 & 0 & 0\end{matrix} ) \{ \begin{matrix}{λ}_{1} & {λ}_{2} & {λ}_{3} \\ L & {λ}_{3} - L & l\end{matrix} \} {P}_{l} ( Δ )$$

where
|k₁−k₂|≦k₃≦k₁+k₂(closed triangle, angular momentum conservation)

$$Δ = \frac{{k}_{1}^{2} + {k}_{2}^{2} - {k}_{3}^{2}}{2 {k}_{1} {k}_{2}}$$

Δ lies between ±1 and is the cosine of the angle between \hat{k}₁ and \hat{k}₂ in the triangle formed by k₁, k₂ and k₃. ²¹ R Mehremt, 3 T Londergant and M H Macfarlanet, “Analytic expressions for integrals of products of spherical Bessel functions”, J. Phys. A: Math. Gen. 24(1991) 1435-1453.

The quotation for I(λ₁, λ₂, λ₃; k₁, k₂, k₃) is valid for all real Δ, including values outside the limited range −1≦Δ≦1, with correct account taken of the jump discontinuities at Δ=±1, through the introduction of the function

β(Δ)=∂(1−Δ)∂(1+Δ)

where ∂(y) is a modified step function:

$$ϑ ( y ) = \{ \begin{matrix}0 & y < 0 \\ \frac{1}{2} & y = 0 \\ 1 & y > 0\end{matrix}$$

$$( \begin{matrix}{λ}_{1} & {λ}_{2} & {λ}_{3} \\ 0 & 0 & 0\end{matrix} )$$

is the is the Wigner 3-j symbol²² from which the angular momentum triangle is deduced, and recoupling of three angular momenta involves the 6-j symbol

$$\{ \begin{matrix}{j}_{1} & {j}_{2} & {j}_{3} \\ {j}_{4} & {j}_{5} & {j}_{6}\end{matrix} \} .$$

²² Edmonds A R 1957 Angular Momentum in Quantum Mechanics (Princeton: Princeton University Academic Press)

Our integral is:

$$I ( l , {l}^{′} , {l}^{″} ; k , {k}_{0} , {k}_{0} ) = {\int }_{0}^{∞} {r}^{2} d {rj}_{{l}^{″}} ( \vert k \vert \vert z - r \vert ) {j}_{{l}^{′}} ( {k}_{0} \vert z - r \vert ) {j}_{l} ( {k}_{0} \vert z - r \vert )$$

We therefore arrived at an analytic formula for H_{BF} of the semi-sphere, and in fact other limited view angles. Next we consider the value of Δ for our case:

With k₁=|k|, and k₂=k₃=k₀ we get:

$$Δ = \frac{{\vert k \vert}^{2} + {k}_{0}^{2} - {k}_{0}^{2}}{2 \vert k \vert {k}_{0}} = \frac{\vert k \vert}{2 {k}_{0}}$$

As −1≦Δ≦1, we get the low pass filtering:

$${H}_{BF} = g ( k ) Π ( \vert k \vert ) , where Π ( \vert k \vert ) = \{ \begin{matrix}1 & \vert k \vert < 2 {k}_{0} \\ 0 & \vert k \vert > 2 {k}_{0}\end{matrix} \qquad ( 8 )$$

The DT problem consists of reconstructing the function O(r) from a set of scattering experiments. For this purpose, it is convenient to introduce the representation of the object function in the spatial frequency domain, K-space, which is obtained by performing the three-dimensional Fourier transform of O(r)

$$\tilde{O} ( k ) = {\int }_{- ∞}^{∞} {d}^{3} r O ( r ) {e}^{- i k \cdot r} \qquad ( 9 )$$

We now look at the beamforming image:

$${𝒥}_{BF} = {\int }_{- ∞}^{∞} d {r}_{1} {\int }_{- ∞}^{∞} d {r}_{2} {\int }_{- ∞}^{∞} d {r}_{3} O ( r ) h ( \vert z - r \vert ) \qquad ( 10 )$$

that in the spatial frequency domain reads

I_{BF}(k)=Õ(k)H_{BF}(k)=g(k)Õ(k)Π(|k|) (11)

While DT over the entire sphere leads to the low-pass-filtered image, Õ(k)Π(|k|), the new BF algorithm introduces a distortion that is described by the additional filter g(k). As a result, the DT image can be obtained from the BF image by applying the filter

$$\frac{1}{g ( k )}$$

to the BF image. Again, this is an alternative approach to other DT algorithms²³. ²³ Reference of footnote 3

With no loss of generality we can choose the vector k parallel to the z axis, i.e θ=0 and cos θ=1.

For this case,

$${Y}_{{l}^{″} {m}^{″}} ( 0 , φ ) = \{ \begin{matrix}1 , & {m}^{″} = 0 for all {l}^{″} \\ 0 , & {m}^{″} \neq 0 for all {l}^{″}\end{matrix}$$

so the equation becomes independent of angles, i.e depends on |k|only:

$${H}_{BF} ( k ) = {\sum }_{l , m , {l}^{′} , {m}^{′} , {l}^{″}} {B}_{{l}^{″}} {C}_{l , m , {l}^{′} , {m}^{′} , {l}^{″} , 0} \times {\int }_{0}^{∞} {r}^{2} d r {j}_{{l}^{″}} ( \vert k \vert \vert z - r \vert ) {j}_{{i}^{′}} ( {k}_{0} \vert z - r \vert ) {j}_{l} ( {k}_{0} \vert z - r \vert ) = {H}_{BF} ( \vert k \vert ) \qquad ( 12 )$$

where again C_{l,m,l′,m′,l″,m″}=∫₀^{π} sin θ′ dθ′∫₀^{2π}dφ′Yₗₘ(θ′,φ′)Y_{l′m′}(θ′,φ′)Y_{l″m″}(θ′,φ′) and B_{l″}=4πi^{l″}.

Therefore, the filter function ƒ(k) becomes a function of |k|only, ƒ(|k|). As

$$Δ = \frac{\vert k \vert}{2 {k}_{0}} ,$$

the filter ƒ(|k|) in this particular coordinate choice becomes a sum over legendre polynomials in

$$\frac{\vert k \vert}{2 {k}_{0}}$$

$$f ( \vert k \vert ) = {\sum }_{n} {M}_{n} {P}_{n} ( \frac{\vert k \vert}{2 {k}_{0}} )$$

| n | Legendre Polynomials ${P}_{n} ( x ) ; x = \frac{\vert k \vert}{2 {k}_{0}}$ |
|---|---|
| 0 | 1 |
| 1 | x |
| 2 | $\frac{1}{2} ( 3 {x}^{2} - 1 )$ |
| 3 | $\frac{1}{2} ( 5 {x}^{3} - 3 x )$ |
| 4 | $\frac{1}{8} ( 35 {x}^{4} - 30 {x}^{2} + 3 )$ |
| . . . | . . . |
| n | ${P}_{n} ( x ) = {( - 1 )}^{n} {\sum }_{k = 0}^{n} ( \begin{matrix}n \\ k\end{matrix} ) ( \begin{matrix}n + k \\ k\end{matrix} ) {( - x )}^{k}$ |

The summation over n denotes symbolically the multiple indices that need to be summed over. Note that the cooeficients Mₙ in the summation over n in the above equation for ƒ(|k|) are known, many of which vanish through, for example, the values of the 3-j and 6-j symbols of the multiple indices in this symbolically denoted summation.

1. A system for limited view ultrasound imaging of a 2D section or a 3D volume of a body part comprising:
(a) one or more ultrasound sensors, the ultrasound sensors being configured to be spatially or temporally arrayed in an array selected from:
(i) a limited view circular arc having a central angle ξ, ξ satisfying 0<ξ<2π, the ultrasound sensors generating a plurality of amplitudes ƒ(φᵣ, φₜ), where ƒ(φᵣ, φₜ) is an amplitude of ultrasound radiation in a direction forming an angle φᵣ with a fixed radius of the limited view circular arc when the body part is probed with incident radiation from a direction forming an angle φₜ with the fixed radius; wherein 0<φr,φt<ξ;
(ii) a concave surface, the ultrasound sensors generating a plurality of amplitudes ƒ(θᵣ, θₜ, φᵣ, φₜ), where ƒ(θᵣ, θₜ, φᵣ, φₜ) is an amplitude of ultrasound radiation when the body part is probed from a transmit direction determined by angles θₜ, φₜ and a receive direction determined by angles θᵣ, φᵣ wherein θᵣ,φₜε[0,π] and φᵣ, φₜε[0,π];
(b) a processor configured to:
calculate from the ƒ(φᵣ, φₜ) or the ƒ(θᵣ, θₜ, φᵣ, φₜ) a beam forming (BF) functional;
calculate free amplitudes ƒ_{free}(φᵣ, φₜ) or ƒ_{free}(θᵣ, θₜ, φᵣ, φₜ);
calculate from the free amplitudes ƒ_{free}(φᵣ φₜ) or ƒ_{free}(θᵣ, θₜ, φᵣ, φₜ) a point spread function (PSF);
calculate a filter g(k) from the Fourier transform H_{BF}(k) of the PSF;
calculate a Fourier transform I_{BF}(k), of the BF functional;
divide I_{BF}(k), by the filter g(k) to yield Õ(k)Π(|k|); and
generate an image of the 2D section or the 3D volume of the body part using the Õ(k)Π(|k|).

2. The system according to claim 1 further comprising scanning device including a dome shaped structure wherein the ultrasound sensors are configured to be spatially or temporally arrayed over at least a portion of the dome structure.

3. The system according to claim 2 wherein the dome shaped structure is configured to be placed over a breast of a female individual.

4. The system according to claim 2 wherein the dome shaped structure includes a layer formed from an acoustically transparent material.

5. The system according to claim 2 comprising one or more C-Arm-tomography-sensors and one or more 2D-array-sensors.

6. The system according to claim 2 wherein the sensors are connected to a step-motor-assembly configured to drive the ultrasound sensors over the scanning device.

7. The system according to claim 6 wherein the step-motor-assembly includes a motor, an encoder, a processor, an indexer and a driver.

8. The system according to claim 5 wherein the C-arm-tomography-transducer is moved along a circular-track.

9. The system according to claim 1 comprising a display device and wherein the processor is configured to display on the image on the display device.

10. The system according to claim 9 wherein the processor is further configured to superimpose on a displayed image one or more B-Mode compounded images or tomography images.

11. The system according to claim 1 further comprising a garment configured to be worn by an individual over the body part, the garment comprising a layer formed from a thermo-responsive-acoustic-transparent-polymer, the thermo-responsive-acoustic-transparent-polymer being in a first viscous state at a first temperature below 37° C. and in a second viscous state at a second temperature above 37° C., the second viscous state having a viscosity above a viscosity of the first viscous state.

12. The system according to claim 11 wherein the garment is a bra.

13. The system according to claim 11 further comprising a chair wherein the scanning device is positioned in the chair with the dome in an adjustable orientation including an inverted orientation.

14. The system according to claim 11 wherein the thermo-responsive-acoustic-transparent-polymer layer is harder at an outer surface as compared to an inner surface that is in contact with the body part.

15. The system according to claim 1 wherein the dome comprises one or more holes configured to receive a biopsy needle.

16. A garment for use in the system of claim 11, the garment configured to be worn by an individual over the body part, the garment comprising a layer formed from a thermo-responsive-acoustic-transparent-polymer, being in a first viscous state at a first temperature below 37° C. and in a second viscous state at a second temperature above 37° C., the second viscous state having a viscosity above a viscosity of the first viscous state.

17. A chair for use in the system of claim 13, wherein the scanning device is positioned in the chair with the dome in an adjustable orientation including an inverted orientation.

18. The system according to claim 1 comprising a 2D array of ultrasound sensors mechanically coupled to a C-arm tomographic arc or to the concave surface and wherein the generated image is a real-time 3D image.

19. A method for limited view ultrasound imaging of a 2D section or a 3D volume of a body part comprising:
(a) providing one or more ultrasound sensors, the ultrasound sensors being configured to be spatially or temporally arrayed in an array selected from:
(i) a limited view circular arc having a central angle ξ, ξ satisfying 0<ξ<2π, the ultrasound sensors generating a plurality of amplitudes ƒ(φᵣ, φₜ), where ƒ(φᵣ, φₜ) is an amplitude of ultrasound radiation in a direction forming an angle φᵣ with a fixed radius of the limited view circular arc when the planar section is probed with incident radiation from a direction forming an angle φₜ with the fixed radius; wherein 0<φᵣ,φₜ<ξ.
(ii) a concave surface, the ultrasound sensors generating a plurality of amplitudes ƒ(θᵣ, θₜ, φᵣ, φₜ), where ƒ(θᵣ, θₜ, φᵣ, φₜ) is an amplitude of ultrasound radiation when the body part is probed from a transmit direction determined by angles θₜ, φₜ and a receive direction determined by angles θᵣ φᵣ the angles satisfying θᵣ, θₜε[0,π] and φᵣ,φₜε[0,π],
(b) calculating from the ƒ(φᵣ, φₜ) or the ƒ(θᵣ, θₜ, φᵣ, φₜ) a beam forming (BF) functional;
(c) calculating free amplitudes ƒ_{free}(φᵣ, φₜ) or the ƒ_{free}(θᵣ, θₜ, φᵣ, φₜ)
(d) calculating from the free amplitudes ƒ_{free}(φᵣφₜ) or the ƒ_{free}(θᵣ, θₜ, φᵣ,φₜ) a point spread function (PSF);
(e) calculating a filter g(k) from the Fourier transform H_{BF}(k) of the PSF;
(f) calculating a Fourier transform I_{BF}(k), of the BF functional;
(g) dividing I_{BF}(k), by the filter g(k) to yield Õ(k)Π(|k|); and
(h) generating an image of 2D section or the 3D volume of the body part using the Õ(k)Π(|k|).

20. The method according to claim 19 further comprising spatially or temporally arraying the ultrasound sensors over at least a portion of the dome structure.

21. The method according to claim 20 wherein the body part is a breast.

22. The method according to claim 20 wherein the dome shaped structure includes a layer formed from an acoustically transparent material.

23. The method according to claim 19 further comprising display the image on a display device.

24. The method according to claim 23 further comprising superimposing on a displayed image one or more B-Mode compounded images or tomography images.

25. The method according to claim 19 placing a garment on an individual over the body part, the garment comprising a layer formed from a thermo-responsive-acoustic-transparent-polymer, the thereto-responsive-acoustic-transparent-polymer being in a first viscous state at a first temperature below 37° C. and in a second viscous state at a second temperature above 37° C., the second viscous state having a viscosity above a viscosity of the first viscous state.

26. The method according to claim 25 wherein the garment is a bra.

27. The method according to claim 19 wherein scanning device is positioned in a chair with the dome in adjustable orientation including an inverted orientation, and the method further comprises placing the body part in the dome.

28. The method according to claim 27 further comprising inserting into the inverted dome a thermo-responsive-acoustic-transparent-polymer being in a first viscous state at a first temperature below 37° C. and in a second viscous state at a second temperature above 37° C., the second viscous state having a viscosity above a viscosity of the first viscous state.

29. The method according to claim 25 wherein the thermo-responsive-acoustic-transparent-polymer layer is harder at an outer surface as compared to an inner surface that is in contact with the body part.

30. The method according to claim 19 further comprising inserting a biopsy needle in a hole in the dome structure and obtaining a biopsy.

31. The method according to claim 19 wherein a 2D array of ultrasound sensors is mechanically coupled to a C-arm tomographic arc or to the concave surface and the method further provides generating a real-time 3D image.

32. The method according to claim 31 further comprising guiding a surgical or procedure tool through the body part.
